(19) United States
(12) Patent Application Publication
(10) Pub. No.: US 2020/0164005 A1
(43) Pub. Date: May 28, 2020

(54) PHARMACEUTICAL COMPOSITIONS OF A BILE ACID DERIVATIVE AND MICROBIOME AND USES THEREOF

(71) Applicant: Intercept Pharmaceuticals, Inc., New York, NY (US)

(72) Inventors: Gary D Wu, Ardmore, PA (US); Hongzhe Lee, Penn Valley, PA (US); Farah Babakhani, San Diego, CA (US); Luciano Adorini, Milano (IT)

(21) Appl. No.: 16/486,873

(22) PCT Filed: Feb. 23, 2018
(86) PCT No.: PCT/US18/19451

§ 371 Date: Aug. 19, 2019

Related U.S. Application Data
(60) Provisional application No. 62/462,658, filed on Feb. 23, 2017.

(51) Int. Cl. A61K 35/747; A61K 9/00; A61K 31/575; A61K 35/745; A61K 35/742; A61K 35/744; A61K 35/741; A61P 1/00

(52) U.S. Cl. CPC A61K 35/747; A61K 9/0053; A61K 31/575; A61P 1/00; A61K 35/742; A61K 35/744; A61K 35/741; A61K 35/745

(57) ABSTRACT

The application relates to pharmaceutical compositions comprising a compound of formula I and one or more gut microbiome species, and methods of preparing and using the same.

### BACKGROUND

Mammalian hosts and gut microbiota have co-evolved where the former provide a uniquely suited environment in return for physiological benefits generated by the latter. Examples of the latter include the fermentation of indigestible carbohydrates to produce short chain fatty acids that are utilized by the host, biotransformation of conjugated bile acids, synthesis of certain vitamins, degradation of dietary oxalates, and education of the mucosal immune system. The metabolic properties of the gut microbiome are important in the response to a variety of drugs. Recent reports demonstrate the utility of using the characterization of the human gut microbiome as a modality to predict metabolic outcomes such as glucose homeostasis. Evaluating the gut microbiome and its metabolome may help predict clinically relevant outcomes.

The composition of the small intestine microbiota is subject to daily fluctuations, which are likely driven by response to dietary variation. Multiple reports using different sampling methods show predominance of Streptococcus spp. (accounting for 19% of 454-pyrosequencing reads). Other predominant genera include Veillonella spp. (13%), Prevotella spp. (12%), Rothia spp. (6.4%), Haemophilus spp. (5.7%), Actinobacillus spp. (5.5%), Escherichia spp. (4.6%), and Fusobacterium spp. (4.3%). At the phylum level, the distribution is: Firmicutes (43%), Proteobacteria (23%), Bacteroidetes (15%), Actinobacteria (9.3%), and Fusobacteria (7%). Culture-based methods have identified particular species of Streptococcus (S. salivarius, S. thermophilus, & S. parasanguinis) and Veillonella (V. dispar, V. parvula, V. rogosae, & V. atypica) in ileostomy effluent. Pyrosequencing revealed that abundance of Streptococcus (relative contribution ranging from 0.4-88.3%) and Veillonella spp (relative contribution ranging from <0.1-10.1%) was highly dependent on the time of day. The diet induced variability of the small intestinal gut microbiota, together with its potential to influence the pathogenesis of disease in human in both a therapeutic and preventative fashion, makes the alteration of either the composition or microbial biomass of the small intestine of particular interest in the field. Diet and bile acids interact in particularly important ways in the small intestine relevant to mammalian physiology. Bile acids play a critical role in small intestinal nutrient absorption and, in turn, nutrients in diet can lead to significant alterations in the delivery of bile acids into the small intestine. Furthermore, the gut microbiota has the unique ability to biochemically alter the structure of bile acids. In turn, bile acids can have a significant effect on the biology of bacteria where they have been shown to help shape the composition of the gut microbiota. Thus, there is a need for novel compositions comprising a bile acid or a derivative thereof and one or more gut microbes as a therapeutic agent, and methods of using a bile acid or a derivative thereof in combination with one or more gut microbes for treating or preventing diseases or disorders. The present application addresses the need.

### SUMMARY

The present application relates to a pharmaceutical composition comprising a compound of formula I:

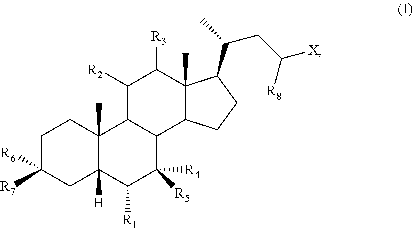

or a pharmaceutically acceptable salt or amino acid conjugate thereof, wherein R₁, R₂, R₃, R₄, R₅, R₆, R₇, R₈, X, m, and n are each as defined herein, and one or more gut microbiome species, and a pharmaceutically acceptable carrier.

The present application also relates to a method of treating or preventing an FXR mediated disease or condition or a disease or condition in which an abnormal composition of the gut microbiome is involved, comprising administering to a subject in need thereof a compound of the present application, or a pharmaceutically acceptable amino acid conjugate or salt thereof, and one or more gut microbiome species. In one embodiment, the present application relates to a method of treating. In one embodiment, the present application relates to a method of preventing.

The present application also relates to a compound of the present application, or a pharmaceutically acceptable amino acid conjugate or salt thereof, for use in combination with one or more gut microbiome species in treating or preventing an FXR mediated disease or condition or a disease or condition in which an abnormal composition of the gut microbiome is involved. In one embodiment, the present application relates to treating. In one embodiment, the present application relates to preventing.

The present application also relates to use of a compound of the present application, or a pharmaceutically acceptable amino acid conjugate or salt thereof, in the manufacture of a medicament for a combinational therapy with one or more gut microbiome species for the treatment or prevention of an FXR mediated disease or condition or a disease or condition in which an abnormal composition of the gut microbiome is involved. In one embodiment, the present application relates to treatment. In one embodiment, the present application relates to prevention.

The present application also relates to use of a compound of the present application, or a pharmaceutically acceptable amino acid conjugate or salt thereof, in combination with one or more gut microbiome species, in treating or preventing an FXR mediated disease or condition or a disease or condition in which an abnormal composition of the gut microbiome is involved. In one embodiment, the present application relates to treating. In one embodiment, the present application relates to preventing.

The present application also relates to a method of enhancing the efficacy of an FXR ligand in treating or preventing a disease or condition, comprising administering to a subject in need thereof one or more gut microbiome species. In one embodiment, the present application relates to a method of treating. In one embodiment, the present application relates to a method of preventing.

The present application also relates to one or more gut microbiome species, for use in enhancing the efficacy of an FXR ligand in treating or preventing a disease or condition. In one embodiment, the present application relates to treating. In one embodiment, the present application relates to preventing.

The present application also relates to use of one or more gut microbiome species in the manufacture of a medicament for enhancing the efficacy of an FXR ligand in the treatment or prevention of a disease or condition. In one embodiment, the present application relates to treatment. In one embodiment, the present application relates to prevention.

The present application also relates to use of one or more gut microbiome species in enhancing the efficacy of an FXR ligand in treating or preventing a disease or condition. In one embodiment, the present application relates to treating. In one embodiment, the present application relates to preventing.

The details of the application are set forth in the accompanying description below. Although methods and materials similar or equivalent to those described herein can be used in the practice or testing of the present application, illustrative methods and materials are now described. In the case of conflict, the present specification, including definitions, will control. In addition, the materials, methods, and examples are illustrative only and are not intended to be limiting. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs.

The contents of all references (including literature references, issued patents, published patent applications, and co-pending patent applications) cited throughout this application are hereby expressly incorporated herein in their entireties by reference. The references cited herein are not admitted to be prior art to the application.

### BRIEF DESCRIPTION OF THE DRAWINGS

FIG. 1: Box plots showing the relative abundance of gram-positive Lactobacillus casei paracasei (left plot) and gram-positive Streptococcus thermophilus (right plot) over time in fecal samples collected from humans related with the indicated dose of OCA (5 mg, 10 mg, or 25 mg).

FIG. 2: Graphs showing the relative abundance of gram-positive Streptococcus thermophilus (left graphs) over time in samples collected from humans treated with 5 mg OCA, and the levels of plasma C4 (7α-hydroxy-4-cholesten-3-one, a bile acid precursor) over time in samples collected from the same humans (right graphs).

FIG. 3: Graphs showing the relative abundance of gram-positive Lactobacillus casei paracasei (left graphs) over time in samples collected from humans treated with 5 mg OCA, and the levels of plasma C4 over time in samples collected from the same humans (right graphs).

FIG. 4: Graphs showing the relative abundance of gram-negative Alistipes shahii (left graphs) over time in samples collected from humans treated with 5 mg OCA, and the levels of C4 over time in samples collected from the same subjects (right graphs).

FIG. 5: Graphs showing the relative abundance of gram-negative Odoribacter splanchnicus (left graphs) over time in samples collected from humans treated with 5 mg OCA, and the levels of C4 over time in samples collected from the same subjects (right graphs).

FIG. 6: A multidimensional scaling (MDS) plot showing the most differentially abundant genes over time (repeated measure ANOVA, FDR (false discovery rate)<0.01 for time effect).

FIG. 7A: A heat map showing the most differentially abundant genes over time (repeated measure ANOVA, FDR<0.01 for time effect). Distance was calculated by 1-kendall correlation.

FIG. 7B: A table showing the result of a UniRef search of transposases and their association with specific bacterial taxa.

FIG. 8: A MDS plot showing the most differentially abundant MetaCyc pathways over time (repeated measure ANOVA, FDR<0.01 for time effect).

FIG. 9: A heat map showing the most differentially abundant MetaCyc pathways over time (repeated measure ANOVA, FDR<0.01 for time effect). Distance was calculated by 1-kendall correlation.

FIG. 10: A MDS plot showing the most differentially abundant KEGG pathways over time (repeated measure ANOVA, FDR<0.01 for time effect).

FIG. 11: A heat map showing the most differentially abundant KEGG pathways over time (repeated measure ANOVA, FDR<0.01 for time effect). Distance was calculated by 1-kendall correlation.

FIG. 12: Box plots showing the abundance of FGF19 (Fibroblast growth factor 19) and the top two genes associated with FGF19 over time at OCA dose of 5 mg or 10 mg.

FIG. 13: Graphs showing the relative abundance of gram-positive Streptococcus thermophilus (left two graphs) over time in samples collected from humans treated with 10 mg OCA, and the levels of C4 over time in samples collected from the same subjects (right two graphs).

FIG. 14: Graphs showing the relative abundance of gram-positive Lactobacillus casei paracasei (left two graphs) over time in samples collected from humans treated with 10 mg OCA, and the levels of C4 over time in samples collected from the same subjects (right two graphs).

FIG. 15: Graphs showing the relative abundance of Alistipes putredinis (left two graphs) over time in samples collected from humans treated with 10 mg OCA, and the levels of C4 over time in samples collected from the same subjects (right two graphs).

FIG. 16: A graph showing the change in FGF19 level in samples from humans treated with 5 mg or 10 mg OCA.

FIG. 17: Graphs showing the change in FGF19 level in samples from humans treated with 5 mg (top graph) or 10 mg (bottom graph) OCA.

FIG. 18: Box plots showing the relative abundance of Bacteroides uniformis (left plot) and Streptococcus thermophilus (right plot) over time in samples collected from humans treated with the indicated dose of OCA (5 mg, 10 mg, or 25 mg).

FIG. 19: Graphs showing the relative abundance of gram-positive Ruminococcus torques (left graphs) over time in samples collected from humans treated with 5 mg OCA, and the levels of FGF19 over time in samples collected from the same subjects (right graphs).

FIG. 20: Graphs showing the relative abundance of gram-positive Coprobacillus unclassified (left graphs) over time in samples collected from humans treated with 5 mg OCA, and the levels of FGF19 over time in samples collected from the same subjects (right graphs).

FIG. 21: Graphs showing the relative abundance of gram-positive Clostridium symbiosum (left graphs) over time in samples collected from humans treated with 5 mg OCA, and the levels of FGF19 over time in samples collected from the same subjects (right graphs).

FIG. 22: Graphs showing the relative abundance of gram-positive Lactococcus lactis (left graphs) over time in samples collected from humans treated with 5 mg OCA, and the levels of FGF19 over time in samples collected from the same subjects (right graphs).

FIG. 23: Graphs showing the relative abundance of gram-negative E. coli (left graphs) over time in samples collected from humans treated with 5 mg OCA, and the levels of FGF19 over time in samples collected from the same subjects (right graphs).

FIG. 24: Graphs showing the relative abundance of gram-negative Akkermansia muciniphila (left graphs) over time in samples collected from humans treated with 5 mg OCA, and the levels of FGF19 over time in samples collected from the same subjects (right graphs).

FIG. 25: Graphs showing the relative abundance of gram-positive Ruminococcus bromii (left graphs) over time in samples collected from humans treated with 5 mg OCA, and the levels of FGF19 over time in samples collected from the same subjects (right graphs).

FIG. 26: Graphs showing the relative abundance of gram-positive Streptococcus thermophilus (left graphs) over time in samples collected from humans treated with 10 mg OCA, and the levels of FGF19 over time in samples collected from the same subjects (right graphs).

FIG. 27: Graphs showing the relative abundance of gram-positive Lactococcus lactis (left graphs) over time in samples collected from humans treated with 10 mg OCA, and the levels of FGF19 over time in samples collected from the same subjects (right graphs).

FIG. 28: Graphs showing the relative abundance of gram-negative Bacteroides ovatus (left graphs) over time in samples collected from humans treated with 10 mg OCA, and the levels of FGF19 over time in samples collected from the same subjects (right graphs).

FIG. 29: Graphs showing the relative abundance of gram-positive Lactobacillus casei paracasei (left graphs) over time in samples collected from humans treated with 10 mg OCA, and the levels of FGF 19 over time in samples collected from the same subjects (right graphs).

FIG. 30: Graphs showing the relative abundance of gram-negative Veillonella unclassified (left graphs) over time in samples collected from humans treated with 10 mg OCA, and the levels of FGF19 over time in samples collected from the same subjects (right graphs).

FIG. 31: Graphs showing the relative abundance of Lachnospiracea bacterium 5_1_63FAA (left two graphs) over time in samples collected from humans treated with 10 mg OCA, and the levels of C4 over time in samples collected from the same subjects (right two graphs).

FIG. 32: Graphs showing the relative abundance of Bifidobacterium breve (left two graphs) over time in samples collected from humans treated with 10 mg OCA, and the levels of C4 over time in samples collected from the same subjects (right two graphs).

FIG. 33: Graphs showing the relative abundance of Lactococcus lactis (left two graphs) over time in samples collected from humans treated with 10 mg OCA, and the levels of C4 over time in samples collected from the same subjects (right two graphs).

FIG. 34: Graphs showing the relative abundance of Streptococcus salivarius (left two graphs) over time in samples collected from humans treated with 10 mg OCA, and the levels of C4 over time in samples collected from the same subjects (right two graphs).

FIG. 35: Graphs showing the relative abundance of Subdoligranulum unclassified (left two graphs) over time in samples collected from humans treated with 10 mg OCA, and the levels of C4 over time in samples collected from the same subjects (right two graphs).

FIG. 36: Graphs showing the relative abundance of Lachnospiraceae bacterium 3_1_57FAA_CT1 (left two graphs) over time in samples collected from humans treated with 10 mg OCA, and the levels of C4 over time in samples collected from the same subjects (right two graphs).

FIG. 37: Graphs showing the relative abundance of Dorea longicatena (left two graphs) over time in samples collected from humans treated with 10 mg OCA, and the levels of C4 over time in samples collected from the same subjects (right two graphs).

FIG. 38: Graphs showing the relative abundance of Bacteroidales bacterium ph8 (left two graphs) over time in samples collected from humans treated with 10 mg OCA, and the levels of C4 over time in samples collected from the same subjects (right two graphs).

FIG. 39: Graphs showing the relative abundance of Bifidobacterium longum (left two graphs) over time in samples collected from humans treated with 10 mg OCA, and the levels of C4 over time in samples collected from the same subjects (right two graphs).

FIG. 40: Graphs showing the relative abundance of Bacteroides plebeius (left two graphs) over time in samples collected from humans treated with 10 mg OCA, and the levels of C4 over time in samples collected from the same subjects (right two graphs).

FIG. 41: Graphs showing the relative abundance of Ruminococcus obeum (left two graphs) over time in samples collected from humans treated with 10 mg OCA, and the levels of C4 over time in samples collected from the same subjects (right two graphs).

FIG. 42: Graphs showing the relative abundance of Paraprevotella clara (left two graphs) over time in samples collected from humans treated with 10 mg OCA, and the levels of C4 over time in samples collected from the same subjects (right two graphs).

FIG. 43: Graphs showing the relative abundance of Clostridium spiroforme (left two graphs) over time in samples collected from humans treated with 10 mg OCA, and the levels of C4 over time in samples collected from the same subjects (right two graphs).

FIG. 44: Graphs showing the relative abundance of Paraprevotella unclassified (left two graphs) over time in samples collected from humans treated with 10 mg OCA, and the levels of C4 over time in samples collected from the same subjects (right two graphs).

FIG. 45: Graphs showing the relative abundance of Bacteroide uniformis (left two graphs) over time in samples collected from humans treated with 5 mg OCA, and the levels of C4 over time in samples collected from the same subjects (right two graphs).

FIG. 46: Graphs showing the relative abundance of E. coli (left two graphs) over time in samples collected from humans treated with 5 mg OCA, and the levels of C4 over time in samples collected from the same subjects (right two graphs).

FIG. 47: Graphs showing the relative abundance of Streptococcus parasanguinis (left two graphs) over time in samples collected from humans treated with 5 mg OCA, and the levels of C4 over time in samples collected from the same subjects (right two graphs).

FIG. 48: Graphs showing the relative abundance of Ruminococcus gnavus (left two graphs) over time in samples collected from humans treated with 5 mg OCA, and the levels of C4 over time in samples collected from the same subjects (right two graphs).

FIG. 49: Graphs showing the relative abundance of Eubacterium ramulus (left two graphs) over time in samples collected from humans treated with 5 mg OCA, and the levels of C4 over time in samples collected from the same subjects (right two graphs).

FIG. 50: Graphs showing the relative abundance of Anaerotruncus unclassified (left two graphs) over time in samples collected from humans treated with 5 mg OCA, and the levels of C4 over time in samples collected from the same subjects (right two graphs).

FIG. 51: Graphs showing the relative abundance of Lachnospiraceae bacterium 8_1_57FAA (left two graphs) over time in samples collected from humans treated with 5 mg OCA, and the levels of C4 over time in samples collected from the same subjects (right two graphs).

FIG. 52: Graphs showing the relative abundance of Coprococcus sp ART55-1 (left two graphs) over time in samples collected from humans treated with 5 mg OCA, and the levels of C4 over time in samples collected from the same subjects (right two graphs).

FIGS. 53A-53D: Heat maps showing the percentage reduction in growth of the indicated strains, as compared to controls (no bile acid treatment), treated with different concentrations of GCDCA (FIG. 53A), GCA (FIG. 53B), TCA (FIG. 53C), and OCA (FIG. 53D). Strains in dashed boxes are gram-positive, and strains outside the dashed boxes are gram-negative. Estimated physiologically relevant small intestinal luminal concentrations of endogenous bile acids are marked with “*”, estimated physiologically relevant small intestinal luminal concentration of OCA in mice (10 mg/kg/day) is marked with “#”, and estimated physiologically relevant small intestinal luminal concentration in humans (10 mg/day) is marked with “$”.

FIG. 54: Bar graphs showing the concentration of taurocholic acid and taurodeoxycholic acid at proximal small bowel and distal small bowel, or in feces, in samples collected from mice treated with control (methylcellulose) or OCA (10 mg/kg/day) for 14 days, followed by no treatment for additional 14 days. Statistically significant differences based on two-tailed Student t-tests are noted: p<0.05 (*) and p<0.01 (**).

FIGS. 55A-55D: Linear and box and whisker plots of: (FIG. 55A, FIG. 55C) plasma C4 levels and (FIG. 55B, FIG. 55D) S. thermophilus relative abundance in the 10 mg OCA group.

FIGS. 56A-56E: Genomic signature of the fecal microbiome associated with OCA administration. FIG. 56A shows a multidimensional scaling (MDS) plot of samples based on the Kendall rank correlation coefficient derived from 782 genes with a time-dependent effect in response to OCA administration based on day of the study (repeated measure ANOVA, FDR<0.01). FIG. 56B shows distribution of the 782 genes by bacterial taxonomy. FIG. 56C shows the abundance of a selected transposase (V8LYU6, from S. thermophilus) over time. FIG. 56D shows mean abundance of 32 transposases out of 394 total transposases identified in the samples, having significant time-dependent responses to each of the three OCA doses. FIG. 56E shows ROC curves for transposases and plasma C4.

FIGS. 57A-57C: Bacterial metabolic pathways associated with OCA administration. FIG. 57A shows the 135 metabolic pathways that were significantly associated with OCA administration (repeated measure ANOVA, FDR<0.01) categorized by bacterial taxa. FIG. 57B shows a MDS plot of samples based on the Kendall rank correlation coefficient derived from the 135 metabolic pathways that show a significant association with OCA administration. FIG. 57C shows a heatmap of significantly altered metabolic pathways from three major bacterial species sorted by time and dose.

FIGS. 58A-58B: Minimal inhibitory concentrations (MICs) of selected Gram-positive bacterial species in response treatment with two endogenous bile acids and OCA. FIG. 58A shows MICs of selected Gram-positive bacteria species, most strongly associated with the use of OCA, in response to treatment with the two dominant conjugated primary bile acids found in the human small intestine, glycochenodeoxycholic acid (GCDCA) and glycocholic acid (GCA), under both aerobic and anaerobic conditions. N=3 per measurement. FIG. 58B shows MICs of the same bacterial taxa in response to treatment with OCA. N=3 per measurement.

FIGS. 59A-59E: Effect of OCA administration on luminal bile acid concentrations in the murine small intestine and feces. FIG. 59A-59C shows total (endogenous bile acids and OCA), total endogenous, total primary, and total secondary bile acids in the lumen of the proximal small intestine (FIG. 59A); in the lumen of the distal small intestine (FIG. 59B); and in the feces of mice (FIG. 59C) following 14 days of gavage with either water (control, N=5), 0.5% methylcellulose (MC, N=10), or 0.5% methylcellulose with 10 mg/kg obeticholic acid (OCA, N=10). Mean+SE, *p<0.05, **p<0.01, ***p<0.001. FIGS. 59D-59E show heatmaps of luminal bile acid concentrations in the proximal (FIG. 59D) and distal (FIG. 59E) small intestine.

FIG. 60: The effect of OCA on the composition of the proximal and distal small intestinal, as well as the feces, microbiota of mice based on 16S tagged sequencing.

FIG. 61A-61C: The discriminatory power of the relative abundance of bacterial species to discriminate OCA treatment (day 16) vs. non-treatment (days 1 and 37), where the discriminatory power of each species was assessed by logistic regression models. FIG. 61A shows the three species with the highest AUC values based on a ROC analysis of the three OCA doses. FIG. 61B shows AUC values based on a ROC analysis using the combination of any two of the three species with the highest AUC values. FIG. 61C shows AUC values based on separate ROC analyses for Day 1 vs. Day 16 and Day 37 vs. Day 16 based on logistic regression analysis.

FIG. 62A-62B: Design of an open label, randomized, single dose and multiple dose trial to assess the pharmacokinetics of obeticholic acid (OCA). FIG. 62A shows design of the study, where three groups received 5, 10, or 25 mg/day of OCA (eight healthy human subjects, four male and four female, randomized into each group). FIG. 62B shows plasma C4 levels over time in the 10 mg OCA group.

FIG. 63: Box and whisker plots of differentially abundant taxa in the distal small intestine of mice in response to treatment with OCA relative to two controls. S24-7, Clostridiaceae, and Turibacter are differentially abundant between the MC and OCA groups (fdr=0.1759, 0.04503, and 0.2332, respectively); Sutterella and Akkermansia are differentially abundant between the control and OCA groups (fdr=0.3199 for both).

FIGS. 64A-64F: The power of plasma C4 levels to predict OCA treatment. FIGS. 64A-64F shows result of ROC analysis of plasma C4 levels on two OCA dose groups together (FIG. 64A and FIG. 64B), on 5 mg OCA group (FIG. 64C and FIG. 64D), and on 10 mg OCA group (FIG. 64E and FIG. 64F).

### DETAILED DESCRIPTION

The human gut microbiome (microbes, their genomes, and their environment) and the microbiota (microrganisms alone) describe the microbial populations that live in the intestine of humans. The gut microbiota contains tens of trillions of microorganisms (e.g., bacteria, virus, fungi, and archaea), including at least 1000 different species of known bacteria with more than 3 million genes. The gut microbiome performs important physiological functions, including: biodegradation of glycans to help the body digest plant and animal derived dietary glycans, production of short chain fatty acids, which serve as nutrients for healthy gut epithelial cells, production of vitamins (B and K) and essential amino acids, colonization resistance that inhibits colonization and overgrowth of invading pathogenic microbes, and regulation of the immune system.

The composition of the gut microbiota is established early on in life and is affected by many factors including perinatal mode of delivery, feeding mode, diet, genetics, intestinal mucin glycosylation that affects bacterial colonization, and the environment. Once established, the microbiota, at the phylum level, remains fairly stable throughout the adult life and changes with diet, infections, antibiotics and other medications, surgery or other life style changes. The two dominant bacterial phyla recognized in adult life are Frimicutes and Bacteroidetes, however, the relative proprotions of them varies in individuals. The diversity within each individuals is at the level of bacterial species and is influenced by environmental factors and host genetics. Additionally, distinct microenvironments exist within the the intestine. The microbiota detected in stool samples, which are representative of luminal microbiota, is distint from the microbial communinites that are associated with the mucosal surfaces. Shifts from a healthy microbiota (dysbiosis) can be associated with disease state. Additionally, as adults age and become sick or during their residency in institutions, their microbiome can shif and may become less diverse.

Many studies have demonstrated the beneficial effects of prebiotics and probiotics on our gut microbiota. Serving as “food” for beneficial bacteria, prebiotics help improve the functioning of microbiota while allowing the growth and activity of some “good” bacteria. Present in some fermented products such as yoghourt, probiotics help gut microbiota keep its balance, integrity and diversity. Probiotics are live micro-organisms that, when administered, confer a health benefit to the host. Most are facultative anaerobes belonging to a number of genera such as Streptoccoci, Lactobacilli, Esherichia, and Bifidobacteria. Most have marginal health benefits possibly because they are not able to establish a robust niche within the intestinal tract based on analysis of fecal samples. However, it is possible that they may exist at higher proportional abundance in the small intestine since many of these same genera have been described to be the predominant bacterial taxa within the small intestine of mice and humans. Although proportional abundance may be high for these organisms, absolute abundance is very low and likely to be at least 6 logs lower in the small intestine than in the colon. Unfortunately, very limited information about the composition, biomass, and dynamics of the human small intestinal microbiota has been characterized. There is growing evidence that the small bowel microbiota may be quite important for the pathogenesis of disease that involves a disruption of barrier function, amongst others.

There is a bidirectional interaction between the gut microbiota and bile acids: bile acids can have bacteriostatic effects and the gut microbiota can modify primary bile acids into secondary bile acids. It has been shown that bile acids have both direct antimicrobial effects on gut microbes, and indirect effects through FXR-induced antimicrobial peptides. For example, the potency of deoxycholic acid (DCA) as an antimicrobial agent, is an order of magnitude greater than cholic acid (CA), owing to its hydrophobicity and detergent properties on bacterial membranes. Indeed, complex and significant changes in the gut microbiome are observed when rats are fed bile acids.

Obeticholic acid (OCA) is a modified bile acid and farnesoid X receptor (FXR) agonist that is 100-fold more potent than the endogenous FXR agonist CDCA, making it an attractive novel therapeutic agent for FXR mediated disease or condition, such as cholestatic liver disease, NAFLD, and NASH, due to its FXR-mediated effects including the suppression of bile acid synthesis.

The suppression of bile acid synthesis can be also quantified by the reduction in plasma levels of 7α-hydroxy-4-cholesten-3-one (C4). Fibroblast growth factor 19 (FGF 19), synthesized in the ileum in response to bile acid absorption, enters the portal venous circulation and inhibits new bile acid synthesis in the liver, thus providing negative feedback.

The present application relates to a pharmaceutical composition comprising a compound of formula I:

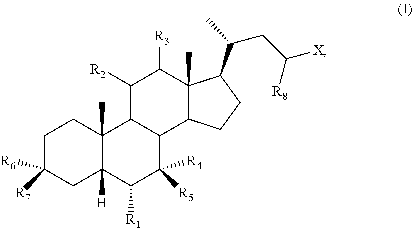

or a pharmaceutically acceptable salt or amino acid conjugate thereof, wherein:
R₁ is unsubstituted C₁-C₆ alkyl;
R₂ is H or hydroxyl;
R₃ is H or hydroxyl;
R₄, R₅, R₆, and R₇ are each independently H or hydroxyl;
R₈ is H or unsubstituted C₁-C₆ alkyl;
X is C(O)OH, C(O)NH(CH₂)ₘSO₃H, C(O)NH(CH₂)ₙCO₂H, or OSO₃H;
m is 1, 2, or 3; and
n is 1, 2, or 3,
   and one or more gut microbiome species, and a pharmaceutically acceptable carrier.

In one embodiment, R₁ is methyl, ethyl, propyl (e.g., n-propyl or i-propyl), butyl (e.g., i-butyl, s-butyl, or t-butyl), pentyl, or hexyl. In one embodiment, R₁ is methyl, ethyl, or propyl (e.g., n-propyl or i-propyl). In one embodiment, R₁ is methyl or ethyl. In one embodiment, R₁ is ethyl.

In one embodiment, R₂ is H. In one embodiment, R₂ is hydroxyl.

In one embodiment, R₃ is H. In one embodiment, R₃ is hydroxyl.

In one embodiment, R₄ is H and R₅ is hydroxyl. In one embodiment, R₄ is hydroxyl and R₅ is H. In one embodiment, R₄ and R₅ are each H.

In one embodiment, R₆ is H and R₇ is hydroxyl. In one embodiment, R₆ is hydroxyl and R₇ is H. In one embodiment, R₆ and R₇ are each H.

In one embodiment, R₈ is H. In one embodiment, R₈ is methyl, ethyl, propyl (e.g., n-propyl or i-propyl), butyl (e.g., i-butyl, s-butyl, or t-butyl), pentyl, or hexyl. In one embodiment, R₈ is methyl, ethyl, or propyl (e.g., n-propyl or i-propyl). In one embodiment, R₈ is methyl or ethyl. In one embodiment, R₈ is methyl.

In one embodiment, X is C(O)OH, C(O)NH(CH₂)ₘSO₃H, or C(O)NH(CH₂)ₙCO₂H.

In one embodiment, X is C(O)OH, C(O)NH(CH₂)SO₃H, C(O)NH(CH₂)CO₂H, C(O)NH(CH₂)₂SO₃H, or C(O)NH(CH₂)₂CO₂H. In one embodiment, X is C(O)OH. In one embodiment, X is OSO₃H.

In one embodiment, m is 1. In one embodiment, m is 2. In one embodiment, m is 3.

In one embodiment, n is 1. In one embodiment, n is 2. In one embodiment, n is 3.

In one embodiment, a compound of formula I is of formula Ia:

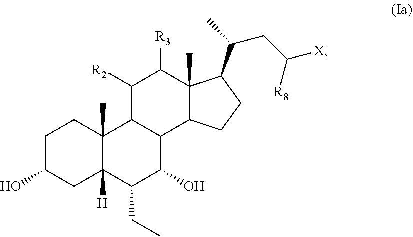

or a pharmaceutically acceptable salt or amino acid conjugate thereof, wherein R₂, R₃, R₈, X, m, and n are each as defined above in formula I.

In one embodiment, a compound of formula I is of formula Ib-1 or Ib-2:

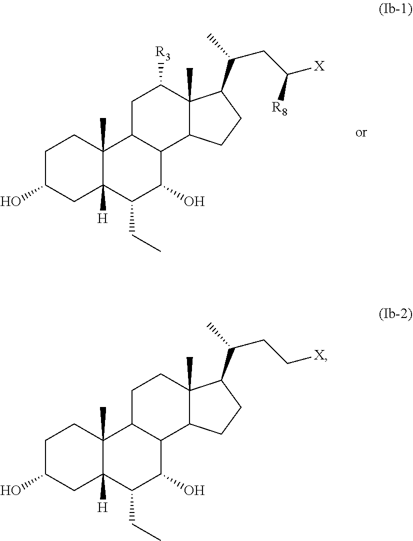

or a pharmaceutically acceptable salt or amino acid conjugate thereof, wherein R₃, R₈, X, m, and n are each as defined above in formula I.

In one embodiment, a compound of formula I is of formula Ic:

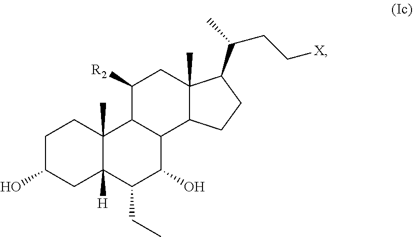

or a pharmaceutically acceptable salt or amino acid conjugate thereof, wherein R₂, X, m, and n are each as defined above in formula I.

In any one of formulae described herein, any of the substituents described above for any of R₁, R₂, R₃, R₄, R₅, R₆, R₇, R₈, X, m, and n can be combined with any of the substituents described above for the remainder of R₁, R₂, R₃, R₄, R₅, R₆, R₇, R₈, X, m, and n.

In one embodiment, R₂ is H and R₃ is H. In one embodiment, R₂ is H, R₃ is H, and R₁ is unsubstituted C₁-C₆ alkyl. In one embodiment, R₂ is H, R₃ is H, R₁ is unsubstituted C₁-C₆ alkyl, and R₈ is H. In one embodiment, R₂ is H, R₃ is H, and R₁ is methyl or ethyl. In one embodiment, R₂ is H, R₃ is H, R₁ is methyl or ethyl, and R₈ is H. In one embodiment, R₂ is H, R₃ is H, R₁ is methyl or ethyl, R₈ is H, and X is C(O)OH, C(O)NH(CH₂)ₘSO₃H, or C(O)NH(CH₂)ₙCO₂H. In one embodiment, R₂ is H, R₃ is H, R₁ is methyl or ethyl, R₈ is H, and X is C(O)OH. In one embodiment, R₂ is H, R₃ is H, R₁ is methyl or ethyl, R₈ is H, and X is OSO₃H. In one embodiment, a compound of formula I is of formula Ib-2, and X is as defined herein in this paragraph.

In one embodiment, R₂ is H and R₃ is hydroxyl. In one embodiment, R₂ is H, R₃ is hydroxyl, and R₁ is unsubstituted C₁-C₆ alkyl. In one embodiment, R₂ is H, R₃ is hydroxyl, R₁ is unsubstituted C₁-C₆ alkyl, and R₈ is unsubstituted C₁-C₆ alkyl. In one embodiment, R₂ is H, R₃ is hydroxyl, and R₁ is methyl or ethyl. In one embodiment, R₂ is H, R₃ is hydroxyl, R₁ is methyl or ethyl, and R₈ is unsubstituted C₁-C₆ alkyl. In one embodiment, R₂ is H, R₃ is hydroxyl, R₁ is unsubstituted C₁-C₆ alkyl, and R₈ is methyl. In one embodiment, R₂ is H, R₃ is hydroxyl, R₁ is methyl or ethyl, and R₈ is methyl. In one embodiment, R₂ is H, R₃ is hydroxyl, R₁ is methyl or ethyl, R₈ is methyl, and X is C(O)OH, C(O)NH(CH₂)ₘSO₃H, or C(O)NH(CH₂)ₙCO₂H. In one embodiment, R₂ is H, R₃ is hydroxyl, R₁ is methyl or ethyl, R₈ is methyl, and X is C(O)OH. In one embodiment, a compound of formula I is of formula Ib-1, and R₃, R₈, and X are as defined herein in this paragraph.

In one embodiment, R₂ is hydroxyl and R₃ is H. In one embodiment, R₂ is hydroxyl, R₃ is H, and R₁ is unsubstituted C₁-C₆ alkyl. In one embodiment, R₂ is hydroxyl, R₃ is H, R₁ is unsubstituted C₁-C₆ alkyl, and R₈ is H. In one embodiment, R₂ is hydroxyl, R₃ is H, and R₁ is methyl or ethyl. In one embodiment, R₂ is hydroxyl, R₃ is H, R₁ is methyl or ethyl, and R₈ is H. In one embodiment, R₂ is hydroxyl, R₃ is H, R₁ is methyl or ethyl, R₈ is H, and X is C(O)OH, C(O)NH(CH₂)ₘSO₃H, or C(O)NH(CH₂)ₙCO₂H. In one embodiment, R₂ is hydroxyl, R₃ is H, R₁ is methyl or ethyl, R₈ is H, and X is C(O)OH. In one embodiment, a compound of formula I is of formula Ic, and R₂ and X are as defined herein in this paragraph.

In one embodiment, R₂, R₃, R₈, and X are defined and combined, where applicable, in the preceding paragraphs, and R₁ is ethyl.

In one embodiment, R₁, R₂, R₃, R₈, and X are defined and combined, where applicable, in the preceding paragraphs, and R₄ is hydroxyl, R₅ is H, R₆ is hydroxyl, and R₇ is H.

In one embodiment, the compound of the present application is:

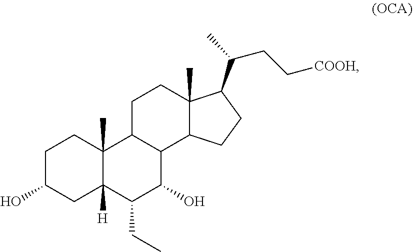 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or a pharmaceutically acceptable salt or amino acid conjugate thereof.

In one embodiment, the compound of the present application is:

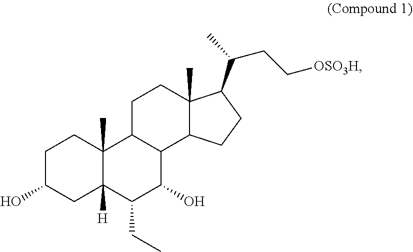

or a pharmaceutically acceptable salt or amino acid conjugate thereof.

In one embodiment, the compound of the present application is:

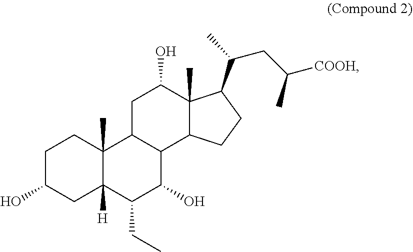 [SMILES: CC[C@H]1[C@@H](O)C2C3CC[C@H]([C@H](C)C[C@H](C)C(=O)O)[C@@]3(C)[C@@H](O)CC2[C@@]2(C)CC[C@@H](O)C[C@@H]12]

or a pharmaceutically acceptable salt or amino acid conjugate thereof.

In one embodiment, the compound of the present application is:

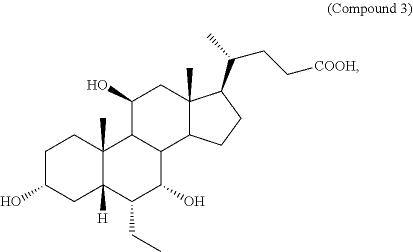 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

or a pharmaceutically acceptable salt or amino acid conjugate thereof.

In one embodiment, the compound of the present application is a pharmaceutically acceptable salt. In one embodiment, the pharmaceutically acceptable salt is a sodium salt (e.g., OSO₃⁻Na⁺). In one embodiment, the pharmaceutically acceptable salt is triethylamine salt (e.g., X is OSO₃⁻NHEt₃⁺).

In one embodiment, the one or more gut microbiome species is a member in a family selected from: Actinomycetaceae, Bogoriellaceae, Brevibacteriaceae, Cellulomonadaceae, Acholeplasmataceae, Acidithiobacillaceae, Alcanivoracaceae, Alteromonadaceae, Blattabacteriaceae, Cardiobacteriaceae, Chlamydiaceae, Chromatiaceae, Clostridiales Family XIII. Incertae Sedis, Cyclobacteriaceae, Dehalococcoidaceae, Desulfobacteraceae, Desulfobulbaceae, Ectothiorhodospiraceae, Elusimicrobiaceae, Entomoplasmataceae, Erythrobacteraceae, Gallionellaceae, Halanaerobiaceae, Jonesiaceae, Kofleriaceae, Leptospiraceae, Methanobacteriaceae, Methylococcaceae, Methylophilaceae, Myxococcaceae, Nitrosomonadaceae, Nitrospiraceae, Oceanospirillaceae, Oscillospiraceae, Piscirickettsiaceae, Propionibacteriaceae, Pseudoalteromonadaceae, Puniceicoccaceae, Rickettsiaceae, Rubrobacteraceae, Shewanellaceae, Spirochaetaceae, Spiroplasmataceae, Sutterellaceae, Syntrophomonadaceae, Thermaceae, Corynebacteriaceae, Dermabacteraceae, Dietziaceae, Geodermatophilaceae, Gordoniaceae, Intrasporangiaceae, Microbacteriaceae, Micrococcaceae, Micromonosporaceae, Mycobacteriaceae, Nocardiaceae, Promicromonosporaceae, Propionibacterineae, Streptomycetaceae, Micrococcineae, Bifidobacteriaceae, Coriobacteriaceae, Deinococcaceae, Halobacteroidaceae, Alicyclobacillaceae, Bacillaceae, Bacillales Incertae Sedis XI, Listeriaceae, Paenibacillaceae, Planococcaceae, Staphylococcaceae, Aerococcaceae, Carnobacteriaceae, Enterococcaceae, Lactobacillaceae, Leuconostocaceae, Streptococcaceae, Christensenellaceae, Clostridiaceae, Ruminococcaceae, Family XIII Incertae Sedis, Peptostreptococcaceae, Family XI Incertae Sedis, Lachnospiraceae, Eubacteriaceae, Erysipelotrichaceae, Erysipelotrichaceae XVI, Erysipelotrichaceae XVII, Erysipelotrichaceae XVIII, Acidiaminococcaceae, Peptococcaceae, Veillonellaceae, Bacteroidaceae, Porphyromonadaceae, Prevotellaceae, Rikenellaceae, Cytophagaceae, Flavobacteriaceae, Chitinophagaceae, Sphingobacteriaceae, Fusobacteriaceae, Leptotrichiaceae, Victivallaceae, Planctomycetaceae, Caulobacteraceae, Aurantimonadaceae, Bradyrhizobiaceae, Brucellaceae, Hyphomicrobiaceae, Methylobacteriaceae, Phyllobacteriaceae, Rhizobiaceae, Xanthobacteraceae, Rhodobacteraceae, Acetobacteraceae, Rhodospirillaceae, Sphingomonadaceae, Alcaligenaceae, Burkholderiaceae, Comamonadaceae, Oxalobacteraceae, Suterellaceae, Neisseriaceae, Rhodocyclaceae, Desulfovibrionaceae, Campylobacteraceae, Helicobacteraceae, Aeromonadaceae, Succinivibrionaceae, Enterobacteriaceae, Pasteurellaceae, Moraxellaceae, Pseudomonadaceae, Vibrionaceae, Sinobacteraceae, Xanthomonadaceae, Brachyspiraceae, Synergistaceae, Mycoplasmataceae, and Verrucomicrobiaceae.

In one embodiment, the one or more gut microbiome species is gram positive. In one embodiment, the one or more gut microbiome species is a member in a family selected from: Actinomycetaceae, Bogoriellaceae, Brevibacteriaceae, Cellulomonadaceae, Corynebacteriaceae, Dermabacteraceae, Dietziaceae, Geodermatophilaceae, Gordoniaceae, Intrasporangiaceae, Microbacteriaceae, Micrococcaceae, Micromonosporaceae, Mycobacteriaceae, Nocardiaceae, Promicromonosporaceae, Propionibacterineae, Streptomycetaceae, Micrococcineae, Bifidobacteriaceae, Coriobacteriaceae, Deinococcaceae, Halobacteroidaceae, Alicyclobacillaceae, Bacillaceae, Bacillales Incertae Sedis XI, Listeriaceae, Paenibacillaceae, Planococcaceae, Staphylococcaceae, Aerococcaceae, Carnobacteriaceae, Enterococcaceae, Lactobacillaceae, Leuconostocaceae, Streptococcaceae, Christensenellaceae, Clostridiaceae, Ruminococcaceae, Family XIII Incertae Sedis, Peptostreptococcaceae, Family XI Incertae Sedis, Lachnospiraceae, Eubacteriaceae, Erysipelotrichaceae, Erysipelotrichaceae XVI, Erysipelotrichaceae XVII, Erysipelotrichaceae XVIII, Acidiaminococcaceae, Peptococcaceae, and Veillonellaceae.

In one embodiment, the one or more gut microbiome species is gram negative. In one embodiment, the one or more gut microbiome species is a member in a family selected from: Bacteroidaceae, Porphyromonadaceae, Prevotellaceae, Rikenellaceae, Cytophagaceae, Flavobacteriaceae, Chitinophagaceae, Sphingobacteriaceae, Fusobacteriaceae, Leptotrichiaceae, Victivallaceae, Planctomycetaceae, Caulobacteraceae, Aurantimonadaceae, Bradyrhizobiaceae, Brucellaceae, Hyphomicrobiaceae, Methylobacteriaceae, Phyllobacteriaceae, Rhizobiaceae, Xanthobacteraceae, Rhodobacteraceae, Acetobacteraceae, Rhodospirillaceae, Sphingomonadaceae, Alcaligenaceae, Burkholderiaceae, Comamonadaceae, Oxalobacteraceae, Suterellaceae, Neisseriaceae, Rhodocyclaceae, Desulfovibrionaceae, Campylobacteraceae, Helicobacteraceae, Aeromonadaceae, Succinivibrionaceae, Enterobacteriaceae, Pasteurellaceae, Moraxellaceae, Pseudomonadaceae, Vibrionaceae, Sinobacteraceae, Xanthomonadaceae, Brachyspiraceae, Synergistaceae, Mycoplasmataceae, and Verrucomicrobiaceae.

In one embodiment, the one or more gut microbiome species is within the Actinomycetaceae family and can be selected from one or more of the following: Actinomyces canis, Actinomyces cardiffensis, Actinomyces georgiae, Actinomyces graevenitzii, Actinomyces grossensis, Actinomyces naeslundii, Actinomyces odontolyticus, Actinomyces oris, Actinomyces radingae, Actinomyces turicensis, Actinomyces viscosus, Actinomyces urogenitalis, Arcanobacterium haemolyticum, Arcanobacterium pyogenes, Mobiluncus curtisii, Varibaculum cambriense, and Trueperella bernardiae.

In one embodiment, the one or more gut microbiome species is within the Bogoriellaceae family and can be Georgenia muralis.

In one embodiment, the one or more gut microbiome species is within the Brevibacteriaceae family and can be selected from one or more of the following: Brevibacterium casei, Brevibacterium epidermidis, Brevibacterium halotolerans, Brevibacterium iodinum, Brevibacterium linens, Brevibacterium massiliense, Brevibacterium pityocampae, Brevibacterium ravenspurgense, and Brevibacterium senegalense.

In one embodiment, the one or more gut microbiome species is within the Cellulomonadaceae family and can be selected from one or more of the following: Cellulomonas composti, Cellulomonas denverensis, Cellulomonas massiliensis, and Cellulomonas parahominis.

In one embodiment, the one or more gut microbiome species is within the Corynebacteriaceae family and can be selected from one or more of the following: Corynebacterium ammoniagenes, Corynebacterium afermentans, Corynebacterium amycolatum, Corynebacterium appendicis, Corynebacterium aurimucosum, Corynebacterium coyleae, Corynebacterium durum, Corynebacterium freneyi, Corynebacterium glaucum, Corynebacterium glucuronolyticum, Corynebacterium kroppenstedtii, Corynebacterium minutissimum, Corynebacterium mucifaciens, Corynebacterium propinquum, Corynebacterium pseudodiphthericum, Corynebacterium sanguinis, Corynebacterium simulans, Corynebacterium striatum, Corynebacterium sundsvallense, Corynebacterium tuberculostearicum, Corynebacterium ulcerans, Corynebacterium ureicelerivorans, and Corynebacterium xerosis.

In one embodiment, the one or more gut microbiome species is within the Dermabacteraceae family and can be selected from one or more of the following: Brachybacterium paraconglomeratum, Dermabacter hominis, Dermacoccus nishinomiyaensis, Kytococcus schroeteri, and Kytococcus sedentarius.

In one embodiment, the one or more gut microbiome species is within the Dietziaceae family and can be selected from one or more of the following: Dietzia cinnamea, Dietzia maris, and Dietzia natronolimnaea.

In one embodiment, the one or more gut microbiome species is within the Geodermatophilaceae family and can be Blastococcus massiliensis.

In one embodiment, the one or more gut microbiome species is within the Gordoniaceae family and can be selected from one or more of the following: Gordonia rubripertincta and Gordonia terrae.

In one embodiment, the one or more gut microbiome species is within the Intrasporangiaceae family and can be selected from one or more of the following: Janibacter limosus and Janibacter terrae.

In one embodiment, the one or more gut microbiome species is within the Microbacteriaceae family and can be selected from one or more of the following: Agrococcus jejuensis, Agrococcus terreus, Curtobacterium flaccumfaciens, Microbacterium aurum, Microbacterium chocolatum, Microbacterium foliorum, Microbacterium gubbeenense, Microbacterium hydrocarbonoxydans, Microbacterium lacticum, Microbacterium luteolum, Microbacterium oleivorans, Microbacterium paraoxydans, Microbacterium phyllosphaerae, Microbacterium schleiferi, Pseudoclavibacter massiliense, and Yonghaparkia alkaliphila.

In one embodiment, the one or more gut microbiome species is within the Micrococcaceae family and can be selected from one or more of the following: Arthrobacter albus, Arthrobacter castelli, Arthrobacter oxydans, Arthrobacter polychromogenes, Kocuria halotolerans, Kocuria kristinae, Kocuria marina, Kocuria palustris, Kocuria rhizophila, Kocuria rosea, Micrococcus luteus, Micrococcus lylae, Rothia aeria, Rothia dentocariosa, and Rothia mucilaginosa.

In one embodiment, the one or more gut microbiome species is within the Micromonosporaceae family and can be Micromonospora aurantiaca.

In one embodiment, the one or more gut microbiome species is within the Mycobacteriaceae family and can b selected from one or more of the following: Mycobacterium avium, Mycobacterium abscessus, Mycobacterium florentinum, and Mycobacterium fortuitum.

In one embodiment, the one or more gut microbiome species is within the Nocardiaceae family and can be selected from one or more of the following: Rhodococcus equi, Rhodococcus erythropolis, and Rhodococcus rhodochrous.

In one embodiment, the one or more gut microbiome species is within the Promicromonosporaceae family and can be selected from one or more of the following: Promicromonospora flava and Cellulosimicrobium cellulans.

In one embodiment, the one or more gut microbiome species is within the Propionibacterineae family and can be selected from one or more of the following: Aeromicrobium massiliense, Propionibacterium acidipropionici, Propionibacterium acnes, Propionibacterium avidum, Propionibacterium freudenreichii, Propionibacterium granulosum, Propionibacterium jensenii, and Propionibacterium propionicum.

In one embodiment, the one or more gut microbiome species is within the Streptomycetaceae family and can be selected from one or more of the following: Streptomyces massiliensis, Streptomyces misionensis, Streptomyces thermovulgaris, and Streptomyces thermoviolaceus.

In one embodiment, the one or more gut microbiome species is within the Micrococcineae family and can be selected from one or more of the following: Tropheryma whipplei and Timonella senegalensis.

In one embodiment, the one or more gut microbiome species is within the Bifidobacteriaceae family and can be selected from one or more of the following: Bifidobacterium adolescentis, Bifidobacterium angulatum, Bifidobacterium animalis, Bifidobacterium bifidum, Bifidobacterium boum, Bifidobacterium breve, Bifidobacterium catenulatum, Bifidobacterium coryneforme, Bifidobacterium dentium, Bifidobacterium gallicum, Bifidobacterium kashiwanohense, Bifidobacterium longum, Bifidobacterium mongoliense, Bifidobacterium pseudocatenulatum, Bifidobacterium pseudolongum, Bifidobacterium ruminantium, Bifidobacterium scardovii, Bifidobacterium stercoris, Bifidobacterium thermophilum, Bifidobacterium thermacidophilum, and Scardovia inopinata.

In one embodiment, the one or more gut microbiome species is within the Coriobacteriaceae family and can be selected from one or more of the following: Asaccharobacter celatus, Adlercreutzia equolifaciens, Atopobium minutum, Atopobium parvulum, Atopobium rimae, Collinsella aerofaciens, Collinsella intestinalis, Collinsella stercoris, Collinsella tanakaei, Cryptobacterium curtum, Eggerthella lenta, Enorma massiliensis, Gordonibacter pamelaeae, Olsenella profusa, Olsenella uli, Paraeggerthella hongkongensis, Senegalemassilia anaerobia, Slackia equolifaciens, Slackia exigua, Slackia isoflavoniconvertens, and Slackia piriformis.

In one embodiment, the one or more gut microbiome species is within the Deinococcaceae family and can be Deinococcus aquaticus.

In one embodiment, the one or more gut microbiome species is within the Halobacteroidaceae family and can be Halanaerobaculum tunisiense.

In one embodiment, the one or more gut microbiome species is within the Alicyclobacillaceae family and can be Tumebacillus permanentifrigoris.

In one embodiment, the one or more gut microbiome species is within the Bacillaceae family and can be selected from one or more of the following: Aeribacillus pallidus, Bacillus altitudinis, Bacillus amyloliquefaciens, Bacillus arsenicus, Bacillus atrophaeus, Bacillus badius, Bacillus beijingensis, Bacillus benzoevorans, Bacillus cereus, Bacillus circulans, Bacillus clausii, Bacillus endophyticus, Bacillus firmus, Bacillus flexus, Bacillus fordii, Bacillus halodurans, Bacillus idriensis, Bacillus infantis, Bacillus licheniformis, Bacillus marisflavi, Bacillus marseilloanorexicus, Bacillus massiliosenegalensis, Bacillus megaterium, Bacillus mojavensis, Bacillus mycoides, Bacillus nealsonii, Bacillus niacini, Bacillus polyfermenticus, Bacillus pseudofirmus, Bacillus pumilus, Bacillus schlegelii, Bacillus senegalensis, Bacillus simplex, Bacillus siralis, Bacillus sonorensis, Bacillus subtilis, Bacillus thermoamylovorans, Bacillus thuringiensis, Bacillus timonensis, Bacillus vallismortis, Geobacillus stearothermophilus, Geobacillus vulcani, Oceanobacillus caeni, Oceanobacillus massiliensis, and Virgibacillus proomii.

In one embodiment, the one or more gut microbiome species is within the Bacillales Family XI Incertae Sedis and can be selected from one or more of the following: Exiguobacterium aurantiacum, Gemella haemolysans, Gemella morbillorum, and Gemella sanguinis.

In one embodiment, the one or more gut microbiome species is within the Listeriaceae family and can be selected from Brochothrix thermosphacta.

In one embodiment, the one or more gut microbiome species is within the Paenibacillaceae family and can be selected from one or more of the following: Aneurinibacillus aneurinilyticus, Aneurinibacillus migulanus, Brevibacillus agri, Brevibacillus borstelensis, Brevibacillus brevis, Brevibacillus massiliensis, Paenibacillus alvei, Paenibacillus antibioticophila, Paenibacillus barcinonensis, Paenibacillus barengoltzii, Paenibacillus daejeonensis, Paenibacillus durus, Paenibacillus glucanolyticus, Paenibacillus graminis, Paenibacillus illinoisensis, Paenibacillus lactis, Paenibacillus lautus, Paenibacillus provencensis, Paenibacillus pueri, Paenibacillus rhizosphaerae, Paenibacillus senegalensis, Paenibacillus thiaminolyticus, and Paenibacillus timonensis.

In one embodiment, the one or more gut microbiome species is within the Planococcaceae family and can be selected from one or more of the following: Kurthia gibsonii, Kurthia massiliensis, Kurthia senegalensis, Kurthia timonensis, Lysinibacillus fusiformis, Lysinibacillus massiliensis, Lysinibacillus sphaericus, Planococcus rifietoensis, Planomicrobium chinense, Sporosarcina koreensis, Ureibacillus suwonensis, and Ureibacillus thermosphaericus.

In one embodiment, the one or more gut microbiome species is within the Staphylococcaceae family and can be selected from one or more of the following: Staphylococcus arlettae, Staphylococcus aureus, Staphylococcus auricularis, Staphylococcus capitis, Staphylococcus caprae, Staphylococcus cohnii, Staphylococcus condimenti, Staphylococcus epidermidis, Staphylococcus equorum, Staphylococcus haemolyticus, Staphylococcus hominis, Staphylococcus intermedius, Staphylococcus kloosii, Staphylococcus lugdunensis, Staphylococcus pasteuri, Staphylococcus pettenkoferi, Staphylococcus saccharolyticus, Staphylococcus saprophyticus, Staphylococcus schleiferi, Staphylococcus sciuri, Staphylococcus simulans, Staphylococcus succinus, Staphylococcus vitulinus, Staphylococcus warneri, and Staphylococcus xylosus.

In one embodiment, the one or more gut microbiome species is within the Aerococcaceae family and can be selected from one or more of the following: Abiotrophia defectiva, Abiotrophiapara-adiacens, Aerococcus viridans, and Facklamia tabacinasalis.

In one embodiment, the one or more gut microbiome species is within the Carnobacteriaceae family and can be selected from one or more of the following: Granulicatella adiacens and Granulicatella elegans.

In one embodiment, the one or more gut microbiome species is within the Enterococcaceae family and can be selected from one or more of the following: Enterococcus asini, Enterococcus avium, Enterococcus caccae, Enterococcus casseliflavus, Enterococcus cecorum, Enterococcus dispar, Enterococcus durans, Enterococcus faecalis, Enterococcus faecium, Enterococcus gallinarum, Enterococcus hirae, Enterococcus phoeniculicola, Enterococcus pseudoavium, Enterococcus saccharolyticus, and Tetragenococcus solitarius.

In one embodiment, the one or more gut microbiome species is within the Lactobacillaceae family and can be selected from one or more of the following: Lactobacillus acidophilus, Lactobacillus alimentarius, Lactobacillus amylovorus, Lactobacillus animalis, Lactobacillus antri, Lactobacillus brevis, Lactobacillus buchneri, Lactobacillus casei, Lactobacillus coleohominis, Lactobacillus coryniformis, Lactobacillus crispatus, Lactobacillus curvatus, Lactobacillus delbrueckii, Lactobacillus fermentum, Lactobacillus gasseri, Lactobacillus gastricus, Lactobacillus helveticus, Lactobacillus iners, Lactobacillus jensenii, Lactobacillus johnsonii, Lactobacillus kalixensis, Lactobacillus leichmanii, Lactobacillus mucosae, Lactobacillus oris, Lactobacillus parabuchneri, Lactobacillus paracasei, Lactobacillus pentosus, Lactobacillus plantarum, Lactobacillus reuteri, Lactobacillus rhamnosus, Lactobacillus ruminis, Lactobacillus sakei, Lactobacillus salivarius, Lactobacillus saniviri, Lactobacillus senioris, Lactobacillus sharpeae, Lactobacillus ultunensis, Lactobacillus vaginalis, Pediococcus acidilactici, Pediococcus damnosus, and Pediococcus pentosaceus.

In one embodiment, the one or more gut microbiome species is within the Leuconostocaceae family and can be selected from one or more of the following: Leuconostoc argentinium/lactis, Leuconostoc gelidum, Leuconostoc mesenteroides, Weissella cibaria, Weissella confusa, and Weissella paramesenteroides.

In one embodiment, the one or more gut microbiome species is within the Streptococcaceae family and can be selected from one or more of the following: Lactococcus garvieae, Lactococcus lactis, Lactococcus plantarum, Lactococcus raffinolactis, Streptococcus agalactiae, Streptococcus alactolyticus, Streptococcus anginosus, Streptococcus australis, Streptococcus bovis, Streptococcus constellatus, Streptococcus cristatus, Streptococcus dysgalactiae, Streptococcus equi, Streptococcus equinus, Streptococcus gallolyticus, Streptococcus gordonii, Streptococcus infantarius, Streptococcus infantis, Streptococcus intermedius, Streptococcus lutetiensis, Streptococcus mitis, Streptococcus mutans, Streptococcus oralis, Streptococcus parasanguinis, Streptococcus parauberis, Streptococcus peroris, Streptococcus pneumoniae, Streptococcus pseudopneumoniae, Streptococcus pyogenes, Streptococcus salivarius, Streptococcus sanguinis, Streptococcus thermophilus, Streptococcus thoraltensis, Streptococcus uberis, Streptococcus vestibularis, and Streptococcus viridans.

In one embodiment, the one or more gut microbiome species is within the Christensenellaceae family and can be selected from one or more of the following: Christensenella minuta and Catabacter hongkongensis.

In one embodiment, the one or more gut microbiome species is within the Clostridiaceae family and can be selected from one or more of the following: Clostridium acetobutylicum, Clostridium anorexicamassiliense, Clostridium asparagiforme, Clostridium baratii, Clostridium beijerinckii, Clostridium botulinum, Clostridium butyricum, Clostridium cadaveris, Clostridium celatum, Clostridium chartatabidum, Clostridium chauvoei, Clostridium cochlearium, Clostridium disporicum, Clostridium fallax, Clostridium felsineum, Clostridium limosum, Clostridium malenominatum, Clostridium neonatale, Clostridium paraputrificum, Clostridium perfringens, Clostridium putrefaciens, Clostridium saccharoperbutylacetonicum, Clostridium sardiniense, Clostridium sartagoforme, Clostridium scindens, Clostridium senegalense, Clostridium septicum, Clostridium sporogenes, Clostridium subterminale, Clostridium tertium, Clostridium tyrobutyricum, Clostridium vincentii, Eubacterium budayi, Eubacterium hallii, Eubacterium moniliforme, Eubacterium multiforme, Eubacterium nitritogenes, Sarcina maxima, and Sarcina ventriculi.

In one embodiment, the one or more gut microbiome species is within the Ruminococcaceae family and can be selected from one or more of the following: Acetanaerobacterium elongatum, Anaerofilum pentosovorans, Anaerotruncus colihominis, Butyricicoccus pullicaecorum, Clostridium anorexicus (Intestinimonas butyriciproducens), Clostridium cellobioparum, Clostridium clariflavum, Clostridium leptum, Clostridium methylpentosum, Clostridium sporosphaeroides, Clostridium viride, Eubacterium desmolans, Eubacterium siraeum, Faecalibacterium prausnitzii, Flavonifractor plautii, Gemmiger formicilis, Hydrogenoanaerobacterium saccharovorans, Oscillibacter valericigenes, Papillibacter cinnamivorans, Pseudoflavonifractor capillosus, Ruminococcus albus, Ruminococcus bromii, Ruminococcus callidus, Ruminococcus champanellensis, Ruminococcus flavefaciens, Ruminococcus lactaris, Ruminococcus torques, Soleaferrea massiliensis, Subdoligranulum variabile, Anaerotruncus unclassified, and Subdoligranulum unclassified.

In one embodiment, the one or more gut microbiome species is within the Clostridiales Family XIII Incertae Sedis and can be selected from one or more of the following: Eubacterium brachy, Eubacterium saphenum, Eubacterium siraeum, Eubacterium sulci, Mogibacterium diversum, Mogibacterium neglectum, Mogibacterium timidum, and Mogibacterium vescum.

In one embodiment, the one or more gut microbiome species is within the Peptostreptococcaceae family and can be selected from one or more of the following: Anoxynatronum sibiricum, Clostridium difficile, Clostridium bartlettii, Clostridium bifermentans, Clostridium ghonii, Clostridium glycolicum, Clostridium hiranonis, Clostridium irregulare, Clostridium lituseburense, Clostridium sordellii, Clostridium sticklandii, Eubacterium tenue, Filifactor alocis, Filifactor villosus, Peptostreptococcus anaerobius, and Peptostreptococcus stomatis.

In one embodiment, the one or more gut microbiome species is within the Clostridiales Family XIIncertae Sedis and can be selected from one or more of the following: Anaerococcus hydrogenalis, Anaerococcus obesiensis, Anaerococcus octavius, Anaerococcus prevotii, Anaerococcus senegalensis, Anaerococcus vaginalis, Bacteroides coagulans, Finegoldia magna, Kallipyga massiliensis, Parvimonas micra, Peptoniphilus asaccharolyticus, Peptoniphilus grossensis, Peptoniphilus harei, Peptoniphilus indolicus, Peptoniphilus lacrimalis, Peptoniphilus obesiensis, Peptoniphilus senegalensis, Peptoniphilus timonensis, and Tissierella praeacuta.

In one embodiment, the one or more gut microbiome species is within the Lachnospiraceae family and can be selected from one or more of the following: Anaerostipes butyraticus, Anaerostipes caccae, Anaerostipes coli, Anaerostipes rhamnosus, Anaerostipes hadrus, Anoxystipes contortum, Anoxystipes fissicatena, Anoxystipes oroticum, Bacteroides pectinophilus, Blautia coccoides, Blautiafaecis, Blautia glucerasea, Blautia hansenii, Blautia hydrogenotrophica, Blautia luti, Blautia (Ruminococcus) massiliensis, Blautia (Ruminococcus) obeum, Blautiaproducta, Blautia stercoris, Blautia wexlerae, Butyrivibrio crossotus, Butyrivibrio fibrisolvens, Cellulosilyticum lentocellum, Clostridium aminovalericum, Clostridium aldenense, Clostridium asparagiforme, Clostridium bolteae, Clostridium citroniae, Clostridium clostridioforme, Clostridium glycyrrhizinilyticum, Clostridium hathewayi, Clostridium herbivorans, Clostridium hylemonae, Clostridium indolis, Clostridium lactatifermentans, Clostridium lavalense, Clostridium methoxybenzovorans, Clostridium nexile, Clostridium populeti, Clostridium scindens, Clostridium sphenoides, Clostridium symbiosum, Coprococcus catus, Coprococcus comes, Coprococcus eutactus, Doreaformicigenerans, Dorea longicatena, Dorea massiliensis, Eubacterium cellulosolvens, Eubacterium eligens, Eubacterium hallii, Eubacterium ramulus, Eubacterium rectale, Eubacterium ruminantium, Eubacterium ventriosum, Fusicatenibacter saccharivorans, Hespellia porcina, Hespellia stercorisuis, Howardella ureilytica, Lachnoanaerobaculum saburreum, Lachnoanaerobaculum umeaense, Bacteroides galacturonicus, Lachnospira pectinoschiza, Lactobacillus rogosae, Lactonifactor longoviformis, Lachnobacterium bovis, Marvinbryantiaformatexigens, Moryella indoligenes, Oribacterium sinus, Parasporobacterium paucivorans, Robinsoniella peoriensis, Roseburia faecis, Roseburia hominis, Roseburia intestinalis, Roseburia inulinivorans, Ruminococcus gauvreauii, Ruminococcus gnavus, Ruminococcusfaecis, Ruminococcus lactaris, Ruminococcus torques, Lachnospiracea bacterium 5_1_63FAA, Lachnospiraceae bacterium 3_1_57FAA_CT1, and Lachnospiraceae bacterium 8_1_57FAA.

In one embodiment, the one or more gut microbiome species is within the Eubacteriaceae family and can be selected from one or more of the following: Anaerofustis stercorihominis, Eubacterium barkeri, Eubacterium callanderi, Eubacterium limosum, and Pseudoramibacter alactolyticus.

In one embodiment, the one or more gut microbiome species is within the Erysipelotrichaceae family and can be Turicibacter sanguinis.

In one embodiment, the one or more gut microbiome species is within the Erysipelotrichaceae XVI family and can be selected from one or more of the following: Clostridium innocuum, Eubacterium biforme, Eubacterium cylindroides, Eubacterium dolichum, Eubacterium tortuosum, Dielma fastidiosa, and Streptococcus pleomorphus.

In one embodiment, the one or more gut microbiome species is within the Erysipelotrichaceae XVII and can be selected from one or more of the following: Catenibacterium mitsuokai, Coprobacillus cateniformis, Coprobacillus unclassified, Eggerthia catenaformis, Kandleria vitulina, and Stoquefichus massiliensis.

In one embodiment, the one or more gut microbiome species is within the Erysipelotrichaceae XVIII and can be selected from one or more of the following: Anaerorhabdus furcosa, Bulleidia extructa, Clostridium cocleatum, Clostridium ramosum, Clostridium saccharogumia, Clostridium spiroforme, Clostridium symbiosum, Holdemania filiformis, Holdemania massiliensis, and Solobacterium moorei.

In one embodiment, the one or more gut microbiome species is within the Acidiaminococcaceae family and can be selected from one or more of the following: Acidaminococcus fermentans, Acidaminococcus intestini, Phascolarctobacterium faecium, and Phascolarctobacterium succinatutens.

In one embodiment, the one or more gut microbiome species is within the Peptococcaceae family and can be selected from one or more of the following: Peptococcus niger and Desulfitobacterium frappieri.

In one embodiment, the one or more gut microbiome species is within the Veillonellaceae family and can be selected from one or more of the following: Allisonella histaminiformans, Dialister invisus, Dialister pneumosintes, Dialister succinatiphilus, Megamonas funiformis, Megamonas hypermegale, Megasphaera elsdenii, Mitsuokella jalaludinii, Mitsuokella multacida, Negativicoccus succinicivorans, Selenomonas ruminantium, Veillonella atypica, Veillonella dispar, Veillonella parvula, Veillonella ratti, Veillonella rogosae, and Veillonella unclassified.

In one embodiment, the one or more gut microbiome species is within the Bacteroidaceae family and can be selected from one or more of the following: Bacteroides caccae, Bacteroides cellulosilyticus, Bacteroides clarus, Bacteroides coprocola, Bacteroides coprophilus, Bacteroides dorei, Bacteroides faecis, Bacteroides eggerthii, Bacteroides finegoldii, Bacteroides fluxus, Bacteroides fragilis, Bacteroides graminisolvens, Bacteroides intestinalis, Bacteroides massiliensis, Bacteroides nordii, Bacteroides oleiciplenus, Bacteroides ovatus, Bacteroides plebeius, Bacteroides pyogenes, Bacteroides salyersiae, Bacteroides stercoris, Bacteroides thetaiotaomicron, Bacteroides timonensis, Bacteroides uniformis, Bacteroides vulgatus, Bacteroides xylanisolvens, and Bacteroidales ph8.

In one embodiment, the one or more gut microbiome species is within the Porphyromonadaceae family and can be selected from one or more of the following: Barnesiella intestinihominis, Butyricimonas synergistica, Butyricimonas virosa, Dysgonomonas gadei, Odoribacter laneus, Odoribacter splanchnicus, Parabacteroides distasonis, Parabacteroides goldsteinii, Parabacteroides gordonii, Parabacteroides johnsonii, Parabacteroides merdae, Porphyromonas asaccharolytica, Porphyromonas endodontalis, Porphyromonas gingivalis, Porphyromonas somerae, Porphyromonas uenonis, and Tannerella forsythia.

In one embodiment, the one or more gut microbiome species is within the Prevotellaceae and can be selected from one or more of the following: Barnesiella intestinihominis, Alloprevotella tannerae, Prevotella albensis, Prevotella amniotica, Prevotella bivia, Prevotella brevis, Prevotella buccae, Prevotella bryantii, Prevotella conceptionensis, Prevotella copri, Prevotella corporis, Prevotella denticola, Prevotella disiens, Prevotella enoeca, Prevotella intermedia, Prevotella loescheii, Prevotella melaninogenica, Prevotella nanceiensis, Prevotella nigrescens, Prevotella oulora, Prevotella oralis, Prevotella pallens, Prevotella ruminicola, Prevotella shahii, Prevotella stercorea, Prevotella timonensis, Prevotella veroralis, Paraprevotella clara, Paraprevotella xylaniphila, and Paraprevotella unclassified.

In one embodiment, the one or more gut microbiome species is within the Rikenellaceae family and can be selected from one or more of the following: Alistipes finegoldii, Alistipes indistinctus, Alistipes marseilloanorexicus, Alistipes obesi, Alistipes onderdonkii, Alistipes putredinis, Alistipes senegalensis, Alistipes shahii, and Alistipes timonensis.

In one embodiment, the one or more gut microbiome species is within the Cytophagaceae family and can be selected from one or more of the following: Dyadobacter beijingensis, Dyadobacter fermentans, Hymenobacter rigui, Rudanella lutea, and Spirosoma linguale.

In one embodiment, the one or more gut microbiome species is within the Flavobacteriaceae family and can be selected from one or more of the following: Capnocytophaga granulosa, Capnocytophaga ochracea, Capnocytophaga sputigena, Chryseobacterium hominis, Cloacibacterium normanense, Flavobacterium banpakuense, Flavobacterium cheniae, Flavobacterium lindanitolerans, Flavobacterium oncorhynchi, Flavobacterium sakaeratica, and Wautersiella falsenii.

In one embodiment, the one or more gut microbiome species is within the Chitinophagaceae family and can be Bifissio spartinae.

In one embodiment, the one or more gut microbiome species is within the Sphingobacteriaceae family and can be selected from one or more of the following: Sphingobacterium multivorum and Pedobacter daejeonensis.

In one embodiment, the one or more gut microbiome species is within the Fusobacteriaceae family and can be selected from one or more of the following: Cetobacterium somerae, Clostridium rectum, Fusobacterium gonidiaformans, Fusobacterium mortiferum, Fusobacterium naviforme, Fusobacterium necrogenes, Fusobacterium necrophorum, Fusobacterium nucleatum, Fusobacterium periodonticum, Fusobacterium russii, and Fusobacterium varium.

In one embodiment, the one or more gut microbiome species is within the Leptotrichiaceae family and can be selected from one or more of the following: Leptotrichia amnionii and Leptotrichia buccalis.

In one embodiment, the one or more gut microbiome species is within the Victivallaceae family and can be Victivallis vadensis.

In one embodiment, the one or more gut microbiome species is within the Planctomycetaceae family and can be Schlesneria paludicola.

In one embodiment, the one or more gut microbiome species is within the Caulobacteraceae family and can be selected from one or more of the following: Brevundimonas bacteroides, Brevundimonas diminuta, Brevundimonas terrae, Brevundimonas vesicularis, and Phenylobacterium haematophilum.

In one embodiment, the one or more gut microbiome species is within the Aurantimonadaceae family and can be Aurantimonas altamirensis.

In one embodiment, the one or more gut microbiome species is within the Bradyrhizobiaceae family and can be selected from one or more of the following: Bradyrhizobium denitrificans, Bradyrhizobium elkanii, Bradyrhizobium japonicum, and Afipia birgiae.

In one embodiment, the one or more gut microbiome species is within the Brucellaceae family and can be selected from one or more of the following: Ochrobactrum anthropi and Ochrobactrum intermedium.

In one embodiment, the one or more gut microbiome species is within the Hyphomicrobiaceae and can be Pedomicrobium ferrugineum.

In one embodiment, the one or more gut microbiome species is within the Methylobacteriaceae family and can be selected from one or more of the following: Methylobacterium adhaesivum, Methylobacterium jeotgali, Methylobacterium mesophilicum, Methylobacterium populi, Methylobacterium radiotolerans, Methylobacterium zatmanii, and Microvirga massiliensis.

In one embodiment, the one or more gut microbiome species is within the Phyllobacteriaceae family and can be selected from one or more of the following: Mesorhizobium loti and Phyllobacterium myrsinacearum.

In one embodiment, the one or more gut microbiome species is within the Rhizobiaceae family and can be Agrobacterium tumefaciens.

In one embodiment, the one or more gut microbiome species is within the Xanthobacteraceae family and can be Ancylobacter polymorphus.

In one embodiment, the one or more gut microbiome species is within the Rhodobacteraceae family and can be selected from one or more of the following: Paracoccus carotinifaciens, Paracoccus marinus, Paracoccus yeei, and Amaricoccus kaplicensis.

In one embodiment, the one or more gut microbiome species is within the Acetobacteraceae family and can be Roseomonas mucosa.

In one embodiment, the one or more gut microbiome species is within the Rhodospirillaceae family and can be Skermanella aerolata.

In one embodiment, the one or more gut microbiome species is within the Sphingomonadaceae family and can be selected from one or more of the following: Blastomonas natatoria, Sphingomonas panni, Sphingomonas pseudosanguinis, Sphingomonas paucimobilis, and Sphingomonas adhaesiva.

In one embodiment, the one or more gut microbiome species is within the Alcaligenaceae family and can be selected from one or more of the following: Achromobacter denitrificans, Achromobacter xylosoxidans, Alcaligenes faecalis, Bordetella hinzii, and Kerstersia gyiorum.

In one embodiment, the one or more gut microbiome species is within the Burkholderiaceae family and can be selected from one or more of the following: Burkholderia cepacia, Cupriavidus metallidurans, Lautropia mirabilis, Limnobacter thiooxidans, and Ralstonia mannitolilytica.

In one embodiment, the one or more gut microbiome species is within the Comamonadaceae family and can be selected from one or more of the following: Acidovorax facilis, Aquabacterium commune, Comamonas kerstersii, Comamonas testosteroni, Delftia acidovorans, Pelomonas saccharophila, and Variovorax boronicumulans.

In one embodiment, the one or more gut microbiome species is within the Oxalobacteraceae family and can be selected from one or more of the following: Herbaspirillum massiliense, Massilia aurea, and Oxalobacter formigenes.

In one embodiment, the one or more gut microbiome species is within the Suterellaceae family and can be selected from one or more of the following: Parasutterella excrementihominis, Parasutterella secunda, Sutterella parvirubra, Sutterella stercoricanis, and Sutterella wadsworthensis.

In one embodiment, the one or more gut microbiome species is within the Neisseriaceae family and can be selected from one or more of the following: Eikenella corrodens, Laribacter hongkongensis, Kingella oralis, Neisseria cinerea, Neisseria elongata, Neisseriaflava, Neisseria flavescens, Neisseria macacae, Neisseria mucosa, Neisseria perflava, and Neisseria subflava.

In one embodiment, the one or more gut microbiome species is within the Rhodocyclaceae family and can be Methyloversatilis universalis.

In one embodiment, the one or more gut microbiome species is within the Desulfovibrionaceae family and can be selected from one or more of the following: Desulfovibrio desulfuricans, Desulfovibrio fairfieldensis, Desulfovibrio piger, and Bilophila wadsworthia.

In one embodiment, the one or more gut microbiome species is within the Campylobacteraceae family and can be selected from one or more of the following: Arcobacter butzleri, Arcobacter cryaerophilus, Bacteroides ureolyticus, Campylobacter coli, Campylobacter concisus, Campylobacter curvus, Campylobacter faecalis, Campylobacter fetus, Campylobacter gracilis, Campylobacter hominis, Campylobacter hyointestinalis, Campylobacterjejuni, Campylobacter lari, Campylobacter rectus, Campylobacter showae, and Campylobacter upsaliensis.

In one embodiment, the one or more gut microbiome species is within the Helicobacteraceae family and can be selected from one or more of the following: Flexispira rappini, Helicobacter canadensis, Helicobacter cinaedi, Helicobacter pullorum, Helicobacter pylori, and Helicobacter winghamensis.

In one embodiment, the one or more gut microbiome species is within the Aeromonadaceae family and can be selected from one or more of the following: Aeromonas allosaccharophila, Aeromonas bestiarum, Aeromonas caviae, Aeromonas enteropelogenes, Aeromonas hydrophila, Aeromonas jandaei, Aeromonas media, Aeromonas tecta, Aeromonas trota, and Aeromonas veronii.

In one embodiment, the one or more gut microbiome species is within the Succinivibrionaceae family and can be selected from one or more of the following: Anaerobiospirillum thomasii, Anaerobiospirillum succiniciproducens, Succinatimonas hippei, and Succinivibrio dextrinosolvens.

In one embodiment, the one or more gut microbiome species is within the Enterobacteriaceae family and can be selected from one or more of the following: Averyella dalhousiensis, Cedecea davisae, Citrobacter amalonaticus, Citrobacter braakii, Citrobacter farmeri, Citrobacter intermedius, Citrobacter koseri, Citrobacter freundii, Citrobacter gillenii, Citrobacter murliniae, Citrobacter sedlakii, Citrobacter werkmanii, Citrobacter youngae, Cronobacter sakazakii, Edwardsiella tarda, Enterobacter aerogenes, Enterobacter asburiae, Enterobacter cancerogenus, Enterobacter cloacae, Enterobacter hormaechei, Enterobacter ludwigii, Enterobacter massiliensis, Escherichia albertii, Escherichia coli, Escherichia fergusonii, Escherichia hermannii, Hafnia alvei, Klebsiella oxytoca, Klebsiella pneumoniae, Kluyvera ascorbata, Leminorella grimontii, Leminorella richardii, Moellerella wisconsensis, Morganella morganii, Pantoea agglomerans, Plesiomonas shigelloides, Proteus mirabilis, Proteus penneri, Proteus vulgaris, Providencia alcalifaciens, Providencia rettgeri, Providencia rustigianii, Providencia stuartii, Raoultella planticola, Raoultella terrigena, Salmonella enterica, Serratia ficaria, Serratia fonticola, Serratia liquefaciens, Serratia marcescens, Shigella dysenteriae, Shigella flexneri, Shigella sonnei, Tatumella ptyseos, Trabulsiella guamensis, Yersinia aleksiciae, Yersinia bercovieri, Yersinia enterocolitica, Yersinia frederiksenii, Yersinia kristensenii, Yersinia pseudotuberculosis, Yersinia rohdei, and Yokenella regensburgei.

In one embodiment, the one or more gut microbiome species is within the Pasteurellaceae family and can be selected from one or more of the following: Actinobacillus pleuropneumoniae, Aggregatibacter aphrophilus, Haemophilus haemolyticus, Haemophilus influenzae, Haemophilus parainfluenzae, Haemophilus quentini, and Haemophilus sputorum.

In one embodiment, the one or more gut microbiome species is within the Moraxellaceae family and can be selected from one or more of the following: Acinetobacter baumannii, Acinetobacter calcoaceticus, Acinetobacter haemolyticus, Acinetobacter johnsonii, Acinetobacter junii, Acinetobacter lwoffii, Acinetobacter pittii, Acinetobacter radioresistens, Acinetobacter septicus, Moraxella catarrhalis, Moraxella osloensis, and Psychrobacter arenosus.

In one embodiment, the one or more gut microbiome species is within the Pseudomonadaceae family and can be selected from one or more of the following: Pseudomonas alcaliphila, Pseudomonas aeruginosa, Pseudomonas fluorescens, Pseudomonas monteilii, Pseudomonas nitroreducens, Pseudomonas oleovorans, Pseudomonas putida, and Pseudomonas stutzeri.

In one embodiment, the one or more gut microbiome species is within Vibrionaceae family and can be selected from one or more of the following: Grimontia hollisae, Vibrio fluvialis, Vibrio furnissii, Vibrio mimicus, and Vibrio parahaemolyticus.

In one embodiment, the one or more gut microbiome species is within the Sinobacteraceae family and can be Nevskia ramosa.

In one embodiment, the one or more gut microbiome species is within the Xanthomnonadaceae family and can be selected from one or more of the following: Lysobacter soli, Pseudoxanthomonas mexicana, Rhodanobacter ginsenosidimutans, Silanimonas lenta, Stenotrophomonas maltophilia, and Stenotrophomonas rhizophila.

In one embodiment, the one or more gut microbiome species is within the Brachyspiraceae family and can be selected from one or more of the following: Brachyspira aalborgi and Brachyspira pilosicoli.

In one embodiment, the one or more gut microbiome species is within the Synergistaceae family and can be selected from one or more of the following: Cloacibacillus evryensis and Pyramnidobacter piscolens.

In one embodiment, the one or more gut microbiome species is within the Mycoplasnataceae family and can be selected from one or more of the following: Mycoplasma pneumoniae, Mycoplasma hominis, Ureaplasma urealyticum, and Ureaplasma parvum

In one embodiment, the one or more gut microbiome species is within the Verrucomicrobiaceae family and can be selected from one or more of the following: Prosthecobacter fluviatilis and Akkermansia muciniphila.

In one embodiment, the one or more gut microbiome species is gram positive, selected from a family of:

Bifidobacteriaceae, selected from Bifidobacterium adolescentis, Bifidobacterium angulatum, Bifidobacterium animalis, Bifidobacterium bifidum, Bifidobacterium boum, Bifidobacterium breve, Bifidobacterium catenulatum, Bifidobacterium coryneforme, Bifidobacterium dentium, Bifidobacterium gallicum, Bifidobacterium kashiwanohense, Bifidobacterium longum, Bifidobacterium mongoliense, Bifidobacterium pseudocatenulatum, Bifidobacterium pseudolongum, Bifidobacterium ruminantium, Bifidobacterium scardovii, Bifidobacterium stercoris, Bifidobacterium thermophilum, Bifidobacterium thermacidophilum, and Scardovia inopinata,

Lactobacillaceae, selected from Lactobacillus acidophilus, Lactobacillus alimentarius, Lactobacillus amylovorus, Lactobacillus animalis, Lactobacillus antri, Lactobacillus brevis, Lactobacillus buchneri, Lactobacillus casei, Lactobacillus coleohominis, Lactobacillus coryniformis, Lactobacillus crispatus, Lactobacillus curvatus, Lactobacillus delbrueckii, Lactobacillus fermentum, Lactobacillus gasseri, Lactobacillus gastricus, Lactobacillus helveticus, Lactobacillus iners, Lactobacillus jensenii, Lactobacillus johnsonii, Lactobacillus kalixensis, Lactobacillus leichmanii, Lactobacillus mucosae, Lactobacillus oris, Lactobacillus parabuchneri, Lactobacillus paracasei, Lactobacillus pentosus, Lactobacillus plantarum, Lactobacillus reuteri, Lactobacillus rhamnosus, Lactobacillus ruminis, Lactobacillus sakei, Lactobacillus salivarius, Lactobacillus saniviri, Lactobacillus senioris, Lactobacillus sharpeae, Lactobacillus ultunensis, Lactobacillus vaginalis, Pediococcus acidilactici, Pediococcus damnosus, and Pediococcus pentosaceus,

Streptococcaceae, selected from Lactococcus garvieae, Lactococcus lactis, Lactococcus plantarum, Lactococcus raffinolactis, Streptococcus agalactiae, Streptococcus alactolyticus, Streptococcus anginosus, Streptococcus australis, Streptococcus bovis, Streptococcus constellatus, Streptococcus cristatus, Streptococcus dysgalactiae, Streptococcus equi, Streptococcus equinus, Streptococcus gallolyticus, Streptococcus gordonii, Streptococcus infantarius, Streptococcus infantis, Streptococcus intermedius, Streptococcus lutetiensis, Streptococcus mitis, Streptococcus mutans, Streptococcus oralis, Streptococcus parasanguinis, Streptococcus parauberis, Streptococcus peroris, Streptococcus pneumoniae, Streptococcus pseudopneumoniae, Streptococcus pyogenes, Streptococcus salivarius, Streptococcus sanguinis, Streptococcus thermophilus, Streptococcus thoraltensis, Streptococcus uberis, Streptococcus vestibularis, and Streptococcus viridans,

Ruminococcaceae, selected from Acetanaerobacterium elongatum, Anaerofilum pentosovorans, Anaerotruncus colihominis, Butyricicoccus pullicaecorum, Clostridium anorexicus (Intestinimonas butyriciproducens), Clostridium cellobioparum, Clostridium clariflavum, Clostridium leptum, Clostridium methylpentosum, Clostridium sporosphaeroides, Clostridium viride, Eubacterium desmolans, Eubacterium siraeum, Faecalibacterium prausnitzii, Flavonifractor plautii, Gemmiger formicilis, Hydrogenoanaerobacterium saccharovorans, Oscillibacter valericigenes, Papillibacter cinnamivorans, Pseudoflavonifractor capillosus, Ruminococcus albus, Ruminococcus bromii, Ruminococcus callidus, Ruminococcus champanellensis, Ruminococcus flavefaciens, Ruminococcus lactaris, Ruminococcus torques, Soleaferrea massiliensis, Subdoligranulum variabile, Anaerotruncus unclassified, and Subdoligranulum unclassified,

Peptostreptococcaceae, selected from Anoxynatronum sibiricum, Clostridium difficile, Clostridium bartlettii, Clostridium bifermentans, Clostridium ghonii, Clostridium glycolicum, Clostridium hiranonis, Clostridium irregulare, Clostridium lituseburense, Clostridium sordellii, Clostridium sticklandii, Eubacterium tenue, Filifactor alocis, Filifactor villosus, Peptostreptococcus anaerobius, and Peptostreptococcus stomati,

Lachnospiraceae, selected from Anaerostipes butyraticus, Anaerostipes caccae, Anaerostipes coli, Anaerostipes rhamnosus, Anaerostipes hadrus, Anoxystipes contortum, Anoxystipes fissicatena, Anoxystipes oroticum, Bacteroides pectinophilus, Blautia coccoides, Blautia faecis, Blautia glucerasea, Blautia hansenii, Blautia hydrogenotrophica, Blautia luti, Blautia (Ruminococcus) massiliensis, Blautia (Ruminococcus) obeum, Blautia producta, Blautia stercoris, Blautia wexlerae, Butyrivibrio crossotus, Butyrivibrio fibrisolvens, Cellulosilyticum lentocellum, Clostridium aminovalericum, Clostridium aldenense, Clostridium asparagiforme, Clostridium bolteae, Clostridium citroniae, Clostridium clostridioforme, Clostridium glycyrrhizinilyticum, Clostridium hathewayi, Clostridium herbivorans, Clostridium hylemonae, Clostridium indolis, Clostridium lactatifermentans, Clostridium lavalense, Clostridium methoxybenzovorans, Clostridium nexile, Clostridium populeti, Clostridium scindens, Clostridium sphenoides, Clostridium symbiosum, Coprococcus catus, Coprococcus comes, Coprococcus eutactus, Doreaformicigenerans, Dorea longicatena, Dorea massiliensis, Eubacterium cellulosolvens, Eubacterium eligens, Eubacterium hallii, Eubacterium ramulus, Eubacterium rectale, Eubacterium ruminantium, Eubacterium ventriosum, Fusicatenibacter saccharivorans, Hespellia porcina, Hespellia stercorisuis, Howardella ureilytica, Lachnoanaerobaculum saburreum, Lachnoanaerobaculum umeaense, Bacteroides galacturonicus, Lachnospira pectinoschiza, Lactobacillus rogosae, Lactonifactor longoviformis, Lachnobacterium bovis, Marvinbryantia formatexigens, Moryella indoligenes, Oribacterium sinus, Parasporobacterium paucivorans, Robinsoniella peoriensis, Roseburia faecis, Roseburia hominis, Roseburia intestinalis, Roseburia inulinivorans, Ruminococcus gauvreauii, Ruminococcus gnavus, Ruminococcusfaecis, Ruminococcus lactaris, Ruminococcus torques, Lachnospiracea bacterium 5_1_63FAA, Lachnospiraceae bacterium 3_1_57FAA_CT1, and Lachnospiraceae bacterium 8_1_57FAA,

Erysipelotrichaceae XVII, selected from Catenibacterium mitsuokai, Coprobacillus cateniformis, Coprobacillus unclassified, Eggerthia catenaformis, Kandleria vitulina, and Stoquefichus massiliensis,

Erysipelotrichaceae XVIII, selected from Anaerorhabdus furcosa, Bulleidia extructa, Clostridium cocleatum, Clostridium ramosum, Clostridium saccharogumia, Clostridium spiroforme, Clostridium symbiosum, Holdemania filiformis, Holdemania massiliensis, and Solobacterium moorei, and

Veillonellaceae, selected from Allisonella histaminiformans, Dialister invisus, Dialister pneumosintes, Dialister succinatiphilus, Megamonas funiformis, Megamonas hypermegale, Megasphaera elsdenii, Mitsuokella jalaludinii, Mitsuokella multacida, Negativicoccus succinicivorans, Selenomonas ruminantium, Veillonella atypica, Veillonella dispar, Veillonella parvula, Veillonella ratti, Veillonella rogosae, and Veillonella unclassified.

In one embodiment, the one or more gut microbiome species is selected from Bifidobacterium breve, Bifidobacterium longum, Lactobacillus casei, Lactobacillus paracasei, Pediococcus pentosaceus, Lactococcus lactis, Streptococcus parasanguinis, Streptococcus salivarius, Streptococcus thermophilus, Ruminococcus bromii, Ruminococcus torques, Anaerotruncus unclassified, Subdoligranulum unclassified, Clostridium difficile, Blautia (Ruminococcus) obeum, Dorea longicatena, Eubacterium ramulus, Ruminococcus gnavus, Ruminococcus torques, Lachnospiracea bacterium 5_1_63FAA, Lachnospiraceae bacterium 3_1_57FAA_CT1, and Lachnospiraceae bacterium 8_1_57FAA, Coprobacillus unclassified, Clostridium spiroforme, Clostridium symbiosum, Veillonella parvula, and Veillonella unclassified.

In one embodiment, the one or more gut microbiome species is gram negative, selected from a family of:

Bacteroidaceae, selected from Bacteroides caccae, Bacteroides cellulosilyticus, Bacteroides clarus, Bacteroides coprocola, Bacteroides coprophilus, Bacteroides dorei, Bacteroides faecis, Bacteroides eggerthii, Bacteroides finegoldii, Bacteroides fluxus, Bacteroidesfragilis, Bacteroides graminisolvens, Bacteroides intestinalis, Bacteroides massiliensis, Bacteroides nordii, Bacteroides oleiciplenus, Bacteroides ovatus, Bacteroides plebeius, Bacteroides pyogenes, Bacteroides salyersiae, Bacteroides stercoris, Bacteroides thetaiotaomicron, Bacteroides timonensis, Bacteroides uniformis, Bacteroides vulgatus, Bacteroides xylanisolvens, and Bacteroidales ph8,

Porphyromonadaceae, selected from Barnesiella intestinihominis, Butyricimonas synergistica, Butyricimonas virosa, Dysgonomonas gadei, Odoribacter laneus, Odoribacter splanchnicus, Parabacteroides distasonis, Parabacteroides goldsteinii, Parabacteroides gordonii, Parabacteroides johnsonii, Parabacteroides merdae, Porphyromonas asaccharolytica, Porphyromonas endodontalis, Porphyromonas gingivalis, Porphyromonas somerae, Porphyromonas uenonis, and Tannerella forsythia,

Prevotellaceae, selected from Barnesiella intestinihominis, Alloprevotella tannerae, Prevotella albensis, Prevotella amniotica, Prevotella bivia, Prevotella brevis, Prevotella buccae, Prevotella bryantii, Prevotella conceptionensis, Prevotella copri, Prevotella corporis, Prevotella denticola, Prevotella disiens, Prevotella enoeca, Prevotella intermedia, Prevotella loescheii, Prevotella melaninogenica, Prevotella nanceiensis, Prevotella nigrescens, Prevotella oulora, Prevotella oralis, Prevotella pallens, Prevotella ruminicola, Prevotella shahii, Prevotella stercorea, Prevotella timonensis, Prevotella veroralis, Paraprevotella clara, Paraprevotella xylaniphila, and Paraprevotella unclassified,

Rikenellaceae, selected from Alistipes finegoldii, Alistipes indistinctus, Alistipes marseilloanorexicus, Alistipes obesi, Alistipes onderdonkii, Alistipes putredinis, Alistipes senegalensis, Alistipes shahii, and Alistipes timonensis,

Enterobacteriaceae, selected from Averyella dalhousiensis, Cedecea davisae, Citrobacter amalonaticus, Citrobacter braakii, Citrobacterfarmeri, Citrobacter intermedius, Citrobacter koseri, Citrobacter freundii, Citrobacter gillenii, Citrobacter murliniae, Citrobacter sedlakii, Citrobacter werkmanii, Citrobacter youngae, Cronobacter sakazakii, Edwardsiella tarda, Enterobacter aerogenes, Enterobacter asburiae, Enterobacter cancerogenus, Enterobacter cloacae, Enterobacter hormaechei, Enterobacter ludwigii, Enterobacter massiliensis, Escherichia albertii, Escherichia coli, Escherichia fergusonii, Escherichia hermannii, Hafnia alvei, Klebsiella oxytoca, Klebsiella pneumoniae, Kluyvera ascorbata, Leminorella grimontii, Leminorella richardii, Moellerella wisconsensis, Morganella morganii, Pantoea agglomerans, Plesiomonas shigelloides, Proteus mirabilis, Proteus penneri, Proteus vulgaris, Providencia alcalifaciens, Providencia rettgeri, Providencia rustigianii, Providencia stuartii, Raoultella planticola, Raoultella terrigena, Salmonella enterica, Serratia ficaria, Serratia fonticola, Serratia liquefaciens, Serratia marcescens, Shigella dysenteriae, Shigella flexneri, Shigella sonnei, Tatumella ptyseos, Trabulsiella guamensis, Yersinia aleksiciae, Yersinia bercovieri, Yersinia enterocolitica, Yersinia frederiksenii, Yersinia kristensenii, Yersinia pseudotuberculosis, Yersinia rohdei, and Yokenella regensburgei, and

Verrucomicrobiaceae, selected from Prosthecobacter fluviatilis and Akkermansia muciniphila.

In one embodiment, the one or more gut microbiome species is selected from Bacteroides ovatus, Bacteroides plebeius, Bacteroides uniformis, Bacteroidales ph8, Odoribacter splanchnicus, Paraprevotella clara, Paraprevotella unclassified, Alistipes putredinis, Alistipes shahii, Escherichia coli, and Akkermansia muciniphila.

In one embodiment, the one or more gut microbiome species is a human gut microbiome species selected from any of the species described herein.

In one embodiment, the one or more gut microbiome species is sensitive to growth inhibition by an endogenous bile acid, such as CDCA, LCA, and the like. In one embodiment, the one or more gut microbiome species that is sensitive to growth inhibition by a bile acid is a gram positive species, as described herein.

In one embodiment, the pharmaceutical composition comprises a compound of the present application or a pharmaceutically acceptable amino acid conjugate or salt thereof in the amount of 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg. In one embodiment, the pharmaceutical composition comprises a compound of the present application or a pharmaceutically acceptable amino acid conjugate or salt thereof in the amount of 5-25 mg.

In one embodiment, the pharmaceutical composition comprises the one or more gut microbiome species in the amount of 100-10¹² colony forming unit (CFU), 100-10⁹ CFU, 100-10⁶ CFU, 100-10⁵ CFU, 100-10⁴ CFU, or 100-10³ CFU, or 10³-10¹² CFU, 10³-10⁹ CFU, 10³-10⁶ CFU, 10³-10⁵ CFU, or 10³-10⁴ CFU, or 10⁴-10¹² CFU, 10⁴-10⁹ CFU, 10⁴-10⁶ CFU, or 10⁴-10⁵ CFU, or 10⁵-10¹² CFU, 10⁵-10⁹ CFU, or 10⁵-10⁶ CFU, or 10⁶-10¹² CFU, 10⁶-10¹¹ CFU, 10⁶- 10¹⁰ CFU, 10⁶-10⁹ CFU, 10⁶-10⁸ CFU, or 10⁶-10⁷ CFU, or 10⁷-10¹² CFU, 10⁷-10¹¹ CFU, 10⁷-10¹⁰ CFU, 10⁷-10⁹ CFU, or 10⁷-10⁸ CFU, or 10⁸-10¹² CFU, 10⁸-10¹¹ CFU, 10⁸-10¹⁰ CFU, or 10⁸- 10⁹ CFU, or 10⁹-10¹² CFU, 10⁹-10¹¹ CFU, or 10⁹-10¹⁰ CFU.

In one embodiment, a compound of the present application or a pharmaceutically acceptable amino acid conjugate or salt thereof is formulated for oral, parenteral, or topical administration. In one embodiment, a compound of the present application or a pharmaceutically acceptable amino acid conjugate or salt thereof is formulated for oral administration. In one embodiment, a compound of the present application or a pharmaceutically acceptable amino acid conjugate or salt thereof is formulated in a solid form. In one embodiment, a compound of the present application or a pharmaceutically acceptable amino acid conjugate or salt thereof is formulated as a tablet or capsule.

In one embodiment, the one or more gut microbiome species is formulated for oral administration. In one embodiment, the one or more gut microbiome species is formulated as a liquid culture. In one embodiment, the one or more gut microbiome species is formulated as a lyophilized solid (e.g., powder). In one embodiment, the one or more gut microbiome species is formulated as a gel.

In one of the embodiments, the present application relates to a method of using the features of the gut microbiome as biomarkers.

The present application also relates to a method of treating or preventing an FXR mediated disease or condition or a disease or condition in which an abnormal composition of the gut microbiome is involved, comprising administering to a subject in need thereof OCA, or a pharmaceutically acceptable amino acid conjugate or salt thereof, and one or more gut microbiome species. In one embodiment, the present application relates to a method of treating. In one embodiment, the present application relates to a method of preventing. In one embodiment, the present application relates to a method of treating. In one embodiment, the present application relates to a method of preventing.

The present application also relates to OCA, or a pharmaceutically acceptable amino acid conjugate or salt thereof, for use in combination with one or more gut microbiome species in treating or preventing an FXR mediated disease or condition or a disease or condition in which an abnormal composition of the gut microbiome is involved. In one embodiment, the present application relates to treating. In one embodiment, the present application relates to preventing.

The present application also relates to use of OCA, or a pharmaceutically acceptable amino acid conjugate or salt thereof, in the manufacture of a medicament for a combinational therapy with one or more gut microbiome species for the treatment or prevention of an FXR mediated disease or condition or a disease or condition in which an abnormal composition of the gut microbiome is involved. In one embodiment, the present application relates to treatment. In one embodiment, the present application relates to prevention.

The present application also relates to use of OCA, or a pharmaceutically acceptable amino acid conjugate or salt thereof, in combination with one or more gut microbiome species, in treating or preventing an FXR mediated disease or condition or a disease or condition in which an abnormal composition of the gut microbiome is involved. In one embodiment, the present application relates to treating. In one embodiment, the present application relates to preventing.

The present application also relates to a method of enhancing the efficacy of an FXR ligand in treating or preventing a disease or condition, comprising administering to a subject in need thereof one or more gut microbiome species. In one embodiment, the present application relates to a method of treating. In one embodiment, the present application relates to a method of preventing.

The present application also relates to one or more gut microbiome species, for use in enhancing the efficacy of an FXR ligand in treating or preventing a disease or condition. In one embodiment, the present application relates to treating. In one embodiment, the present application relates to preventing.

The present application also relates to use of one or more gut microbiome species in the manufacture of a medicament for enhancing the efficacy of an FXR ligand in the treatment or prevention of a disease or condition. In one embodiment, the present application relates to treatment. In one embodiment, the present application relates to prevention.

The present application also relates to use of one or more gut microbiome species in enhancing the efficacy of an FXR ligand in treating or preventing a disease or condition. In one embodiment, the present application relates to treating. In one embodiment, the present application relates to preventing.

In one embodiment, the one or more gut microbiome species is administered prior to, at the same time as, or following the administration of the FXR ligand. In one embodiment, the one or more gut microbiome species is administered prior to and at the same time as the administration of the FXR ligand. In one embodiment, the one or more gut microbiome species is administered prior to and following the administration of the FXR ligand. In one embodiment, the one or more gut microbiome species is administered at the same time as and following the administration of the FXR ligand. In one embodiment, the one or more gut microbiome species is administered once, twice, three times, or more prior to or following the administration of the FXR ligand. In one embodiment, the one or more gut microbiome species is administered once, twice, three times, or more prior to and at the same time as the administration of the FXR ligand. In one embodiment, the one or more gut microbiome species is administered once, twice, three times, or more prior to and once, twice, three times, or more following the administration of the FXR ligand. In one embodiment, the one or more gut microbiome species is administered at the same time as and once, twice, three times, or more following the administration of the FXR ligand.

In one embodiment, the one or more gut microbiome species is administered once, twice, three times, or more at 10 minutes, 30 minutes, 1 hour, 2 hours, 4 hours, 6 hours, 8 hours, 10 hours, 12 hours, 18 hours, 24 hours, 36 hours, 2 days, 3 days, 4 days, 5 days, 6 days, 7 days, 10 days, 2 weeks, 3 weeks, 4 weeks, or more prior to the administration of the FXR ligand. In one embodiment, the one or more gut microbiome species is administered once, twice, three times, or more at 10 minutes, 30 minutes, 1 hour, 2 hours, 4 hours, 6 hours, 8 hours, 10 hours, 12 hours, 18 hours, 24 hours, 36 hours, 2 days, 3 days, 4 days, 5 days, 6 days, 7 days, 10 days, 2 weeks, 3 weeks, 4 weeks, or more following the administration of the FXR ligand.

In one embodiment, efficacy of an FXR ligand in treating or preventing a disease or condition determined by EC₅₀ value. In one embodiment, efficacy of an FXR ligand in treating or preventing a disease or condition determined by IC₅₀ value. In one embodiment, administration of one or more gut microbiome species as described herein decreases the EC₅₀ value of the FXR ligand in treating or preventing a disease or condition by at least 10%, 20%, 30%, 40%, 50%, 60%, 70%, 80%, 90%, 100%, 150%, 200%, 300%, 400%, 500%, or more. In one embodiment, administration of one or more gut microbiome species as described herein decreases the IC₅₀ value of the FXR ligand in treating or preventing a disease or condition by at least 10%, 20%, 30%, 40%, 50%, 60%, 70%, 80%, 90%, 100%, 150%, 200%, 300%, 400%, 500%, or more.

In one embodiment, the FXR ligand is an endogenous FXR ligand. In one embodiment, the endogenous FXR ligand is as an endogenous FXR agonist. In one embodiment, the endogenous FXR agonist is CDCA, LCA, and the like. In one embodiment, the FXR ligand is an FXR agonist. In one embodiment, the FXR agonist is OCA.

In one embodiment, the disease or condition is an FXR mediated disease or condition. Examples of the FXR mediated diseases or conditions include, but not limited to, liver diseases such as cholestatic liver disease such as primary biliary cirrhosis (PBC), primary sclerosing cholangitis (PSC), portal hypertension, bile acid diarrhea, chronic liver disease, nonalcoholic fatty liver disease (NAFLD), nonalcoholic steatohepatitis (NASH), hepatitis B, hepatitis C, alcoholic liver disease, liver damage due to progressive fibrosis, and liver fibrosis. Examples of FXR mediated diseases also include hyperglycemia, diabetes, obesity, insulin resistance, hyperlipidemia, high LDL-cholesterol, high HDL-cholesterol, high triglycerides, cardiovascular disease, and fibrosis.

NAFLD is a medical condition that is characterized by the buildup of fat (called fatty infiltration) in the liver. NAFLD is one of the most common causes of chronic liver disease, and encompasses a spectrum of conditions associated with lipid deposition in hepatocytes. It ranges from steatosis (simple fatty liver), to nonalcoholic steatohepatitis (NASH), to advanced fibrosis and cirrhosis. The disease is mostly silent and is often discovered through incidentally elevated liver enzyme levels. NAFLD is strongly associated with obesity and insulin resistance and is currently considered by many as the hepatic component of the metabolic syndrome.

Nonalcoholic steatohepatitis (NASH) is a condition that causes inflammation and accumulation of fat and fibrous (scar) tissue in the liver. Liver enzyme levels in the blood may be more elevated than the mild elevations seen with nonalcoholic fatty liver (NAFL). Although similar conditions can occur in people who abuse alcohol, NASH occurs in those who drink little to no alcohol. NASH affects 2 to 5 percent of Americans, and is most frequently seen in people with one of more of the following conditions: obesity, diabetes, hyperlipidemia, insulin resistance, uses of certain medications, and exposure to toxins. NASH is an increasingly common cause of chronic liver disease worldwide and is associated with increased liver-related mortality and hepatocellular carcinoma, even in the absence of cirrhosis. NASH progresses to cirrhosis in 15-20% of affected individuals and is now one of the leading indications for liver transplantation in the United States. At present there are no approved therapies for NASH.

Fibrosis refers to a condition involving the development of excessive fibrous connective tissue, e.g., scar tissue, in a tissue or organ. Such generation of scar tissue may occur in response to infection, inflammation, or injury of the organ due to a disease, trauma, chemical toxicity, and so on. Fibrosis may develop in a variety of different tissues and organs, including the liver, kidney, intestine, lung, heart, etc.

In one embodiment, the fibrosis is selected from the group consisting of liver fibrosis, kidney fibrosis, and intestinal fibrosis.

In one embodiment, the liver fibrosis is associated with a disease selected from the group consisting of hepatitis B; hepatitis C; parasitic liver diseases; post-transplant bacterial, viral and fungal infections; alcoholic liver disease (ALD); non-alcoholic fatty liver disease (NAFLD); non-alcoholic steatohepatitis (NASH); liver diseases induced by methotrexate, isoniazid, oxyphenistatin, methyldopa, chlorpromazine, tolbutamide, or amiodarone; autoimmune hepatitis; sarcoidosis; Wilson's disease; hemochromatosis; Gaucher's disease; types III, IV, VI, IX and X glycogen storage diseases; α₁-antitrypsin deficiency; Zellweger syndrome; tyrosinemia; fructosemia; galactosemia; vascular derangement associated with Budd-Chiari syndrome, veno-occlusive disease, or portal vein thrombosis; and congenital hepatic fibrosis.

In another embodiment, the intestinal fibrosis is associated with a disease selected from the group consisting of Crohn's disease, ulcerative colitis, post-radiation colitis, and microscopic colitis.

In another embodiment, the renal fibrosis is associated with a disease selected from the group consisting of diabetic nephropathy, hypertensive nephrosclerosis, chronic glomerulonephritis, chronic transplant glomerulopathy, chronic interstitial nephritis, and polycystic kidney disease.

Primary biliary cirrhosis (PBC) is an autoimmune disease of the liver marked by the slow progressive destruction of the small bile ducts of the liver, with the intralobular ducts (Canals of Hering) affected early in the disease. When these ducts are damaged, bile builds up in the liver (cholestasis) and over time damages the tissue. This can lead to scarring, fibrosis and cirrhosis. Primary biliary cirrhosis is characterized by interlobular bile duct destruction. Histopathologic findings of primary biliary cirrhosis include: inflammation of the bile ducts, characterized by intraepithelial lymphocytes, and periductal epithelioid granulomata. There are 4 stage of PBC.

Stage 1—Portal Stage: Normal sized triads; portal inflammation, subtle bile duct damage. Granulomas are often detected in this stage.

Stage 2—Periportal Stage: Enlarged triads; periportal fibrosis and/or inflammation. Typically this stage is characterized by the finding of a proliferation of small bile ducts.

Stage 3—Septal Stage: Active and/or passive fibrous septa.

Stage 4—Biliary Cirrhosis: Nodules present; garland

Primary sclerosing cholangitis (PSC) is a disease of the bile ducts that causes inflammation and subsequent obstruction of bile ducts both at a intrahepatic (inside the liver) and extrahepatic (outside the liver) level. The inflammation impedes the flow of bile to the gut, which can ultimately lead to cirrhosis of the liver, liver failure and liver cancer.

As used herein, a “cholestatic condition” refers to any disease or condition in which bile excretion from the liver is impaired or blocked, which can occur either in the liver or in the bile ducts. Intrahepatic cholestasis and extrahepatic cholestasis are the two types of cholestatic conditions. Intrahepatic cholestasis (which occurs inside the liver) is most commonly seen in primary biliary cirrhosis, primary sclerosing cholangitis, sepsis (generalized infection), acute alcoholic hepatitis, drug toxicity, total parenteral nutrition (being fed intravenously), malignancy, cystic fibrosis, and pregnancy. Extrahepatic cholestasis (which occurs outside the liver) can be caused by bile duct tumors, strictures, cysts, diverticula, stone formation in the common bile duct, pancreatitis, pancreatic tumor or pseudocyst, and compression due to a mass or tumor in a nearby organ.

In one embodiment, a cholestatic condition is defined as having an abnormally elevated serum level of alkaline phosphatase, γ-glutamyl transpeptidase (GGT), and/or 5′ nucleotidase. In another embodiment, a cholestatic condition is further defined as presenting with at least one clinical symptom. In one embodiment, the symptom is itching (pruritus). In another embodiment, a cholestatic condition is selected from the group consisting of primary biliary cirrhosis (PBC), primary sclerosing cholangitis (PBS), drug-induced cholestasis, hereditary cholestasis, and intrahepatic cholestasis of pregnancy.

Clinical symptoms and signs of a cholestatic condition include: itching (pruritus), fatigue, jaundiced skin or eyes, inability to digest certain foods, nausea, vomiting, pale stools, dark urine, and right upper quadrant abdominal pain. A patient with a cholestatic condition can be diagnosed and followed clinically based on a set of standard clinical laboratory tests, including measurement of levels of alkaline phosphatase, γ-glutamyl transpeptidase (GGT), 5′ nucleotidase, bilirubin, bile acids, and cholesterol in a patient's blood serum. Generally, a patient is diagnosed as having a cholestatic condition if serum levels of all three of the diagnostic markers alkaline phosphatase, GGT, and 5′ nucleotidase, are considered abnormally elevated. The normal serum level of these markers may vary to some degree from laboratory to laboratory and from procedure to procedure, depending on the testing protocol. Thus, a physician will be able to determine, based on the specific laboratory and test procedure, what an abnormally elevated blood level is for each of the markers. For example, a patient suffering from a cholestatic condition generally has greater than about 125 IU/L alkaline phosphatase, greater than about 65 IU/L GGT, and greater than about 17 NIL 5′ nucleotidase in the blood. Because of the variability in the level of serum markers, a cholestatic condition may be diagnosed on the basis of abnormal levels of these three markers in addition to at least one of the symptoms mentioned above, such as itching (pruritus).

In one embodiment, the subject is not suffering from a cholestatic condition associated with a disease or condition selected from the group consisting of primary liver and biliary cancer, metastatic cancer, sepsis, chronic total parenteral nutrition, cystic fibrosis, and granulomatous liver disease. In embodiments, the fibrosis to be treated or prevented occurs in an organ where FXR is expressed.

In one embodiment, the disease or condition is a disease or condition in which an abnormal composition of the gut microbiome is involved. Examples of the disease or condition in which an abnormal composition of the gut microbiome is involved includes autoimmune diseases, celiac disease, allergic gastroenteropathies, allergies, Type 1 diabetes, thyroiditis, rheumatoid arthritis, neuromyelitis optica, irritable bowel disease, functional bowel disorders, inflammatory bowel disease, Crohn's disease, cardiovascular diseases (e.g., high blood pressure, stroke, peripheral artery disease, congestive heart failure, and coronary artery disease), cancer (e.g., gastric cancer, intestinal cancer, and colorectal cancer), metabolic disorders (e.g., hyperlipidemia, high LDL-cholesterol, high HDL-cholesterol, high triglycerides, hyperglycemia, diabetes, and obesity), microbial infections (e.g., infection associated with the use of antibiotics, C. difficile infection), and antibiotic associated diarrhea.

In one embodiment, a compound of the present application or a pharmaceutically acceptable amino acid conjugate or salt thereof and the one or more gut microbiome species are administered concurrently. For example, a compound of the present application or a pharmaceutically acceptable amino acid conjugate or salt thereof and one or more gut microbiome species are administered together in a single pharmaceutical composition with a pharmaceutical acceptable carrier. In another embodiment, a compound of the present application or a pharmaceutically acceptable amino acid conjugate or salt thereof and the one or more gut microbiome species are administered sequentially. For example, a compound of the present application or a pharmaceutically acceptable amino acid conjugate or salt thereof is administered prior or subsequent to the one or more gut microbiome species.

In one embodiment, the pharmaceutical composition is administered orally, parenterally, or topically. In another embodiment, the pharmaceutical composition is administered orally.

In the methods and uses of the present application the active substances may be administered in single daily doses, or in two, three, four or more identical or different divided doses per day, and they may be administered simultaneously or at different times during the day. Usually, the active substances will be administered simultaneously, more usually in a single combined dosage form.

In one aspect, a compound of the present application or a pharmaceutically acceptable amino acid conjugate or salt thereof and the one or more gut microbiome species are administered at dosages substantially the same as the dosages at which they are administered in the respective monotherapies. In one aspect, a compound of the present application or a pharmaceutically acceptable amino acid conjugate or salt thereof is administered at a dosage which is less than (e.g., less than 90%, less than 80%, less than 70%, less than 60%, less than 50%, less than 40%, less than 30%, less than 20%, or less than 10%) its monotherapy dosage. In one aspect, the one or more gut microbiome species is administered at a dosage which is less than (e.g., less than 90%, less than 80%, less than 70%, less than 60%, less than 50%, less than 40%, less than 30%, less than 20%, or less than 10%) its monotherapy dosage. In one aspect, both a compound of the present application or a pharmaceutically acceptable amino acid conjugate or salt thereof and the one or more gut microbiome species are administered at a dosage which is less than (e.g., less than 90%, less than 80%, less than 70%, less than 60%, less than 50%, less than 40%, less than 30%, less than 20%, or less than 10%) their respective monotherapy dosages.

A pharmaceutical composition of the present application may be in any convenient form for oral administration, such as a tablet, capsule, powder, lozenge, pill, troche, elixir, lyophilized powder, solution, granule, suspension, emulsion, syrup or tincture. Slow-release or delayed-release forms may also be prepared, for example in the form of coated particles, multi-layer tablets, capsules within capsules, tablets within capsules, or microgranules.

Solid forms for oral administration may contain pharmaceutically acceptable binders, sweeteners, disintegrating agents, diluents, flavoring agents, coating agents, preservatives, lubricants and/or time delay agents. Suitable binders include gum acacia, gelatin, corn starch, gum tragacanth, sodium alginate, carboxymethylellulose or polyethylene glycol. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable diluents include lactose, sorbitol, manitol, dextrose, kaolin, cellulose, calcium carbonate, calcium silicate or dicalcium phosphate. Suitable flavoring agents include peppermint oil, oil of wintergreen, cherry, orange or raspberry flavoring. Suitable coating agents include polymers or copolymers or acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulfite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further include dispersing agents and/or suspending agents. Suitable suspending agents include sodium carboxymethylcellulose, methylcellulose, hydroxypropyl methylcellulose, polyvinylpyrrolidone, sodium alginate or cetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Emulsions for oral administration may further include one or more emulsifying agents. Suitable emulsifying agents include dispersing agents as exemplified above or natural gums such as gum acacia or gum tragacanth.

Pharmaceutical compositions of the present application may be prepared by blending, grinding, homogenizing, suspending, dissolving, emulsifying, dispersing and/or mixing a compound of the present application or a pharmaceutically acceptable amino acid conjugate or salt thereof and/or the one or more gut microbiome species, together with the selected excipient(s), carrier(s), adjuvant(s) and/or diluent(s). One type of pharmaceutical composition of the present application in the form of a tablet or capsule may be prepared by (a) preparing a first tablet comprising at least one of the active substances selected from a compound of the present application or a pharmaceutically acceptable amino acid conjugate or salt thereof, together with any desired excipient(s), carrier(s), adjuvant(s) and/or diluent(s), and (b) preparing a second tablet or a capsule, wherein the second tablet or the capsule includes the remaining active substance(s) (i.e., the one or more gut microbiome species) and the first tablet. Another type of pharmaceutical composition of the present application in the form of a capsule may be prepared by (a) preparing a first capsule comprising at least one of the active substances selected from a compound of the present application or a pharmaceutically acceptable amino acid conjugate or salt thereof, together with any desired excipient(s), carrier(s), adjuvant(s) and/or diluent(s), and (b) preparing a second capsule, wherein the second capsule includes the remaining active substance(s) (i.e., the one or more gut microbiome species) and the first capsule. A further type of pharmaceutical composition of the present application in the form of a tablet may be prepared by (a) preparing a capsule comprising at least one of the active substances selected from a compound of the present application or a pharmaceutically acceptable amino acid conjugate or salt thereof, together with any desired excipient(s), carrier(s), adjuvant(s) and/or diluent(s), and (b) preparing a tablet, wherein the tablet includes the remaining active substance(s) (i.e., the one or more gut microbiome species) and the capsule.

In one embodiment, the pharmaceutical compositions of the application is a dosage form which comprises a compound of the present application or a pharmaceutically acceptable amino acid conjugate or salt thereof in an amount of from 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg.

In one embodiment, the pharmaceutical compositions of the application is a dosage form which comprises one or more gut microbiome species in an amount of 100-10¹² CFU, 100-10⁹ CFU, 100-10⁶ CFU, 100-10⁵ CFU, 100-10⁴ CFU, or 100-10³ CFU, or 10³-10¹² CFU, 10³-10⁹ CFU, 10³-10⁶ CFU, 10³-10⁵ CFU, or 10³-10⁴ CFU, or 10⁴-10¹² CFU, 10⁴-10⁹ CFU, 10⁴-10⁶ CFU, or 10⁴-10⁵ CFU, or 10⁵-10¹² CFU, 10⁵-10⁹ CFU, or 10⁵-10⁶ CFU, or 10⁶-10¹² CFU, 10⁶-10¹¹ CFU, 10⁶-10¹⁰ CFU, 10⁶-10⁹ CFU, 10⁶-10⁸ CFU, or 10⁶-10⁷ CFU, or 10⁷-10¹² CFU, 10⁷-10¹¹ CFU, 10⁷-10¹⁰ CFU, 10⁷-10⁹ CFU, or 10⁷-10⁸ CFU, or 10⁸-10¹² CFU, 10⁸-10¹¹ CFU, 10⁸-10¹⁰ CFU, or 10⁸-10⁹ CFU, or 10⁹-10¹² CFU, 10⁹-10¹¹ CFU, or 10⁹-10¹⁰ CFU.

As used herein, the term “obeticholic acid” or “OCA” refers to a compound having the chemical structure:

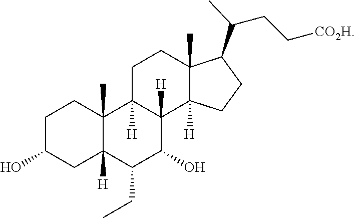

Obeticholic acid is also referred to as obeticholic acid Form 1, INT-747, 3α,7α-dihydroxy-6α-ethyl-5β-cholan-24-oic acid, 6α-ethyl-chenodeoxycholic acid, 6-ethyl-CDCA, 6ECDCA, cholan-24-oic acid, 6-ethyl-3,7-dihydroxy-,(3α,5β, 6α,7α), and can be prepared by the methods described in U.S. Publication No. 2009/0062526 A1, U.S. Pat. No. 7,138,390, and WO2006/122977. The CAS registry number for obeticholic acid is 459789-99-2.

As used herein, the term “amino acid conjugates” refers to conjugates of a compound of the present application with any suitable amino acid. For example, such a suitable amino acid conjugate of a compound of the present application will have the added advantage of enhanced integrity in bile or intestinal fluids. Suitable amino acids include but are not limited to glycine and taurine. Thus, the present application encompasses the glycine and taurine conjugates of OCA. Other conjugates include sarcosine.

As defined herein, the term “metabolite” refers to glucuronidated and sulphated derivatives of the compounds described herein, wherein one or more glucuronic acid or sulphate moieties are linked to compound of the invention. Glucuronic acid moieties may be linked to the compounds through glycosidic bonds with the hydroxyl groups of the compounds (e.g., 3-hydroxyl, 7-hydroxyl, 11-hydroxyl, and/or the hydroxyl of the R₇ group). Sulphated derivatives of the compounds may be formed through sulphation of the hydroxyl groups (e.g., 3-hydroxyl, 7-hydroxyl, 11-hydroxyl, and/or the hydroxyl of the R₇ group). Examples of metabolites include, but are not limited to, 3-O-glucuronide, 7-O-glucuronide, 11-O-glucuronide, 3-O-7-O-diglucuronide, 3-O-11-O-triglucuronide, 7-O-11-O-triglucuronide, and 3-O-7-O-11-O-triglucuronide, of the compounds described herein, and 3-sulphate, 7-sulphate, 11-sulphate, 3,7-bisulphate, 3,11-bisulphate, 7,11-bisulphate, and 3,7,11-trisulphate, of the compounds described herein.

It is to be understood that the isomers arising from asymmetric carbon atoms (e.g., all enantiomers and diastereomers) are included within the scope of the application, unless indicated otherwise. Such isomers can be obtained in substantially pure form by classical separation techniques and by stereochemically controlled synthesis.

“Metagenomics” refers to the study of genetic material recovered directly from environmental samples. Applied in the study of the gut microbiota, it allows comprehensive examination of microbial communities without the need for cultivation. Instead of examining the genomes of individual bacterial strains that have been grown in the laboratory and then trying to reassemble the community of microbes, the metagenomic approach allows analysis of genetic material harvested directly from microbial communities without the need to culture the microbes.

“Shotgun metagenomics” refers to the study of metagenomics through shotgun sequencing.

“Shotgun sequencing” refers to a method used for sequencing DNA by breaking up DNA randomly into numerous small segments and sequencing with chain termination method to obtain reads. Multiple overlapping reads for the target DNA obtained by performing several rounds of fragmentation and sequencing are used to assemble a continuous DNA sequence through analysis of the overlapping ends of different reads.

An “abnormal composition” of the gut microbiome refers to a composition of the gut microbiome where the amount of one or more gut microbiome species is different from the average amount of the one or more species under normal conditions (i.e., when the gut microbiome is not disturbed). In one embodiment, the amount is at least 10%, at least 20%, at least 30%, at least 40%, at least 50%, at least 60%, at least 70%, at least 80%, at least 90%, at least 100%, at least 200%, at least 300%, or at least 500% more or less than the amount under normal conditions.

“Treating”, includes any effect, e.g., lessening, reducing, modulating, or eliminating, that results in the improvement of the condition, disease, disorder, etc. “Treating” or “treatment” of a disease state includes: inhibiting the disease state, i.e., arresting the development of the disease state or its clinical symptoms, or relieving the disease state, i.e., causing temporary or permanent regression of the disease state or its clinical symptoms.

“Preventing” the disease state includes causing the clinical symptoms of the disease state not to develop in a subject that may be exposed to or predisposed to the disease state, but does not yet experience or display symptoms of the disease state.

The term “inhibiting” or “inhibition,” as used herein, refers to any detectable positive effect on the development or progression of a disease or condition. Such a positive effect may include the delay or prevention of the onset of at least one symptom or sign of the disease or condition, alleviation or reversal of the symptom(s) or sign(s), and slowing or prevention of the further worsening of the symptom(s) or sign(s).

“Disease state” means any disease, disorder, condition, symptom, or indication.

The term “effective amount” or “therapeutically effective amount” as used herein refers to an amount of a compound of the present application or a pharmaceutically acceptable amino acid conjugate or salt thereof and the one or more gut microbiome species that produces an acute or chronic therapeutic effect upon appropriate dose administration, alone or in combination. In one embodiment, an effective amount or therapeutically effective amount of a compound of the present application or a pharmaceutically acceptable amino acid conjugate or salt thereof produces an acute or chronic therapeutic effect upon appropriate dose administration in combination with one or more gut microbiome species. The effect includes the prevention, correction, inhibition, or reversal of the symptoms, signs and underlying pathology of a disease/condition (e.g., fibrosis of the liver, kidney, or intestine) and related complications to any detectable extent. An “effective amount” or “therapeutically effective amount” will vary depending on the compound of the present application or a pharmaceutically acceptable amino acid conjugate or salt thereof, the one or more gut microbiome species, the disease and its severity, and the age, weight, etc., of the subject to be treated.

“Pharmacological effect” as used herein encompasses effects produced in the subject that achieve the intended purpose of a therapy. In one embodiment, a pharmacological effect means that primary indications of the subject being treated are prevented, alleviated, or reduced. For example, a pharmacological effect would be one that results in the prevention, alleviation or reduction of primary indications in a treated subject. In another embodiment, a pharmacological effect means that disorders or symptoms of the primary indications of the subject being treated are prevented, alleviated, or reduced. For example, a pharmacological effect would be one that results in the prevention, alleviation or reduction of the disorders or symptoms in a treated subject.

A “pharmaceutical composition” is a formulation containing therapeutic agents such as a compound of the present application or a pharmaceutically acceptable amino acid conjugate or salt thereof and the one or more gut microbiome species, in a form suitable for administration to a subject. In one embodiment, the pharmaceutical composition is in bulk or in unit dosage form. It can be advantageous to formulate compositions in dosage unit form for ease of administration and uniformity of dosage. Dosage unit form as used herein refers to physically discrete units suited as unitary dosages for the subject to be treated; each unit containing a predetermined quantity of active reagent calculated to produce the desired therapeutic effect in association with the required pharmaceutical carrier. The specification for the dosage unit forms of the application are dictated by and directly dependent on the unique characteristics of the active agents and the particular therapeutic effect to be achieved, and the limitations inherent in the art of compounding such an active agent for the treatment of individuals.

The term “unit dosage form” refers to physically discrete units suitable as unitary dosages for humans and other mammals, each unit containing a predetermined quantity of active material calculated to produce the desired therapeutic effect, in association with a suitable pharmaceutical excipient as described herein.

The unit dosage form is any of a variety of forms, including, for example, a capsule, an IV bag, a tablet, a single pump on an aerosol inhaler, or a vial. The quantity of a compound of the present application or a pharmaceutically acceptable amino acid conjugate or salt thereof and/or the one or more gut microbiome species in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this application include powders, sprays, ointments, pastes, creams, lotions, gels, solutions, patches and inhalants. In one embodiment, a compound of the present application or a pharmaceutically acceptable amino acid conjugate or salt thereof and the one or more gut microbiome species are mixed with a pharmaceutically acceptable carrier, and with any preservatives, buffers, or propellants that are required.

The term “flash dose” refers to formulations that are rapidly dispersing dosage forms.

The term “immediate release” is defined as a release of a therapeutic agent (such as a compound of the present application or a pharmaceutically acceptable amino acid conjugate or salt thereof and the one or more gut microbiome species) from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a therapeutic agent from a dosage form over a prolonged period.

A “subject” includes mammals, e.g., humans, companion animals (e.g., dogs, cats, birds, and the like), farm animals (e.g., cows, sheep, pigs, horses, fowl, and the like), and laboratory animals (e.g., rats, mice, guinea pigs, birds, and the like). In one embodiment, the subject is human. In one aspect, the subject is female. In one aspect, the subject is male.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier or excipient” means a carrier or excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

While it is possible to administer a compound of the present application or a pharmaceutically acceptable amino acid conjugate or salt thereof and/or the one or more microbiome species directly without any formulation, a compound of the present application or a pharmaceutically acceptable amino acid conjugate or salt thereof and/or the one or more microbiome species may be administered in the form of a pharmaceutical formulation comprising a pharmaceutically acceptable excipient. This formulation can be administered by a variety of routes including oral, buccal, rectal, intranasal, transdermal, subcutaneous, intravenous, intramuscular, and intranasal.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The application having now been described by way of written description, those of skill in the art will recognize that the application can be practiced in a variety of embodiments and that the foregoing description and examples below are for purposes of illustration and not limitation of the claims that follow.

In the specification, the singular forms also include the plural, unless the context clearly dictates otherwise. Unless defined otherwise, all technical and scientific terms used herein have the same meaning as commonly understood by one of ordinary skill in the art to which this application belongs. In the case of conflict, the present specification will control.

All percentages and ratios used herein, unless otherwise indicated, are by weight.

### EXAMPLES

The application is further illustrated by the following examples, which are not to be construed as limiting this application in scope or spirit to the specific procedures herein described. It is to be understood that the examples are provided to illustrate certain embodiments and that no limitation to the scope of the application is intended. It is to be further understood that resort may be had to various other embodiments, modifications, and equivalents thereof which may suggest themselves to those skilled in the art without departing from the spirit of the present application and/or scope of the appended claims.

### Example 1: Material and Methods

### Sample Collection

Twenty-four eligible subjects were enrolled and randomized to 1 of 3 treatment groups (5 mg, 10 mg, or 25 mg) in a treatment ratio of 1:1:1. The study comprised single dose and multiple dose phases. The randomized dose administered in the single dose phase was the subject's dose level for the multiple dose phase. A single dose of OCA (5 mg, 10 mg, or 25 mg) was administered on Day 1. On Day 4, the multiple dose phase began at the same dose level (5 mg, 10 mg, or 25 mg), with subjects receiving OCA once daily for 14 days. The last dose was given to subjects on Day 17. Subjects remained at the inpatient trial site from Day 0 until the morning of Day 30, and returned to the study site for collection of a sample on Days 35, 37, 39, and 44.

Stool specimens for microbiota genome testing were collected up to 2 days prior to submitting them on Day 0, on Day 15, 16, or 17 prior to submitting them on the same day, and up to 2 days prior to submitting them on Day 37. One Day 1, pre-dose blood samples were collected, and serial blood samples were obtained from Day 1 to Day 3 following administration on Day 1 (see Table 1). Multiple dose phase started on Day 4 and lasted through Day 17, during which subjects received once daily doses of OCA (5 mg, 10 mg, or 25 mg) and pre-dose blood samples were drawn (see Table 1). After Day 17, blood samples were collected as shown in Table 1 until Day 30.

FGF19 analysis was done in the 5 and 10 mg dose group according to time points shown in columns 1 (study day) and 2 (collection time); the numbers of subjects in each dose group are included in parenthesis in columns 3. Metagenomics analysis was done on 5, 10, and 25 mg dose groups on stool samples collected on three time points discussed above.

TABLE 1

| | | FGF19 | Metagenomics | |
|---|---|---|---|---|
| | | OCA dose = | OCA dose = | |
| day | hour | 5,10 | 5,10,25 | Session |
| 1 | predose | (8,8) | (8,8,8) | Day 0~3 |
| 1 | 1 | (8,8) | | Day 0~3 |
| 1 | 2 | (8,8) | | Day 0~3 |
| 1 | 4 | (8,8) | | Day 0~3 |
| 1 | 6 | (8,8) | | Day 0~3 |
| 1 | 8 | (8,8) | | Day 0~3 |
| 1 | 10 | (8,8) | | Day 0~3 |
| 1 | 12 | (8,8) | | Day 0~3 |
| 1 | 14 | (8,8) | | Day 0~3 |
| 2 | 24 | (8,8) | | Day 0~3 |
| 3 | 48 | (8,8) | | Day 0~3 |
| 4 | predose | (8,8) | | Day 4~16 |
| 7 | predose | (7,8) | | Day 4~16 |
| 12 | predose | (7,8) | | Day 4~16 |
| 14 | predose | (7,8) | | Day 4~16 |
| 15 | predose | (7,8) | (7,7,7) | Day 4~16 |
| 17 | predose | (7,8) | | Day 17~30 |
| 17 | 1 | (7,8) | | Day 17~30 |
| 17 | 2 | (7,8) | | Day 17~30 |
| 17 | 4 | (7,8) | | Day 17~30 |
| 17 | 6 | (7,8) | | Day 17~30 |
| 17 | 8 | (7,8) | | Day 17~30 |
| 17 | 10 | (7,8) | | Day 17~30 |
| 17 | 12 | (7,8) | | Day 17~30 |
| 17 | 14 | (7,8) | | Day 17~30 |
| 18 | 24 | (7,8) | | Day 17~30 |
| 19 | 48 | (7,8) | | Day 17~30 |
| 20 | 72 | (7,8) | | Day 17~30 |
| 21 | 96 | (7,8) | | Day 17~30 |
| 22 | 120 | (7,8) | | Day 17~30 |
| 23 | 144 | (7,8) | | Day 17~30 |
| 24 | 168 | (7,8) | | Day 17~30 |
| 25 | 192 | (7,8) | | Day 17~30 |
| 26 | 216 | (7,8) | | Day 17~30 |
| 27 | 240 | (7,8) | | Day 17~30 |
| 28 | 264 | (7,8) | | Day 17~30 |
| 29 | 288 | (7,8) | | Day 17~30 |
| 30 | 312 | (7,8) | | Day 17~30 |
| 44 | 648 | (7,6) | (7,8,7) | Day after 30 |

For microbiome analysis, stool samples were collected on day 0, day 15 or 16, and day 37. FGF19 and C4

To study the relationship of FGF19 and microbiome, FGF19 level was measured on 5 mg and 10 mg OCA dose group hourly on day 1 and day 17, daily from day 4 to day 7, and daily from day 18 to day 30, and day 44. The day 1 pre-dose FGF19 level was used as a match of day 1 microbiome measure. The average of FGF19 value from day 4 to day 17 predose was calculated as a match of day 15 or day 16 microbiome measure. The average level of FGF19 on day 30 and day 44 was used as a match of microbiome on day 37. The average of multiple time points was taken to match the three time points of microbiome measurements.

C4 level was measured on 5 mg and 10 mg OCA dose groups. The day 1 pre-dose C4 was used as a match of day 1 microbiome measurement. The average level of C4 from day 4 to day 17 pre-dose was calculated as a match of day 15 or 16 microbiome measurement. The average C4 level on day 30 and day 44 was used as a match of microbiome on day 37. The average of multiple time points was taken to match the three time points of microbiome measurements.

### Bacterial Composition

The bacterial abundance on species level was generated from MetaPhlAn2 (Segata et al., Nat. Methods 9, 811 (2012)). 341 species were identified from the dataset.

### Metagenomic Sequencing

The adaptors, human reads contamination, and low quality sequences were removed from the raw sequencing data using software kneaddata. Trimmomatic (Bolger et al., Bioinformatics 30, 2114 (2014)) was invoked by kneaddata for removing adaptor sequence, trimming low quality bases, and removing low quality reads. Bowtie (Langmead and Salzberg, Nat. Methods 9, 357 (2012)) was invoked by kneaddata for human reads detection.
Removing sequencing noise: For each sample, if the abundance of one species was smaller than 0.05% of the maximum abundance of all species in the sample, it was considered as noise, and the abundance of this species in the sample was set as 0.
Imputing missing value (i.e. Os): Because log 2 transformation was needed in further analysis, adjustment factor (e) was added to the abundance matrix, where e equals 50% of the minimum non-zero abundance value of all samples.
Removing low abundance species: A species was removed if the relative abundance was lower than 0.1% in >=95% of samples. 128 species was kept for further analysis.

### Functional Profile

HUMAnN2 (Abubucker et al., PLoS Comput. Biol. 8, e1002358 (2012)) was used to calculate gene and pathway abundance from metagenomic sequencing data. Uniref50 (UniProt® Consortium, Nucleic Acids Res. 43, D204 (2015)) was used for gene family definition. MetaCyc (Caspi et al., Nucleic Acids Res. 42, D459 (2014)) and KEGG (Kanehisa and Goto, Nucleic Acids Res. 28, 27 (2000); Kanehisa et al., Nucleic Acids Res. 44, D457 (2016)) were used for pathway analysis.
Gene family identification: Initially, 989251 gene families were identified from the dataset by HUMAnN2. Quantile normalization was implemented. For each sample, the RPK values were added up till 90% percentile of all genes (normalization factor, nf). RPK value of each gene in each sample was then divided by the nf of the sample, and multiplied with the mean of nf of all samples. 683270 uncharacterized genes were removed. Genes undetected in over 90% of samples (101994) were removed. 203987 genes were kept. Due to the large number of genes identified, 25% of the most varying genes were selected for further analysis. The most varying genes were defined by the coefficient variation (cv=sd/mean) of log 2 transformed value.
MetaCyc pathway identification: HUMAnN2 identified 567 MetaCyc pathways from the dataset. Quantile normalization was then performed. For each sample, the abundance values were added up till 90% percentile of all pathways (normalization factor, nf). Each pathway abundance in each sample was divided by the nf of the sample, and then multiplied with the mean of nf for all samples. The low abundance pathways (52), which were not detected on over 90% of all samples, were removed. 515 high quality MetaCyc pathways were kept for further analysis.
KEGG pathway identification: HUMAnN2 identified 160 KEGG pathways from the dataset. Quantile normalization was applied, and low abundance pathways were removed. 66 high quality KEGG pathways were kept for further analysis.

### Statistical Analysis

Species differential abundance analysis: The time effect of OCA treatment was studied on three OCA dose groups separately using Friedman rank test. The dose effect of OCA treatment was studied on three time point separately, and Kruskal-Wallis test was used.
Gene differential abundance analysis: For each of the most varying genes, repeated-measure ANOVA was performed to study the time effect and OCA dose effect on gene abundance change.

gene abundance˜aov(day*OCAdose+error(subject ID))
Pathway differential abundance analysis: For each of the identified pathways, repeated measure ANOVA was also performed to study the time effect and OCA dose effect on pathway abundance change.

pathway abundance˜aov(day*OCAdose+error(subject ID))
FGF19 analysis: The GEE model was applied in order to study the relationship of FGF19 change with the microbiome, gene family, and the pathway abundance change over time. The R function geeglm from R package geepack was used for gee modeling. Both FGF19 level and species/gene/pathway relative abundance were log 2 transformed before applying GEE model.

Geeglm(y·log 2˜fgf19·log 2, id=sID, corstr=“exchangeable”)
The dose and time(hour) effect of OCA treatment on FGF19 change was tested using the GEE model.

Geeglm(fgf19·log 2˜hour+dose+hour:dose, id=sID, corstr=“exchangeable”)
C4 analysis: The GEE model was applied in association study of C4 and microbiome species. Both C4 and species relative abundance were log 2 transformed before applying GEE model.

Geeglm(species·log 2˜C4·log 2, id=sID, corstr=“exchangeable”)

### Example 2: Time Effect of OCA Treatment on Gut Microbiome Species

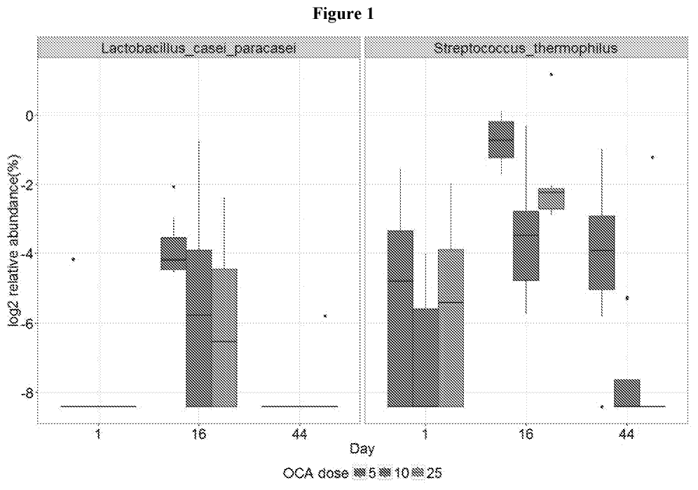

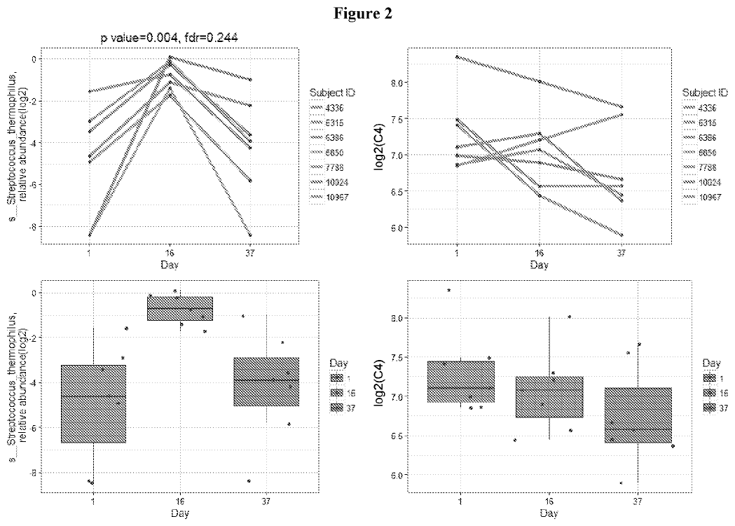

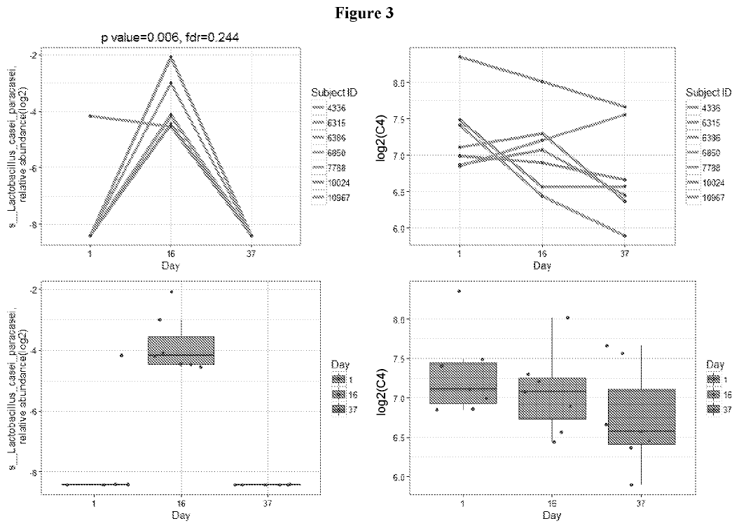

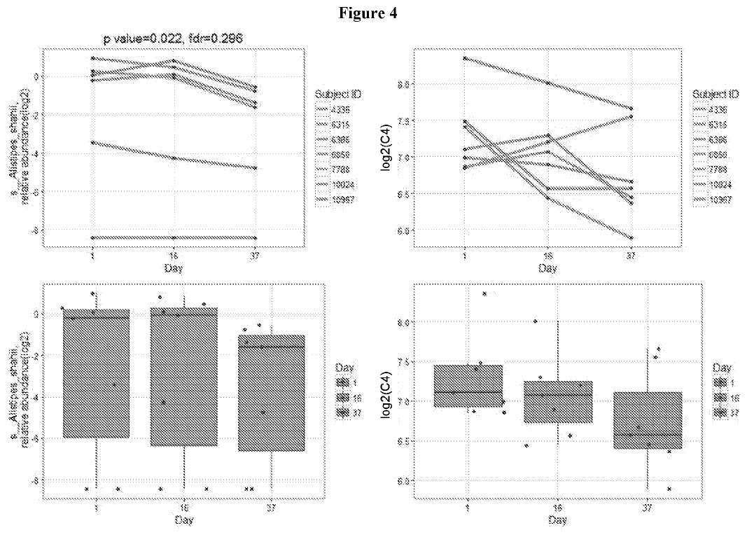

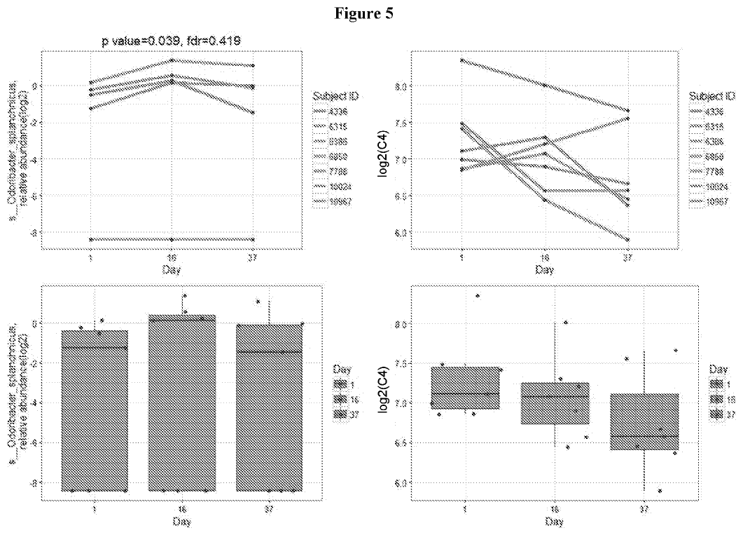

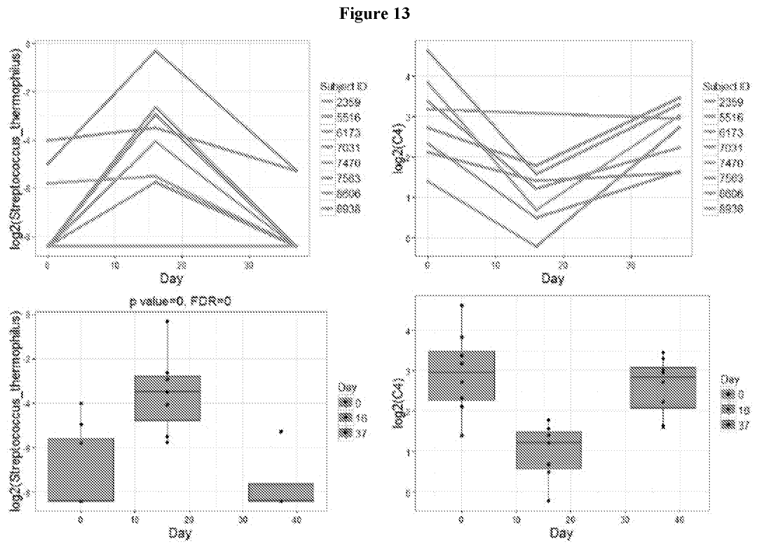

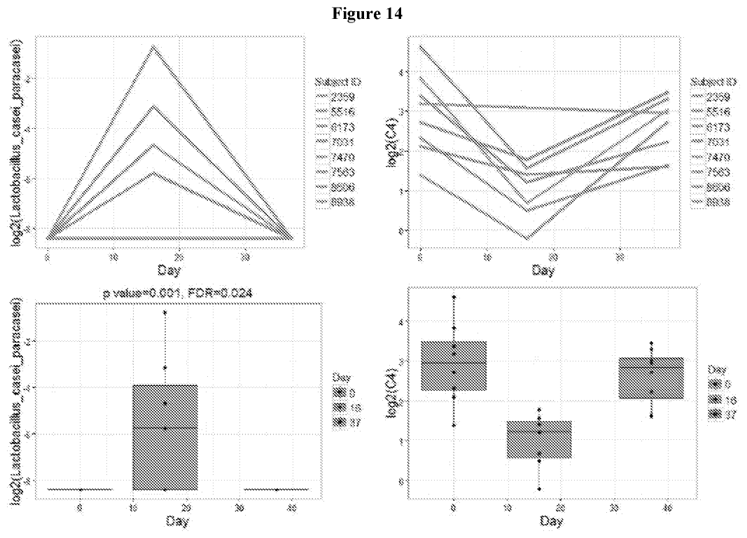

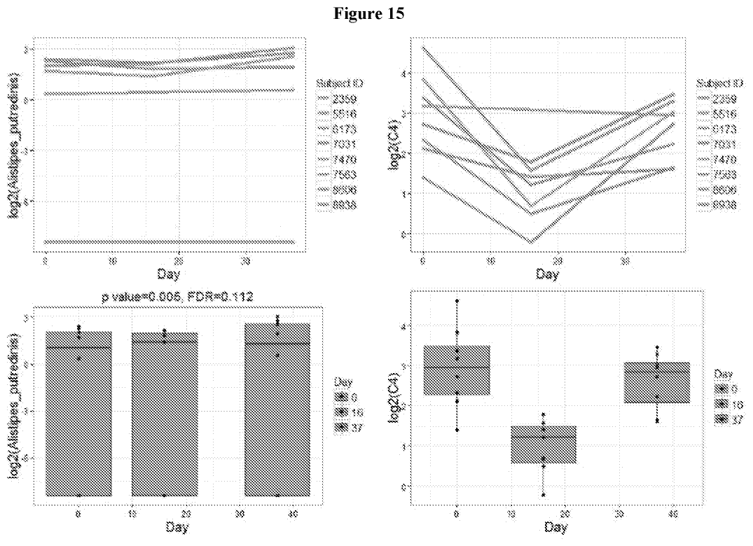

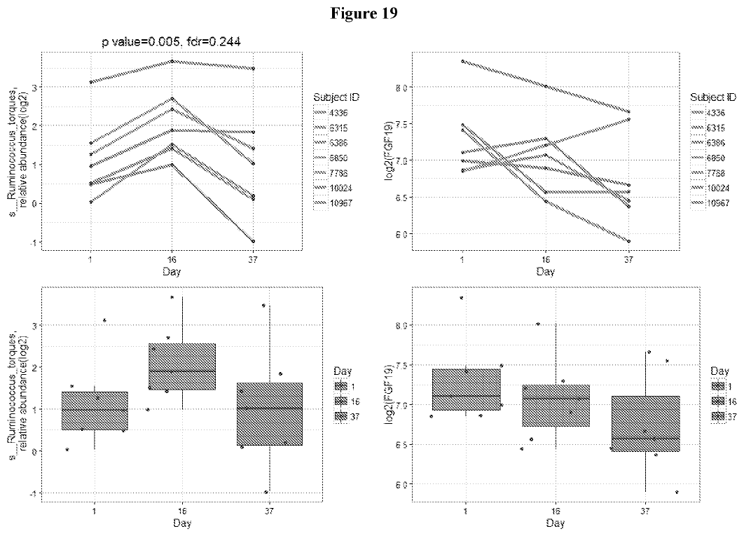

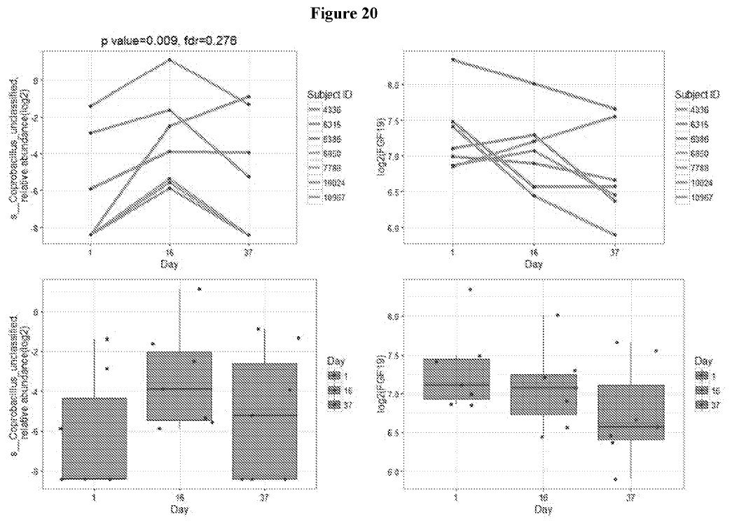

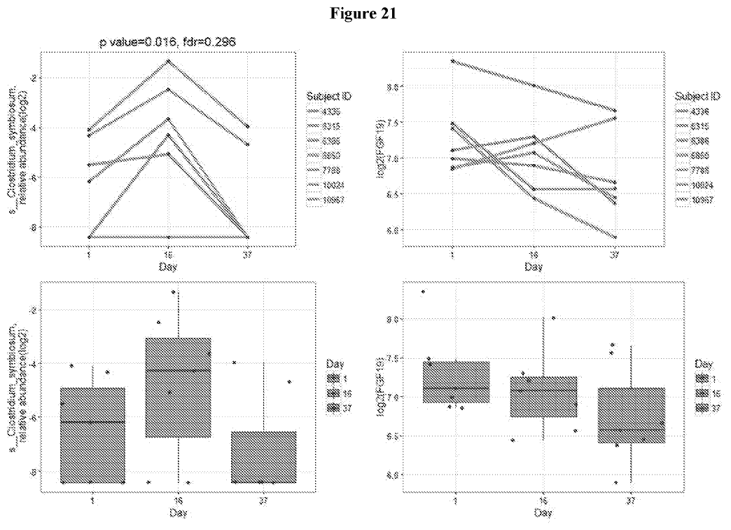

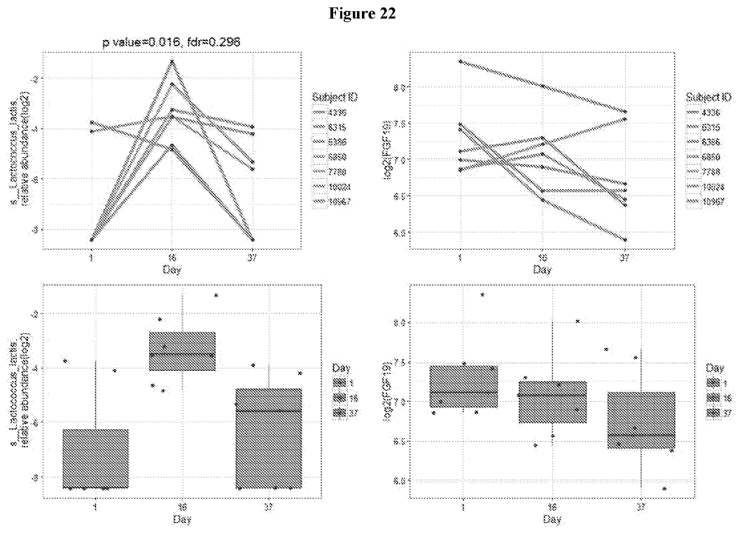

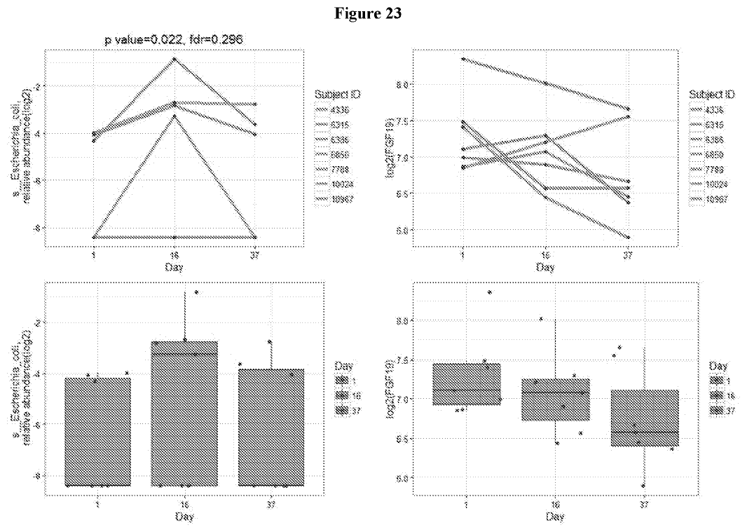

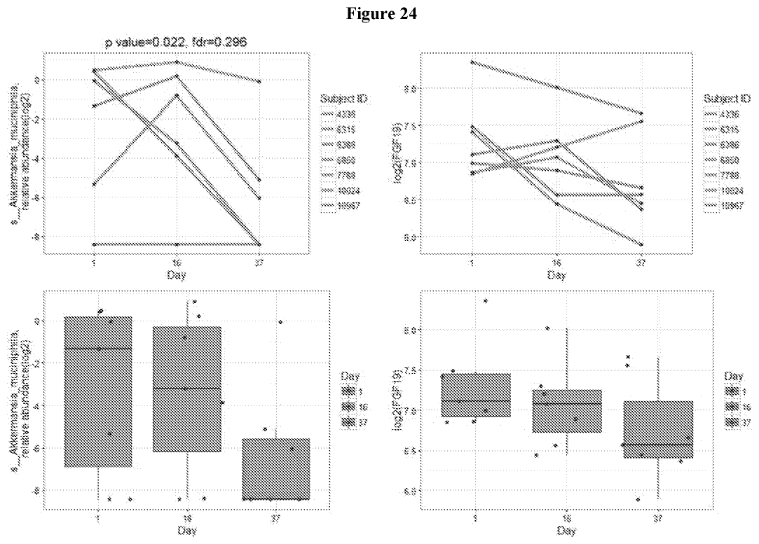

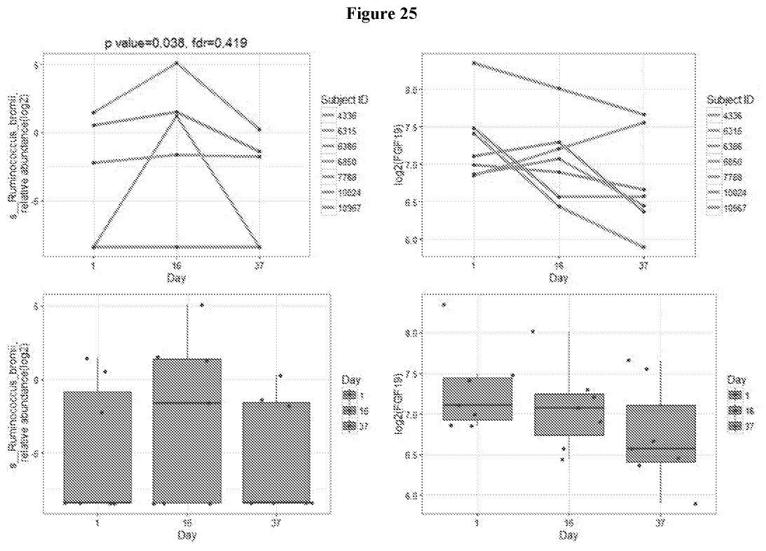

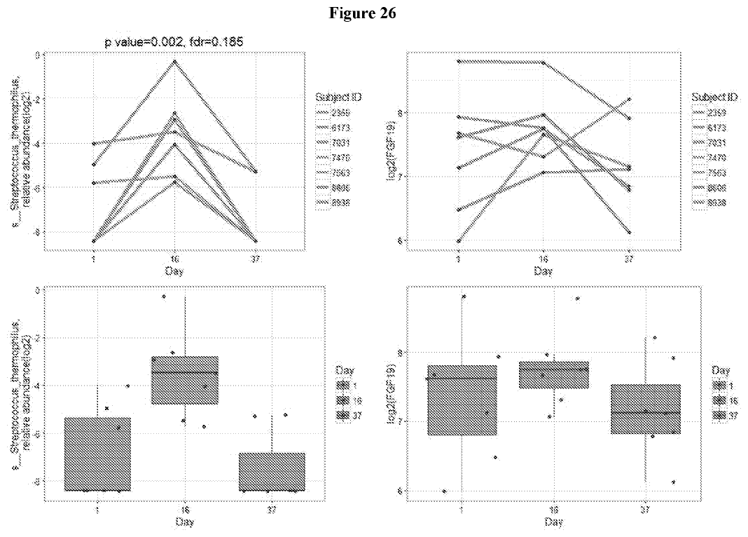

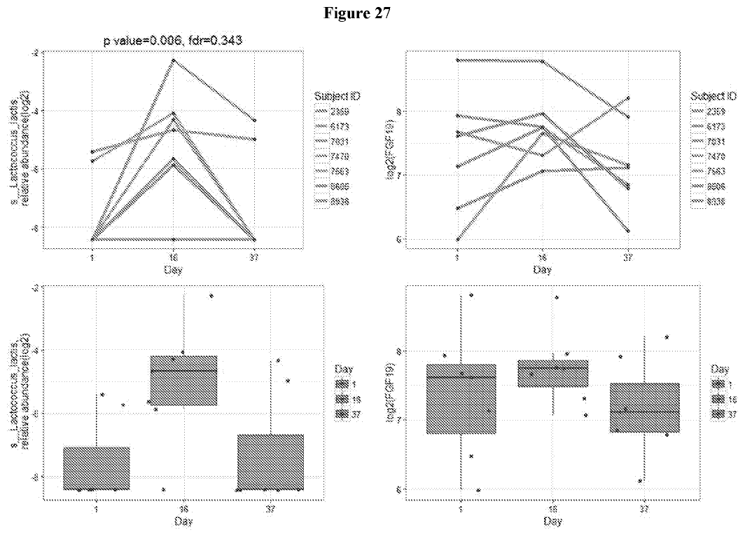

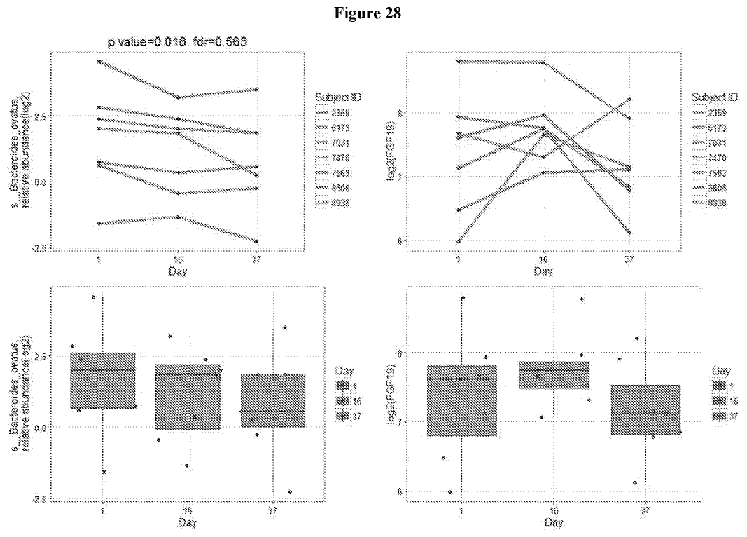

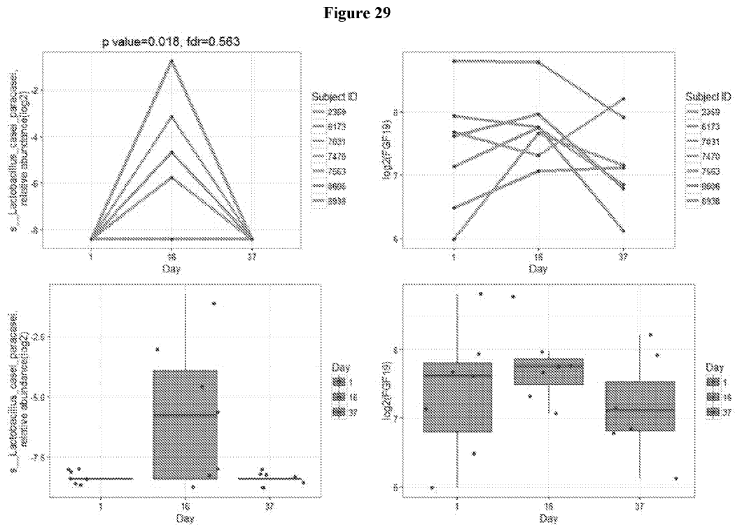

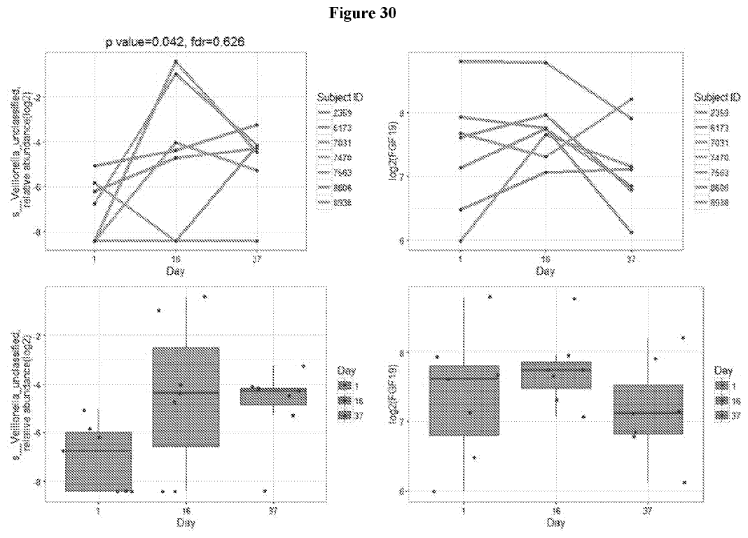

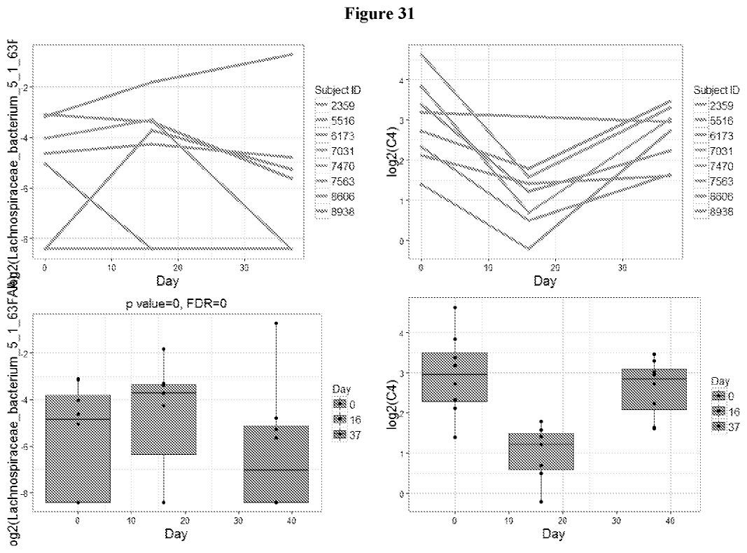

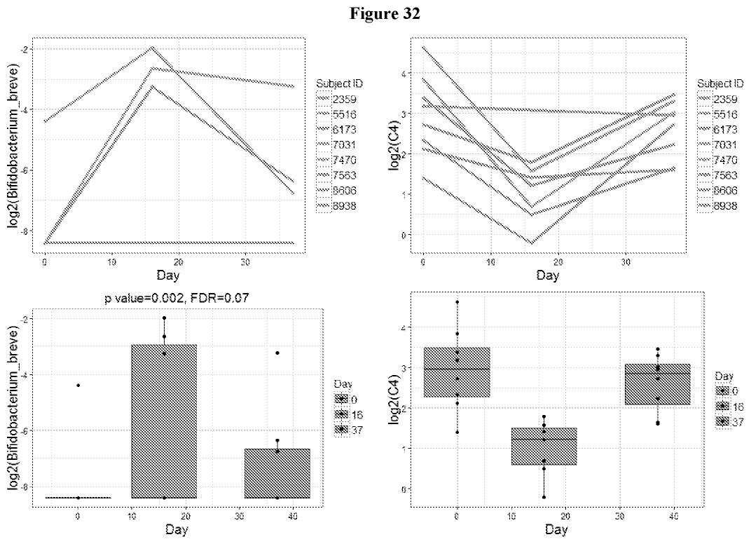

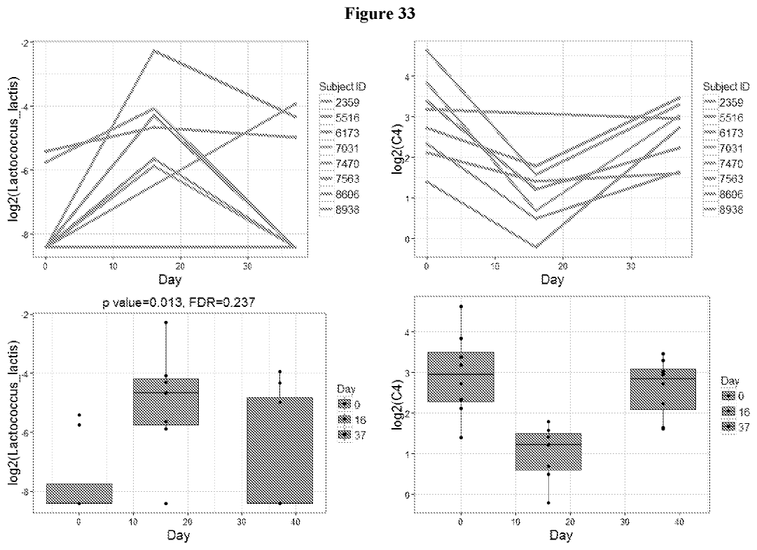

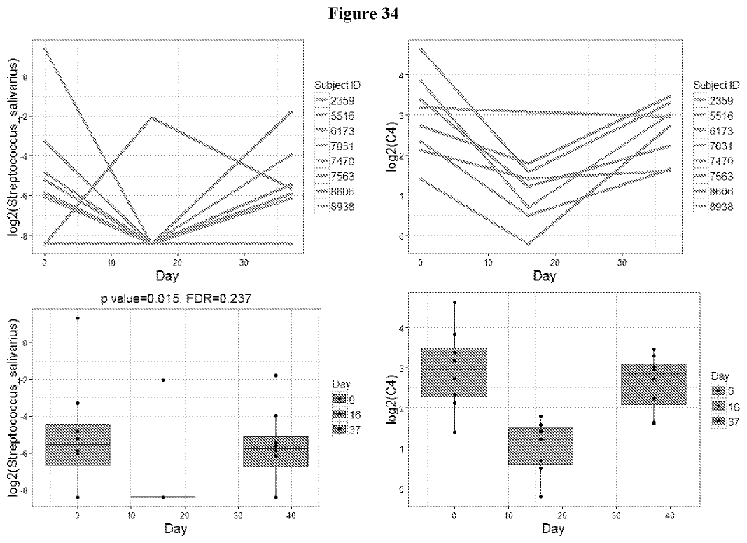

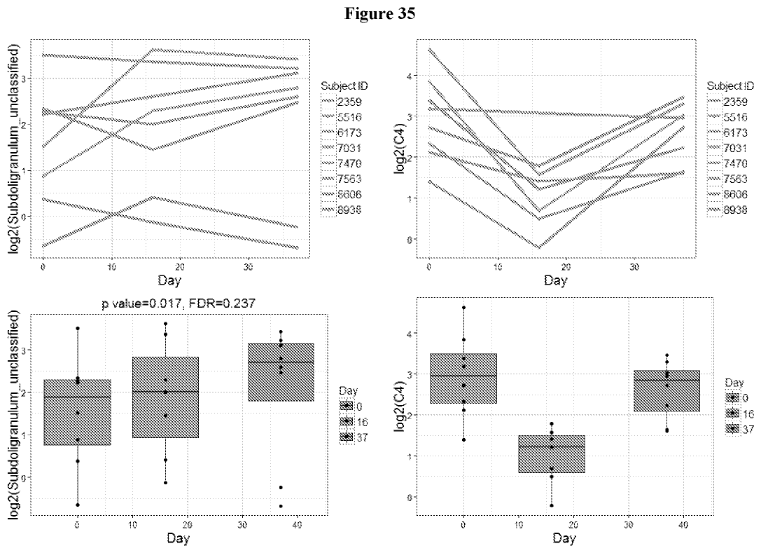

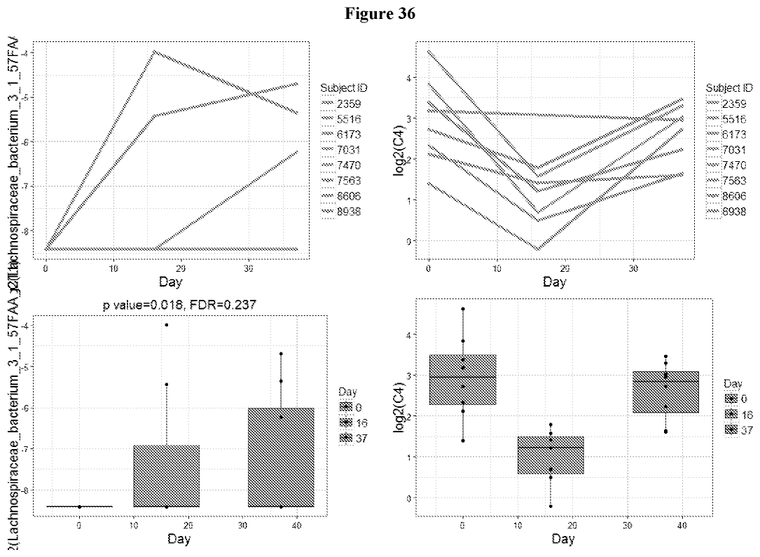

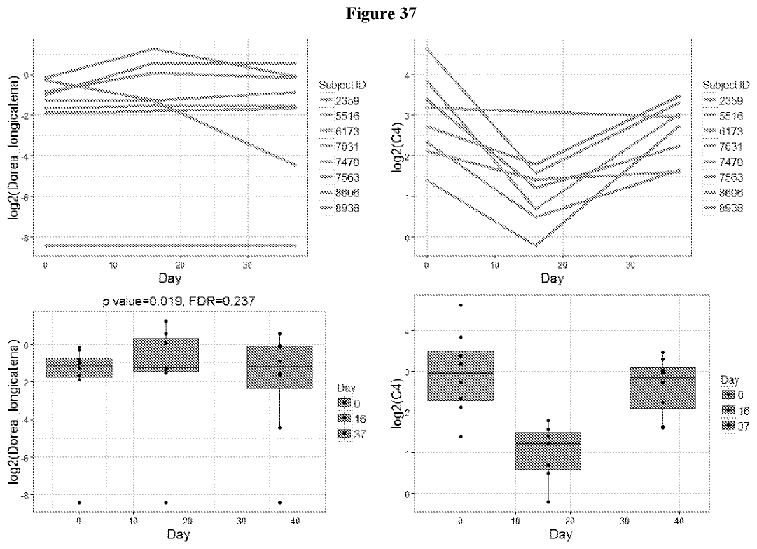

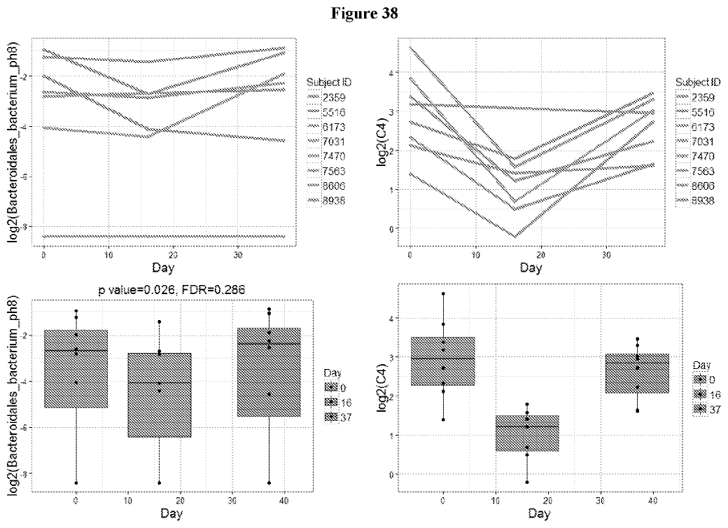

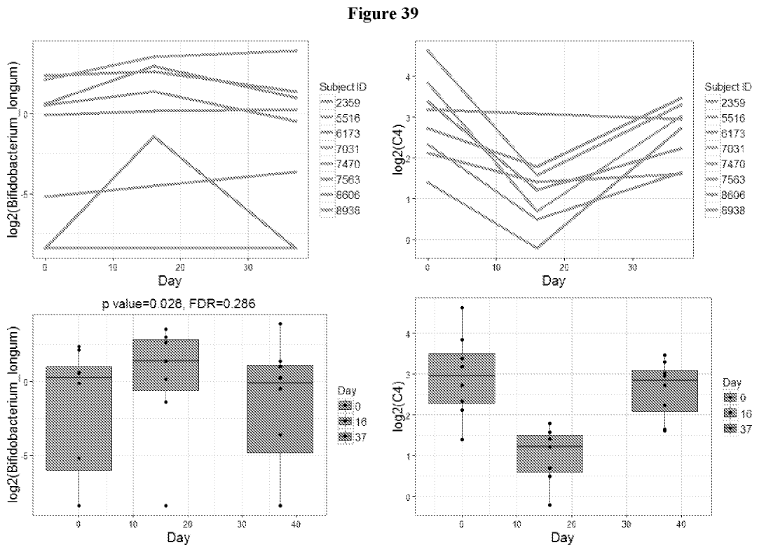

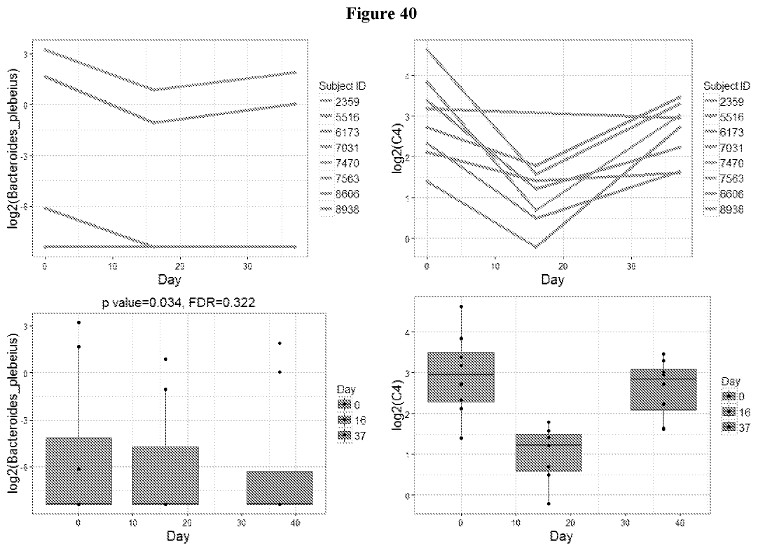

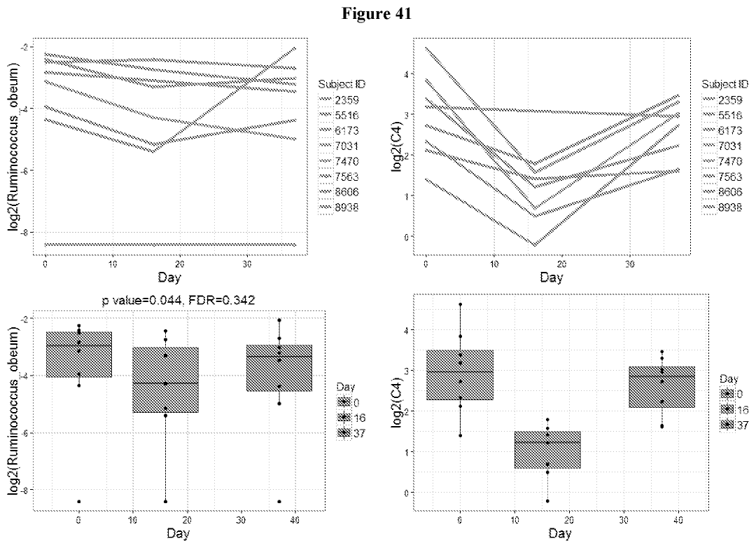

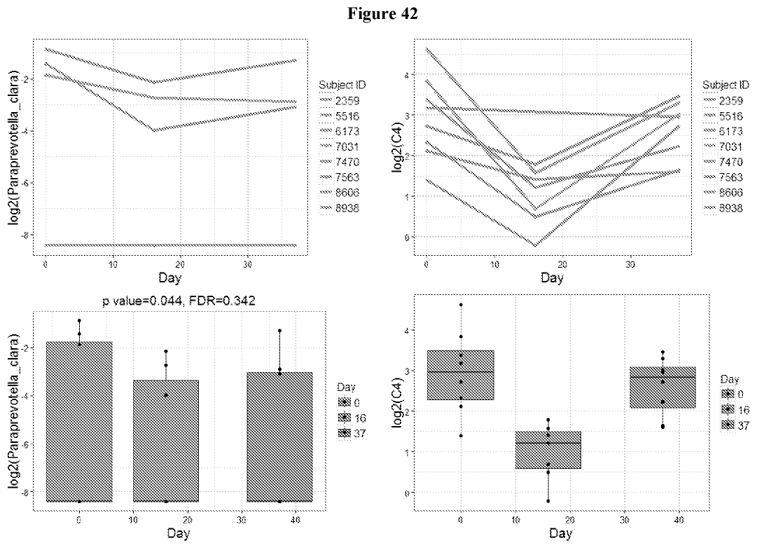

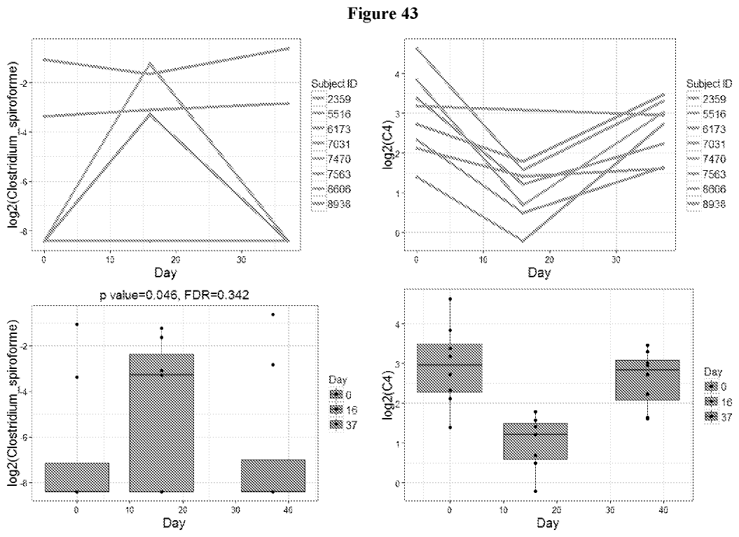

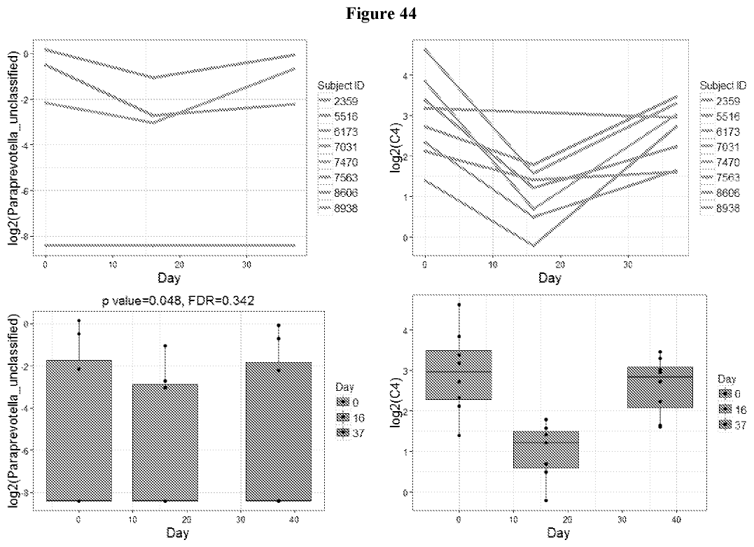

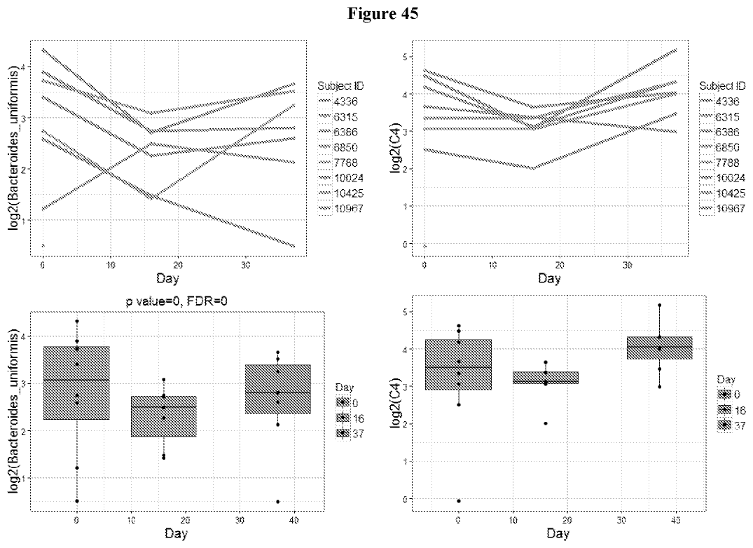

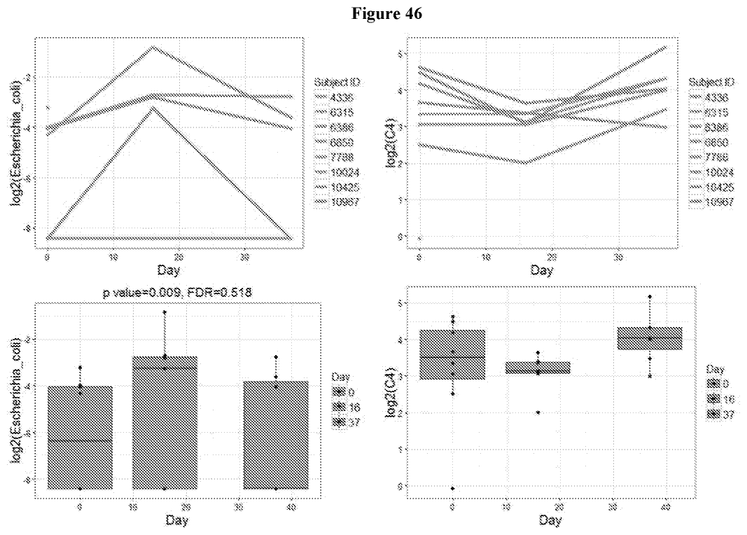

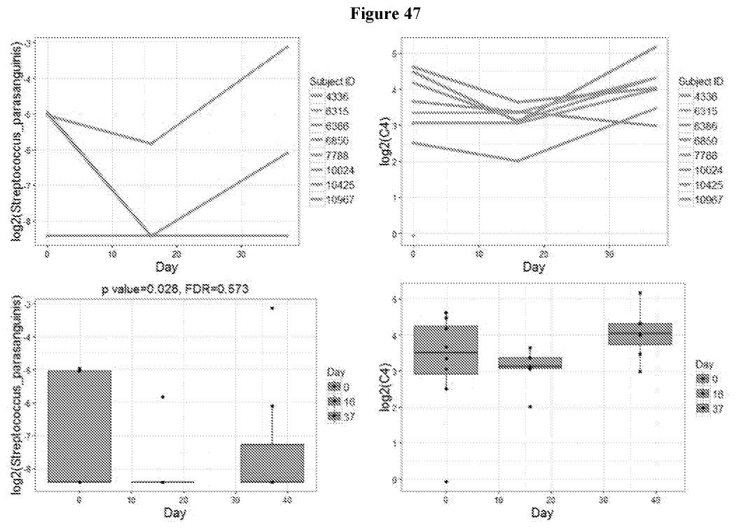

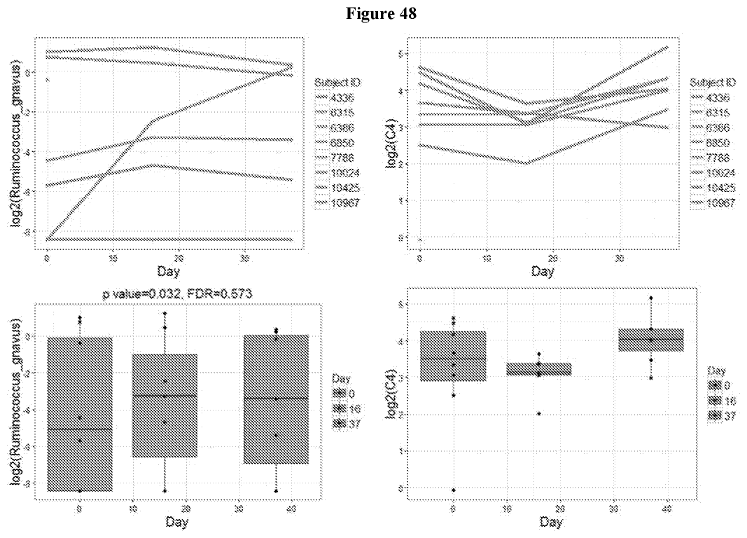

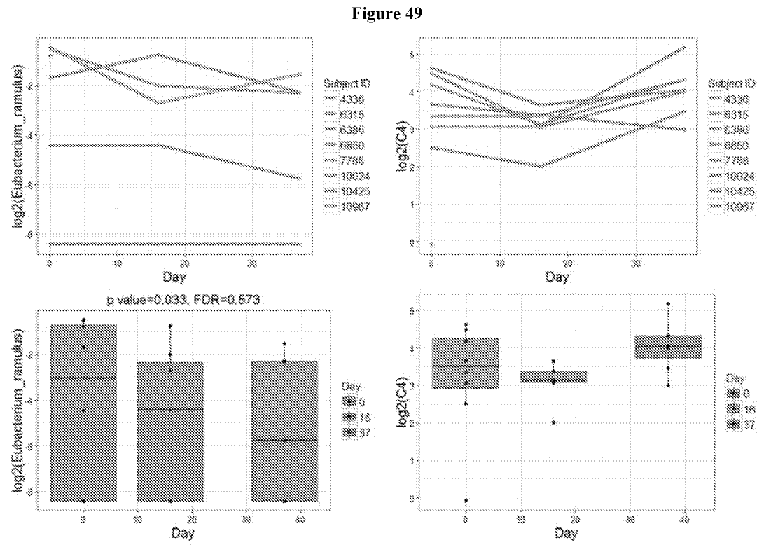

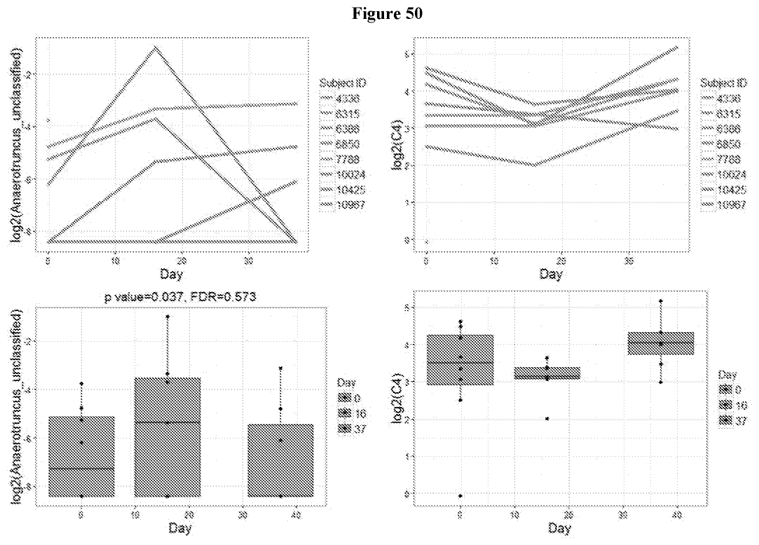

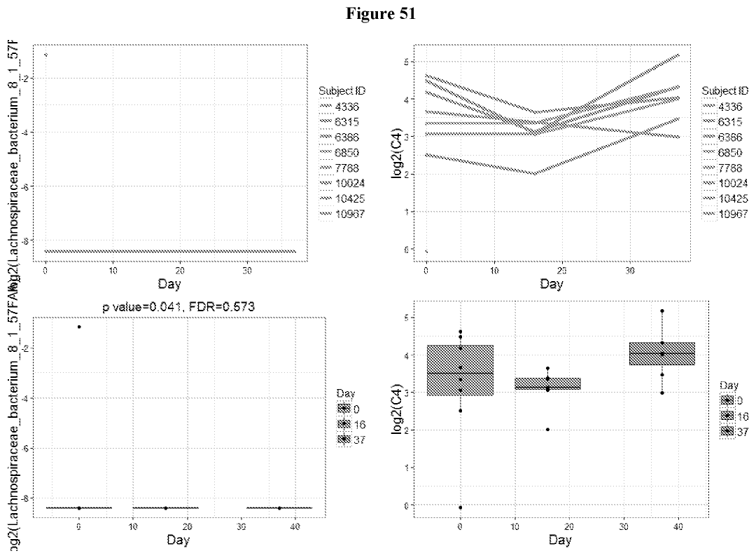

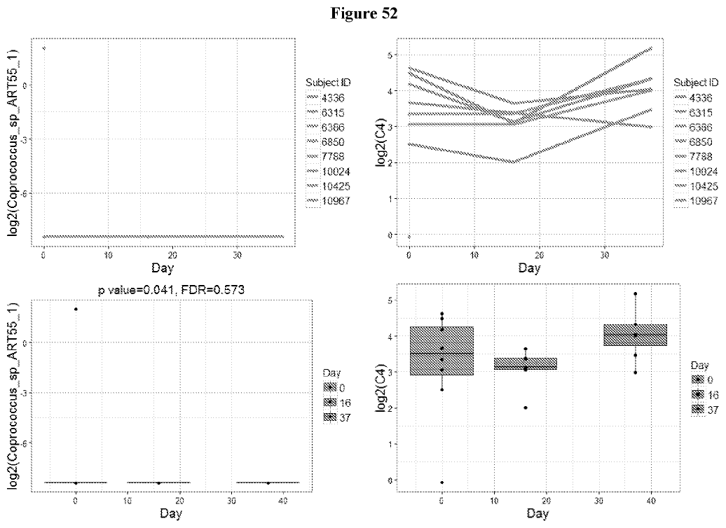

The time effect of OCA treatment on gut microbiome species was studied at three OCA dose group separately (as shown in Table 1). Analysis of the stool samples revealed the different abundance of various species (Friedman rank test, p<0.05) over time (Table 2). Table 2 shows that Lactobacillus casei paracasei and Streptococcus thermophilus, gram-positive organisms that are sensitive to growth inhibition by bile acids, were differentially abundant over time in all three OCA dosage groups (FIG. 1) and their numbers decreased upon discontinuation of OCA. Similar association was not observed with any of the gram-negative organisms. Additionally, FIG. 2-FIG. 5, FIG. 19-FIG. 25, and FIG. 45-FIG. 52 show a selected list of species based on the analysis of 5 mg OCA dose group, and FIG. 13-FIG. 15 and FIG. 26-FIG. 44 show a selected list of species based on the analysis of 10 mg OCA dose group.

TABLE 2

| | gram-positive (+) | | P value (FDR) | |
|---|---|---|---|---|
| species | gram-negative (−) | OCA 5 mg | OCA 10 mg | OCA 25 mg |
| Lactobacillus_casei_paracasei | + | 0.006(0.244) | 0.018(0.563) | 0.023(0.453) |
| Steptococcus_thermophilus | + | 0.004(0.244) | 0.002(0.185 | 0.016(0.453) |
| Lactococcus_lactis | + | 0.016(0.296) | 0.006(0.343) | 0.692(0.794) |
| Odoribacter_splanchnicus | − | 0.039(0.419) | 0.115(0.626) | 0.867(0.896) |
| Alistipes_shahii | − | 0.022(0.296) | 0.957(1) | 0.368(0.573) |
| Clostridium_symbiosum | | 0.016(0.296) | 0.066(0.626) | 0.273(0.573) |
| Ruminococcus_torques | | 0.005(0.244) | 0.276(0.646) | 0.867(0.896) |
| Ruminococcus_bromii | | 0.038(0.419) | 0.717(0.936) | 0.368(0.573) |
| Coprobacilllus_unclassified | | 0.009(0.276) | 0.282(0.646) | 0.273(0.573) |
| Escherichia_coli | − | 0.022(0.296) | 0.751(0.936) | 0.959(0.972) |
| Akkermansia_muciniphila | − | 0.022(0.296) | 0.761(0.936) | 0.135(0.48) |
| Bacteroides_ovatuds | − | 0.311(0.534) | 0.018(0.563) | 0.074(0.467) |
| Veillonella_unclassified | − | 0.109(0.534) | 0.042(0.626) | 0.076(0.467) |
| Bifidobacterium_bifidum | | 0.368(0.534) | 0.368(0.646) | 0.05(0.453) |
| Bacteroides_dorei | | 0.607(0.729) | 0.135(0.626) | 0.022(0.453) |
| Paraprevotella_clara | | 0.607(0.729) | 0.097(0.626) | 0.039(0.453) |
| Paraprevotella_unclassified | | 0.607(0.729) | 0.097(0.626) | 0.039(0.453) |
| Steptococcus_parasanguinis | | 0.148(0.534) | 0.331(0.646) | 0.04(0.453) |
| Eubacterium_hallii | | 0.074(0.534) | 0.819(0.952) | 0.011(0.453) |
| Eubacterium_rectale | | 0.568(0.729) | 0.317(0.646) | 0.042(0.453) |
| Anaerostipes_hadrus | | 0.282(0.534) | 0.066(0.626) | 0.04(0.453) |
| Coprococcus_catus | | 0.368(0.534) | 0.174(0.646) | 0.042(0.453) |
| Dorea_longicatena | | 0.607(0.729) | 0.311(0.646) | 0.03(0.453) |

### Example 3: Time Effect of OCA Treatment on Gene and Pathways

### Gene Differential Abundance Analysis

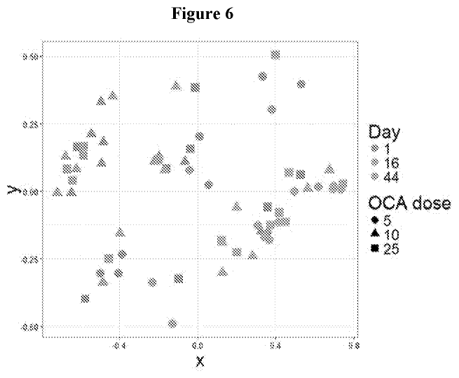

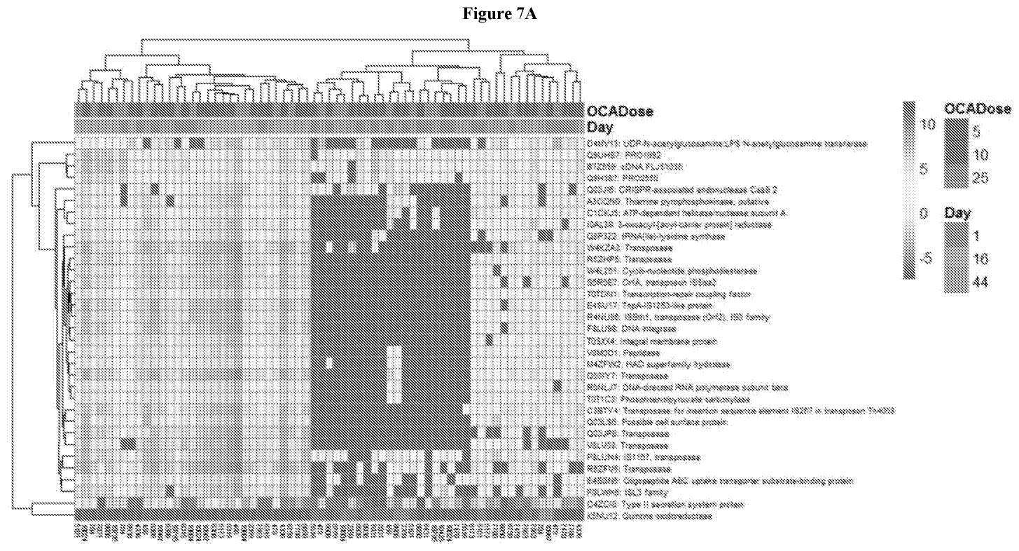

The time and dose effect of OCA treatment on gene abundance was studied using repeated measure ANOVA (see Example 1). No OCA dose effect or OCA dose×day interaction was observed on the dataset (repeated measure ANOVA, FDR<0.05). Therefore, 112 genes were identified differentially abundant over time (repeated measure ANOVA, FDR<0.05 for time effect). Table 3 lists the 33 most differentially abundant genes (repeated measure ANOVA, FDR<0.01 for time effect). The MDS plot (FIG. 6) and the heat map (FIG. 7A) of the 33 most differential abundance genes (repeated measure ANOVA, FDR<0.01 for time effect) show clear separation of samples among days. Gene representation showed a robust abundance of tranposases likely indicative of mobile DNA elements in the genomes of the same gram-positive bacterial taxa (e.g., Streptococcus) that increase in representation with the administration of OCA. Additionally, glycosyl transferase genes that may contribute to exopolysaccharide formation in lactic acid bacteria were significantly increased in the presence of OCA.

TABLE 3

| | FDR | | |
|---|---|---|---|
| Gene (Uniref50) | dose | day | dose:day |
| R5ZFV6: Transposase | 0.892 | 1.56e−04 | 0.892 |
| Q03JP8: Transposase | 0.597 | 2.61e−03 | 0.597 |
| C4ZCI8: Type II secretion system protein | 0.951 | 3.25e−03 | 0.951 |
| Q03IY7: Transposase | 0.840 | 3.25e−03 | 0.840 |
| SSR0E7: OrfA transposon ISSsa2 | 0.723 | 3.25e−03 | 0.723 |
| F8LUN4: IS1167, transposase | 0.840 | 4.46e−03 | 0.840 |
| B7Z559: cDNA FLI1035 | 0.901 | 4.61e−03 | 0.901 |
| Q03JI6: CRISPR-asscociated endonuclease | 0.942 | 4.61e−03 | 0.942 |
| Cas9 2 | | | |
| M4ZFW2: HAD superfamily hydrolase | 0.840 | 5.25e−03 | 0.840 |
| R5ZHP5: Transposase | 0.714 | 5.39e−03 | 0.714 |
| D4MV13: UDP-N-acetylglucosamine:LPS | 0.840 | 5.69e−03 | 0.840 |
| N-acetylglucoseamine transferase | | | |
| E45U17: Tnp-IS1253-like protein | 0.840 | 5.69e−03 | 0.840 |
| I0AL38: 3-oxoacyl-[acyl-carrier protein] | 0.840 | 5.69e−03 | 0.840 |
| reductase | | | |
| Q9H387: PRO2550 | 0.894 | 5.69e−03 | 0.864 |
| T0SXX4: integral membrane protein | 0.597 | 5.69e−03 | 0.597 |
| T0T1C3: Phosphoenolpyruvate carboxylase | 0.840 | 5.69e−03 | 0.840 |
| A3CQN0: Thiamine pyrophosphokinase, | 0.876 | 6.21e−03 | 0.876 |
| putative | | | |
| C1CKJS: ATP-dependent helicase/ | 0.840 | 6.21e−03 | 0.840 |
| nuclease subunit A | | | |
| R0NLJ7: DNA-directed RNA polymerase | 0.840 | 6.21e−03 | 0.840 |
| subunit | | | |
| R4NU86: ISSth1 transposase (Orf2), | 0.723 | 6.21e−03 | 0.723 |
| IS3 family | | | |
| T0TDN1: Transcription-repair coupling | 0.762 | 6.21e−03 | 0.762 |
| factor | | | |
| V8M0D1: Peptidase | 0.762 | 6.21e−03 | 0.762 |
| F8LU98: DNA integrase | 0.840 | 6.48e−03 | 0.840 |
| Q03LS6: Possble cell surface protein | 0.840 | 6.48e−03 | 0.840 |
| V8LV03: Tranposase | 0.657 | 6.48e−03 | 0.657 |
| X5NU12: Quinone oxidoreductase | 0.840 | 6.48e−03 | 0.840 |
| C38TY4: Transposase for insertion | 0.762 | 7.31e−03 | 0.762 |
| sequence element | | | |
| IS257 in transposon Tn4003 | | | |
| W4L251: Cyclo-nucleotide | 0.840 | 9.28e−03 | 0.840 |
| phosphodiesterase | | | |
| E4SSN6: Oligopeptide ABC uptake | 0.840 | 9.78e−03 | 0.840 |
| transporter substrate-binding protein | | | |
| F8LWK6: ISL3 family | 0.762 | 9.78e−03 | 0.762 |
| Q8P322: tRNA(IIe)-lysidine synthase | 0.840 | 9.78e−03 | 0.840 |
| Q91UH57: PRO1992 | 0.942 | 9.78e−03 | 0.942 |
| W4KZA3: Transposase | 0.840 | 9.78e−03 | 0.840 |

### MetaCyc Pathway Analysis

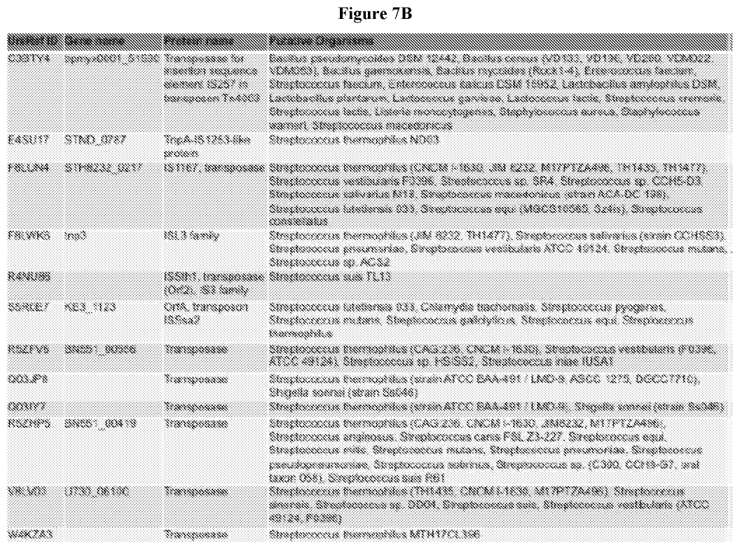

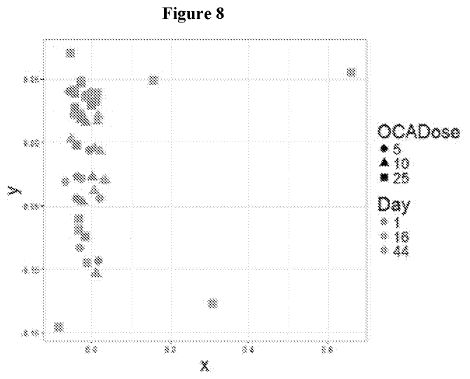

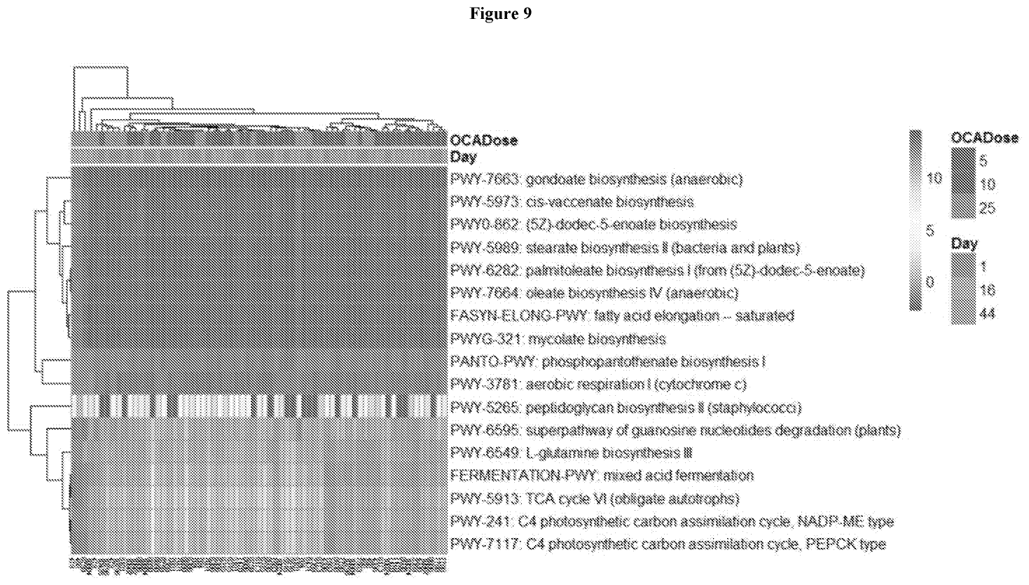

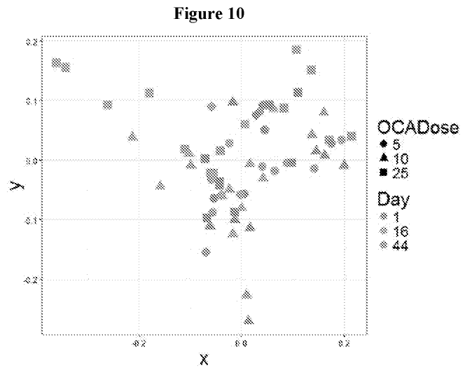

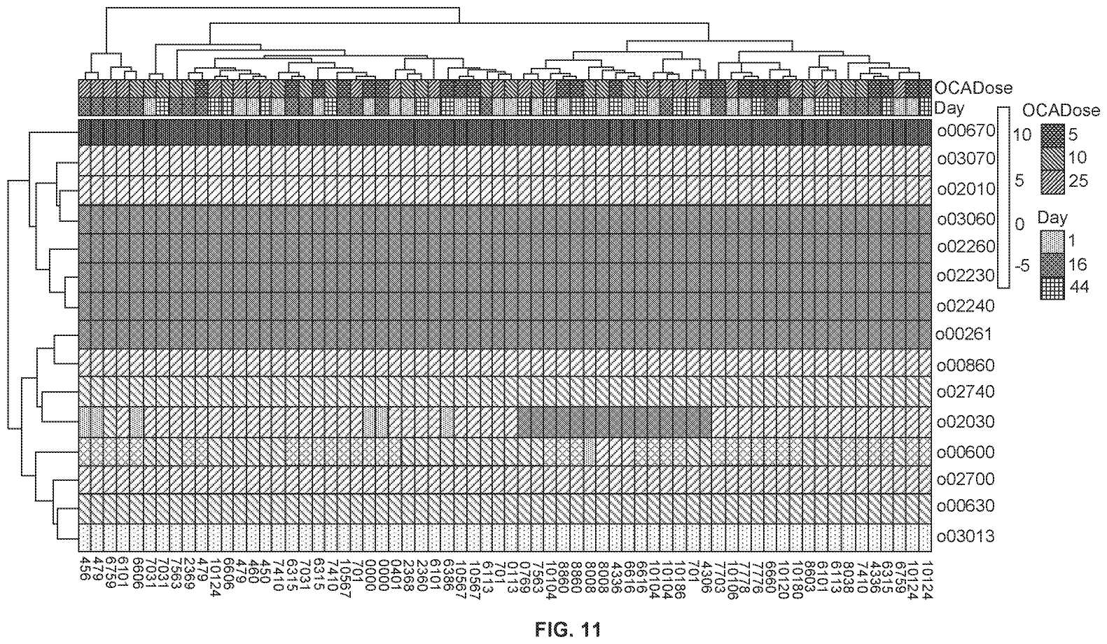

For each of the 515 MetaCyc pathways, the time and dose effect of OCA treatment was studied using repeated measure ANOVA (see Example 1). No OCA dose effect or OCA dose×day interaction was observed on the dataset (repeated measure ANOVA, FDR<0.05). 41 out of 515 MetaCyc pathways were identified differentially abundant over time (repeated measure ANOVA, FDR<0.05 for time effect). Table 4 shows the 17 most differentially abundant pathways (repeated measure ANOVA, FDR<0.01 for time effect). However, MDS plot (FIG. 8) and heat map (FIG. 9) of the 17 most differentially abundant MetaCyc pathways do not show clear separation of samples among days.

TABLE 4

| | | FDR | | |
|---|---|---|---|---|
| Pathway (MetaCyc) | * | dose | day | dose:day |
| PWY-3781: aerobic respiration I | ↓ | 0.468 | 7.03e−04 | 0.468 |
| (cytochrome c) | | | | |
| PWY-5989: stearate biosynthesis II | ↓ | 0.739 | 7.03e−04 | 0.739 |
| (bacteria and plants) | | | | |
| FASYN-ELONG-PWY: fatty acid | ↓ | 0.843 | 1.66e−03 | 0.843 |
| elongation - saturated | | | | |
| FERMENTATION-PWY: mixed | ↑ | 0.766 | 1.66e−03 | 0.766 |
| acid fermentation | | | | |
| PWY-241: C4 photosynthetic | ↑ | 0.818 | 1.66e−03 | 0.818 |
| carbon assimilation cycle, NADP-ME | | | | |
| type | | | | |
| PWV-5265: peptidoglycan biosysthesis | ↑ | 0.332 | 1.66e−03 | 0.332 |
| II (staphylocci) | | | | |
| PWY-5913: TCA cycle VI (obligate | ↑ | 0.766 | 1.66e−03 | 0.766 |
| autotrophs) | | | | |
| PWY-6282: palmitoleate biosynthesis I | ↓ | 0.879 | 1.66e−03 | 0.879 |
| (from (SZ)-dodec-5-enoate) | | | | |
| PWY-6595: superpathway of guanosine | ↑ | 0.468 | 1.66e−03 | 0.468 |
| nucleotides degradation (plants) | | | | |
| PWY-7117: C4 photosynthetic carbon | ↑ | 0.797 | 1.66e−03 | 0.797 |
| assimilation cycle, PEPCK type | | | | |
| PWY-7664:1 oleate biosynthesis IV | ↓ | 0.885 | 1.66e−03 | 0.885 |
| (anaerobic) | | | | |
| PWY0-862: (5Z)-dodec-5-enoate | ↓ | 0.958 | 1.66e−03 | 0.958 |
| biosynthesis | | | | |
| PWYG-321: mycolate biosynthesis | ↓ | 0.879 | 1.66e−03 | 0.879 |
| PANTO-PWY: phosphopantotnenate | ↓ | 0.365 | 2.10.e−03 | 0.365 |
| biosynthesis | | | | |
| PWY-5573: cis-vaccenate biosynthesis | ↓ | 0.912 | 2.45e−03 | 0.912 |
| PWY-6549: L-glutamine biosynthesis III | ↑ | 0.765 | 3.47e−03 | 0.765 |
| PWY-7663: gondoate biosynthesis | ↓ | 0.571 | 7.56e−03 | 0.571 |
| (anaerobic) | | | | |

*“↑” means the pathway is up-regulated on day 16
*“↓” means the pathway is down-regulated on day 16

### KEGG Pathway Analysis

For each of the 66 KEGG pathways, the time and dose effect of OCA treatment was studied (see Example 1). No OCA dose or dose×day interaction was observed (repeated measure ANOVA, FDR<0.05). 26 KEGG pathways were identified differentially abundant over time (repeated measure ANOVA, FDR<0.05 for time effect). Table 5 shows the 15 most differential abundant KEGG pathways (repeated measure ANOVA, FDR<0.01 for time effect).

TABLE 5

| Path ID | Name | * | dose | day | dose:day |
|---|---|---|---|---|---|
| ko00600 | Sphingolipid metabolism | — | 0.617 | 3.59e−05 | 0.617 |
| ko03060 | Protein export | ↑ | 0.617 | 7.64e−04 | 0.617 |
| ko02030 | Bacterial chemotaxis | ↓ | 0.893 | 1.70e−03 | 0.893 |
| ko00260 | Glycine, serine and threonine | ↑ | 0.617 | 3.53e−03 | 0.617 |
| | metabolism | | | | |
| ko03013 | RNA transport | ↓ | 05.87 | 3.53e−03 | 0.587 |
| ko00230 | Purine metabolism | ↑ | 0.683 | 7.70e−03 | 0.683 |
| ko00240 | Pyrimidine metabolism | ↑ | 0.683 | 7.89e−03 | 0.683 |
| ko00670 | One carbon pool by folate | ↑ | 0.832 | 7.89e−03 | 0.832 |
| ko03070 | Bacterial secretions system | ↑ | 0.617 | 8.46e−03 | 0.617 |
| ko00061 | Fatty acid biosynthesis | ↓ | 0.683 | 8.68e−03 | 0.683 |
| ko00630 | Glyoxylate and dicarboxylate | ↓ | 0.617 | 8.68e−03 | 0.617 |
| | metabolism | | | | |
| ko02010 | ABC transporters | ↑ | 0.650 | 8.68e−03 | 0.650 |
| ko00740 | Riboflavin metabolism | ↓ | 0.617 | 9.82e−03 | 0.617 |
| ko00780 | Biotin metabolism | ↓ | 0.617 | 9.82e−03 | 0.617 |
| ko00860 | Porphyrin and clorophyll | ↓ | 0.683 | 9.82e−03 | 0.683 |
| | metabolism | | | | |

*“↑” means the pathway is up-regulated on day 16
*“↓” means the pathway is down-regulated on day 16

### FGF19 Analysis

Association of FGF19 and Microbiome Species

The relationship of FGF19 level with the species change was studied using GEE model (see Example 1) on two OCA dose groups (5 mg and 10 mg) separately. Table 6 shows the species associated with FGF19 change (GEE, p<0.05) in each OCA dose group. However, no overlap was found between two dose groups on species level.

TABLE 6

| | P value (FDR) | |
|---|---|---|
| Species | OCA dose = 5 mg | OCA dose = 10 mg |
| Alistipes_shahii | 3.73e−11(4.51e−09) | 0.097(0.577) |
| Alistipes_indistinctus | 5.93e−07(3.59e−05) | 0.232(0.635) |
| Escherichia_coli | 3.64e−04(0.015) | 0.427(0.701) |
| Bacteroides_vulgartus | 6.37e−04(0.019) | 0.712(0.852) |
| Parabacteroides_merdae | 3.06e−03(0.066) | 0.247(0.635) |
| Alistipes_putredinis | 3.39e−03(0.066) | 0.395(0.682) |
| Flavonifractor_plautii | 3.85e−03(0.066) | 0.522(0.755) |
| Clostridum_asparagiforme | 5.70e−03(0.086) | 0.55(0.761) |
| Oscillibacter_sp_KLE_1745 | 6.71e−03(0.09) | 0.298(0.635) |
| Lachnospiraceae_ | 0.013(0.154) | 0.599(0.761) |
| bacterium_5_1_63FAA | | |
| Subdoligranulum_sp_4_3_ | 0.014(0.154) | 0.713(0.852) |
| 54A2FAA | | |
| Alistipes_sp_AP11 | 0.043(0.429) | 0.581(0.761) |
| Lactobacillus_casei_paracasei | 0.694(0.858) | 2.27e−08(2.79e−06) |
| Streptococcus_thermophilus | 0.39(0.674) | 5.11e−03(0.314) |
| Coprococcus_comes | 0.744(0.883) | 9.14e−03(0.316) |
| Clostridum_bartlettii | 0.306(0.655) | 0.01(0.316) |
| Oscillibacter_sp_KE_1728 | 0.786(0.897) | 0.017(0.482) |
| Clostridium_leptum | 0.517(0.727) | 0.029(0.52) |
| Ruminococcus_obeum | 0.893(0.948) | 0.03(0.52) |
| Oscillibacter_unclassified | 0.32(0.655) | 0.037(0.527) |
| Holdemania_filiformis | 0.155(0.622) | 0.039(0.527) |
| Bifidobacterium_longum | 0.467(0.727) | 0.045(0.527) |

Association of FGF19 and Genes

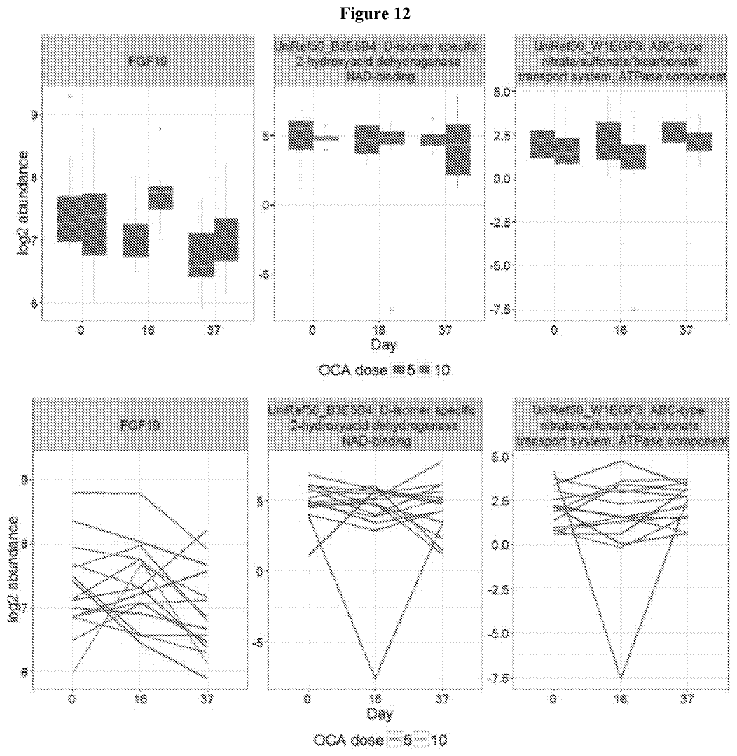

GEE model was applied to study the association of FGF19 and the most varying gene families. For each of the 2294865 most varying genes, association of FGF19 and gene abundance were analyzed in each OCA dose group separately (see Example 1). Table 7 shows the number of significant genes identified at different cut-off, and Table 8 shows the 37 genes significantly associated with FGF19 (GEE, p<0.01) in both OCA dose groups. FIG. 12 presents the association of FGF19 with the two genes (D-isomer specific 2-hydroxyacid dehydrogenase NAD-binding and ABC-type nitrate/sulfonate/bicarbonate transport system, ATPase component) over time at OCA dose of 5 mg or 10 mg.

TABLE 7

| OCA dose group | P < 0.05 | P < 0.01 | FDR < 0.05 | FDR < 0.01 |
|---|---|---|---|---|
| 5 mg | 4653 | 1753 | 505 | 274 |
| 10 mg | 4775 | 1824 | 427 | 202 |
| Overlap | 288 | 37 | 2 | 1 |

TABLE 8

| | P value(FDR) | |
|---|---|---|
| Gene (Uniref50) | OCA dose = 5 mg | OCA dose = 10 mg |
| W1EGF3: ABC-type | 3.52e−06(1.08e−03) | |
| nitrate/sulfonate/bicarbon- | | |
| ate transport system, | | |
| ATPase component | | |
| B3E5B4: D-isomer specific | 8.42e−05(0.014) | 6.95e−(0.015) |
| 2-hydroxyacid dehydroge- | | |
| nase NAD-binding | | |
| C1FV51: NLP/P60 family | 3.52e−04(0.038) | 4.20e−04(0.05) |
| protein | | |
| D6DM40: Predicted | 1.98e−04(0.026) | 5.25e−04(0.058) |
| membrane protein | | |
| C8WHT7: Peptide deformy- | 7.94e−04(0.068) | 5.36e−04(0.058) |
| lase | | |
| A9BHQ5: Indole-3-glycerol | 3.60e−09(5.13e−06) | 9.76e−04(0.086) |
| phosphate synthase | | |
| I2F921: Transketolase | 1.15e−03(0.086) | 1.44e−06(7.20e−04) |
| subunit A | | |
| S0EXL8: Nicotinate- | 1.19e−03(0.088) | 5.65e−04(0.06) |
| nucleotide pyrophosphory- | | |
| lase [carboxylating] | | |
| E3Z967: PTS system, | 2.15e−06(7.36e−04) | 1.09e−03(0.091) |
| Lactose/Cellobiose specific | | |
| IIB subunit subfamily | | |
| M4LFP2: Catabolite control | 1.85e−05(4.22e−03) | 1.35e−03(0.103) |
| protein A | | |
| F7KYC8: Putative anaerobic | 1.26e−04(0.018) | 1.62e−03(0.112) |
| sulfite reductase, subunit A | | |
| A2RCF8: Ribonuclease P | 1.43e−03(0.099) | 2.17e−03(0.131) |
| protein component | | |
| G9YR12: MATE efflux | 2.45e−03(0.133) | 2.17e−03(0.131) |
| family protein | | |
| T2TXF2: Lysozyme-like | 2.97e−03(0.146) | 7.05e−05(0.015) |
| family protein | | |
| RSBPF1: Ser/Thr phospha- | 1.49e−05(3.58e−03) | 2.65e−03(0.417) |
| tase family protein | | |
| J9C45: Membrane protein | 3.29e−03(0.152) | 1.07e−03(0.091) |
| F8F3Z5: Prevent-host-death | 2.38e−04(0.029) | 3.17e−03(0.159) |
| protein | | |
| E1XGE4: ABC transporter | 3.98e−03(0.168) | 3.01e−03(0.156) |
| B8FWA1: Amino acid- | 2.12e−04(0.027) | 3.64e−03(0.169) |
| binding ACT domain protein | | |
| R6VDI3: Anti-sigma factor | 4.12e−03(0.17) | 3.09e−03(0.158) |
| F2NAP1: Transcription | 4.26e−03(0.174) | 4.07e−05(0.01) |
| elongation factor GreA | | |
| W25UH9: Addiction module | 5.16e−03(0.195) | 3.62e−03(0.169) |
| toxin, RelE/StbE family | | |
| S6C145: Cytoplasmic | 2.25e−04(0.028) | 5.23e−03(0.201) |
| chaperone | | |
| G9YUN4: Sigma-70 region 2 | 2.28e−04(0.028) | 5.31e−03(0.202) |
| F9Z5H0: RNA polymerase, | 8.45e−05(0.014) | 5.81e−03(0.212) |
| sigma-24 subunit, EACF | | |
| subfamily | | |
| D6ZJ98: GtrA-like protein | 5.82e−03(0.212) | 1.61e−04(0.026) |
| D3AQF9: Cna protein B-type | 5.94e−03(0.214) | 3.07e−03(0.157) |
| domain protein (Fragment) | | |
| W1XYY2: Integrase, catalytic | 1.48e−09(2.61e−06) | 6.18e−03(0.219) |
| region (Fragment) | | |
| D4JEC7: ATPase components | 6.40e−03(0.223) | 5.61e−04(0.06) |
| of various ABC-type transport | | |
| systems, contai duplicated | | |
| ATPase | | |
| C0EAX2: Abi-like protein | 6.90e−03(0.232) | 5.43e−03(0.205) |
| C7N8C1: ATP synthase | 6.96e−03(0.233) | 6.72e−03(0.229) |
| subunit a | | |
| L0FUP0: HIT family hydro- | 6.97e−03(0.233) | 1.50e−03(0.15) |
| lase, diadenosine tetraphos- | | |
| phate hydrolase | | |
| T0TAPS: Biotin-requiring | 7.54e−03(0.248) | 2.75e−03(0.15) |
| enzyme | | |
| XoVZC2: Marin sediment | 7.81e−03(0.252) | 1.12e−03(0.092) |
| metagenome DNA, contig: | | |
| S01H1_521116 (Fragment) | | |
| D4JVW2: Predicted Zn | 1.96e−03(0.117) | 8.81e−03(0.262) |
| peptidase | | |
| C8WG72: Hydro-lyase | 8.41e−03(0.263) | 1.55e−03(0.11) |
| Fe—S type, tartrate/ | | |
| fumarate subfamily, alpha | | |
| subunit | | |
| X1VFA2: Marine sediment | 3.11e−03(0.149) | 9.44e−03(0.272) |
| metagenome DNA, contig: | | |
| S12H4_S11451 (Fragment) | | |

### Association of FGF19 and MetaCyc Pathways

For each of the 515 MetaCyc pathways, GEE model was applied to study the association of FGF9 and MetaCyc pathway abundance (see Example 1). Table 9 shows the number of significant MetaCyc pathways identified at different cut-off, and Table 10 shows the MetaCyc pathways significantly associated with FGF19 in both OCA dose groups.

TABLE 9

| OCA dose group | P < 0.05 | P < 0.01 | FDR < 0.05 | FDR < 0.01 |
|---|---|---|---|---|
| 5 mg | 56 | 27 | 14 | 8 |
| 10 mg | 67 | 32 | 23 | 7 |
| Overlap | 4 | 0 | 0 | 0 |

TABLE 10

| | P value(FDR) | |
|---|---|---|
| | OCA dose = | OCA dose = |
| MetaCyc Pathway | 5 mg | 10 mg |
| POLYISOPRENSYN-PWY: | 0.044(0.409) | 0.046(0.354) |
| polyisoprenoid biosynthesis (E. coli) | | |
| PWY-6270: isoprene biosynthesis I | 0.043(0.409) | 5.28e−04(0.019) |
| PWY-7209: superpathway of pyrimidine | 0.032(0.328) | 1.03e−03(0.025) |
| ribonucleosides degradation | | |
| PWY66-422: D-galactose degradation V | 0.022(0.284) | 0.022(0.234) |
| (Leloir pathway) | | |

### C4 Analysis

Association of C4 and Microbiome Species

The association of C4 and microbiome species was analyzed on two OCA dose group separately. In 5 mg OCA group, 8 species was identified significantly (p<0.05) associated with C4 change over time (Table 11). In 10 mg OCA group, 17 species was identified significantly (p<0.05) associated with C4 change over time (Table 12). FIG. 13-FIG. 15 and FIG. 31-FIG. 44 show a selected list of species significantly associated with C4 in 10 mg OCA dose group. In addition, FIG. 45-FIG. 52 show a selected list of species significantly associated with C4 in 5 mg OCA dose group. Statistically-significant reciprocal association was observed between C4 levels and abundances of tow gram-positive small intestinal taxa, S. thermophilus and L. casei-paracasei supporting the hypothesis that suppression of endogenous production by OCA favors expansion of bile acid sensitive small intestinal gram-positive bacterial taxa.

TABLE 11

| species | P value | FDR |
|---|---|---|
| Bacteroides_uniformis | 1.61e−13 | 1.95e−11 |
| Escherichia_coli | 8.56e−03 | 5.18e−01 |
| Streptococcus_parasanguinis | 2.82e−02 | 5.73e−01 |
| Ruminococcus_gnavus | 3.21e−02 | 5.73e−01 |
| Eubacterium_ramulus | 3.29e−02 | 5.73e−01 |
| Anaerotruncus_unclassified | 3.70e−02 | 5.73e−01 |
| Lachnospiraceae_bacterium_8_1_57FAA | 4.10e−02 | 5.73e−01 |
| Coprococcus_sp_ART55_1 | 4.10e−02 | 5.73e−01 |

TABLE 12

| species | P value | FDR |
|---|---|---|
| Streptococcus_thermophilus | 8.36e−08 | 1.03e−05 |
| Lachnosoiraceae_bacterium_5_1_63FAA | 1.85e−07 | 1.14e−05 |
| Lactobacillus_casei_paracasei | 5.94e−04 | 2.44e−02 |
| Bifidobacterium_breve | 2.26e−03 | 6.96e−02 |
| Alistipes_putredinis | 4.54e−03 | 1.12e−01 |
| Lactococus_lactis | 1.34e−02 | 2.37e−01 |
| Streptococcus_salivarius | 1.49e−02 | 2.37e−01 |
| Subdoligranlum_unclassified | 1.74e−02 | 2.37e−01 |
| Lachnospiraceae_bacterium_3_1_57FAA_CT1 | 1.83e−02 | 2.37e−01 |
| Dorae_longicatena | 1.93e−02 | 2.37e−01 |
| Bacteroidales_bacterium_ph8 | 2.59e−02 | 2.86e−01 |
| Bifidobacterium_longum | 2.79e−02 | 2.86e−01 |
| Bacteroides_plebeius | 3.40e−02 | 3.22e−01 |
| Ruminococcus_obeum | 4.37e−02 | 3.42e−01 |
| Paraprevoteia_clara | 4.38e−02 | 3.42e−01 |
| Clostridium_spiroforme | 4.64e−02 | 3.42e−01 |
| Paraprevotella_unclassified | 4.84e−02 | 3.42e−01 |

### OCA Dose Effect

OCA Dose Effect on FGF19

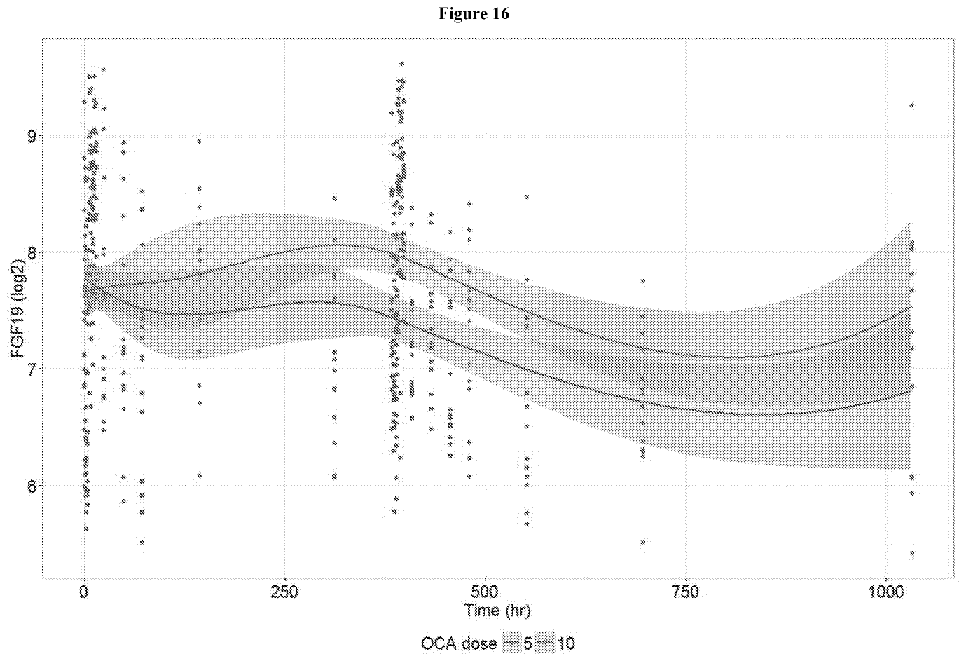

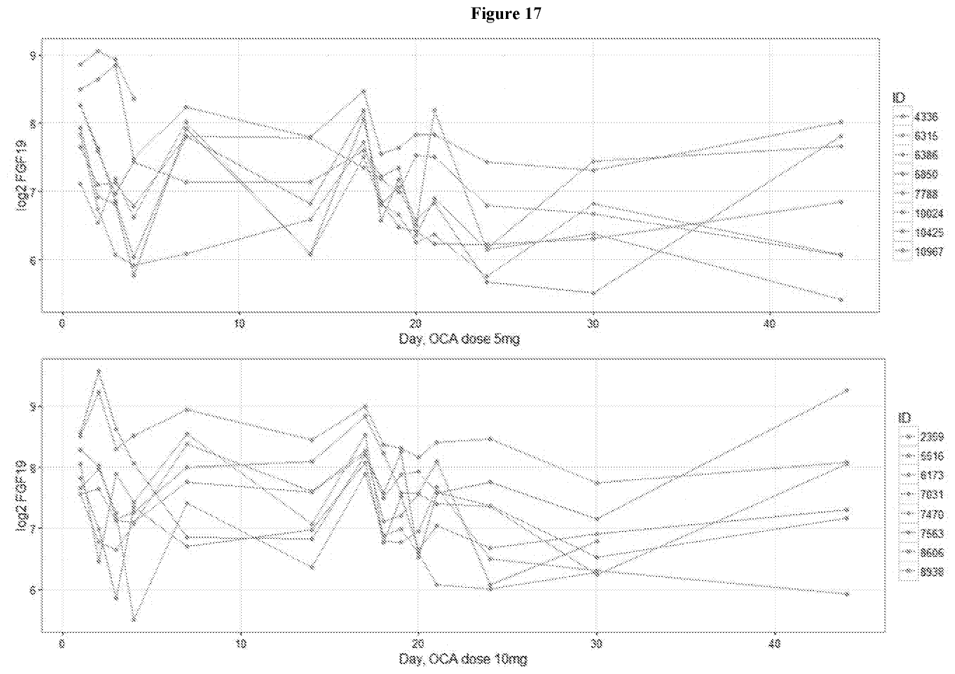

The dose and time (by hour) effect of OCA treatment on FGF19 level was analyzed using GEE model (see Example 1). The p value of time effect is 4.867×10−8, and the p value of time: OCA dose is 3.142×10−3. See also FIGS. 16 and 17.

OCA Dose Effect on Gut Microbiome Species

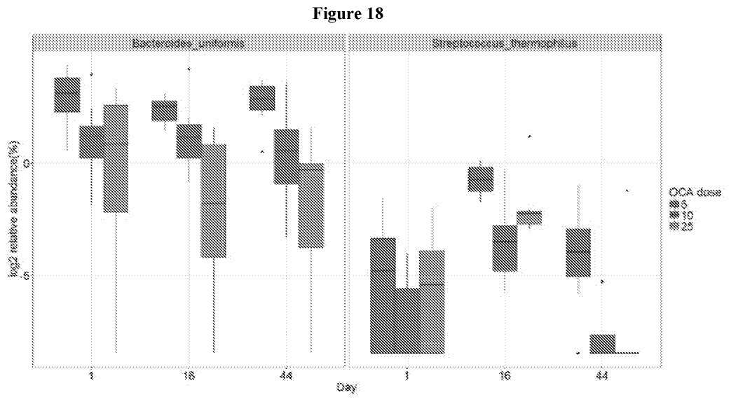

The OCA dose effect was studied in each time point separately. Kruskal-Wallis test was used to check the OCA dose effect on microbiome species abundance. Bacteroides uniformis and Streptococcus thermophilus were significantly different among dose groups (Kruskal-Wallis test, p<0.05) at both day 16 and day 37 (Table 13 and FIG. 18).

TABLE 13

| | P value(FDR) | | |
|---|---|---|---|
| species | day1 | day16 | day37 |
| Bacteroides_ovatus | 0.026(0.738) | 0.023(0.488) | 0.111(0.703) |
| Eubacterium_ventriosum | 0.007(0.477) | 0.051(0.54) | 0.042(0.703) |
| Bacteroides_uniformis | 0.063(0.738) | 0.007(0.223) | 0.006(0.703) |
| Streptococcus_thermophilus | 0.29(0.785) | 0.006(0.223) | 0.015(0.703) |
| Clostridium_asparagiforme | 0.035(0.738) | 0.118(0.54) | 0.303(0.837) |
| Clostridium_leptum | 0.031(0.738) | 0.089(0.54) | 0.948(0.991) |
| Clostridium_symbiosum | 0.038(0.738) | 0.293(0.747) | 0.677(0.896) |
| Anaerotruncus_colihominis | 0.007(0.477) | 0.071(0.54) | 0.578(0.883) |
| Bacteroides_thetaiotaomicron | 0.099(0.738) | 0.03(0.488) | 0.186(0.837) |
| Eubacterium_siraeum | 0.527(0.816) | 0.031(0.488) | 0.116(0.703) |
| Ruminococcus_torques | 0.064(0.738) | 0.006(0.223) | 0.078(0.703) |
| Oscillibacter_unclassified | 0.193(0.785) | 0.018(0.454) | 0.097(0.703) |
| Coprobacillus_unclassified | 0.399(0.799) | 0.004(0.223) | 0.421(0.837) |
| Parabacteroides_johnsonii | 0.127(0.761) | 0.121(0.54) | 0.029(0.703) |
| Roseburia_inulinivorans | 0.059(0.738) | 0.623(0.906) | 0.046(0.703) |
| Eubacterium_biforme | 0.092(0.738) | 0.08(0.54) | 0.039(0.703) |

Enzymes Associated with FGF19

Table 14 shows the genes associated with FGF19 that can be mapped to EC number.

TABLE 14

| | | P value (FDR) | |
|---|---|---|---|
| Gene(Uniref50 ID) | | OCA dose = 5 mg | OCA dose = 10 mg |
| B3E5B4: D-isomer specific | EC:1.1.1.29 | 8.42e−05(0.014) | 6.95e−05(0.015) |
| 2-hydroxyacid dehydrogenase | | | |
| NAD-binding | | | |
| C8WHT7: Peptide deformylase | EC:3.5.1.88 | 7.94e−04(0.068) | 5.36e−04(0.058) |
| A9BHQ5: Indole-3-glycerol | EC:4.1.1.48 | 3.60e−09(5.13e−06) | 9.76e−04(0.086) |
| phosphate synthase | | | |
| S0EXL8: Nicotinate-nucleotide | EC:2.4.2.19 | 1.19e−03(0.088) | 5.65e−04(0.06) |
| pyrophosphorylase [carboxylating] | | | |
| A2RCF8: Ribonuclease P protein | EC:3.1.26.5 | 1.43e−03(0.099) | 2.17e−03(0.131) |
| component | | | |
| C8WG72: Hydro-lyase, Fe-S type, | EC:4.2.1.2 | 8.41e−03(0.263) | 1.55e−03(0.11) |
| tartrate/fumarate subfamily, alpha | | | |
| subunit | | | |

### Example 4: Clinical Study

An open label, randomized, single dose and multiple dose trial to assess the pharmacokinetics of obeticholic acid (OCA) in 24 healthy male or female subjects aged 18 to 55 years receiving 5, 10 or 25 mg OCA was conducted. Stool specimens for microbiota genome testing were collected by subjects up to 2 days before Day 0 (T0), Day 13 or 14 (T1), and Day 37 (T2). The specimens were subject to statistical analysis to assess the following:
bacterial taxa and genes or pathways that change their abundances over time after OCA treatment
bacterial taxa or genes/functional groups that are associated with the FGF19 level
effects of different OCA dose levels on FGF19 level and microbiome compositions
change of fungal abundances and their link with FGF19 level after OCA treatment
enzyme commission numbers (EC) that show different abundances after OCA treatment and the ECs that are associated with FGF19 level

Shotgun metagenomic data were obtained from 24 subjects at the baseline T0, and from 22 subjects at the two following up data points (T1 and T2). Measurements of C4, FGF19 and other bile acid at three time points were also collected for statistical analyses at various taxonomic and functional levels.

The main analysis tool to quantify the composition of microbial communities is MetaPhlAn (Segata et al., Nat. Methods 9, 811 (2012)), which provides relative abundance estimates of the bacteria at different taxonomic levels. The overall change of microbiome compositions after OCA treatment using a distance-based PERMANOV A with three time points and OCA dose level as factors was examined, where weighted Jaccard distances were calculated for all pairs of samples and used as responses. An MDS plot was used for exploring any clusters in the data. Permutations was used to assess the statistical significance of change of microbiome compositions over time after OCA treatment. The same PERMANOVA framework was applied to test association between gut microbiome composition and FGF19 level using data from all data points, where FGF19 level was used as a continuous covariate and individual subjects were used as a strata variable in order to account for repeated measures.

The bacterial taxa that change their abundances over time after OCA treatment at various taxonomic levels, including species, genus and phylum levels were identified. A newly developed rank-based statistical tests to identify these taxa was applied. Compared to the standard paired rank sum test, the new test can effectively handle clumps of zeros that are often observed in taxonomic compositional data. The false discovery rate (FDR) controlling procedure of Benjamini-Hochberg was used to account for multiple comparisons. In addition, Friedman's rank-based repeated measurement ANOVA was implemented to examine whether there were bacterial taxa responding to OCA differently for different dose levels, with time points and OCA dose level as factors and bacterial taxon as a response variable. The generalized estimating equation (GEE) methods was applied to identify the bacterial taxa that were associated with the FGF19 level using data from all time points, where FGF19 levels over time were treated as outcomes, and the abundances of a given taxon over times were treated as time-dependent predictors. GEE was used to account for the dependency the data measured over different time points. Similar analysis was performed for changes of FGF19 level and changes of taxa abundances.

The HUMAnN package (Abubucker et al., PLoS Comput. Biol. 8, e1002358 (2012)) was used to determining the presence/absence and abundance of microbial pathways and the abundance of each orthologous gene family in a community from metagenomic data. Similar analyses were conducted to assess the taxonomic abundances in order to identify the microbial genes and metabolic pathways that changed their abundances over time using modified rank-based tests after OCA treatment. Using GEE, pathways and genes that were associated with the FGF19 level were identified, where FGF19 levels over time were treated as outcomes, and the abundances of a given gene or pathway over times were treated as time-dependent predictors. Benjamini and Horchberg was used to adjust for multiple comparisons.

Due to the importance of the secondary bile acid metabolism in OCA biology, the enzymes coded for the microbial genomes were studied. Blast with the metagenomic sequencing reads to KEGG enzyme commission numbers (ECs) was conducted. EC numbers do not specify enzymes, but enzyme-catalyzed reactions. If different enzymes (for instance from different organisms) catalyze the same reaction, then they receive the same EC number. The ECs that showed different abundances after OCA treatment using the modified Kruskal-Wallis rank test were identified to account for clumps of zeros often observed in such abundance data. GEE was used to identify the ECs that were associated with FGF19 level, where FGF19 levels over time were treated as outcomes, and the abundances of an EC over times were treated as time-dependent predictors. Benjamini and Hochberg was used for control for multiple comparisons.

Association between taxa abundances and EC numbers was explored to identify the bacterial taxa that were associated with certain enzyme reactions. Rank-based correlation analysis and heat map were applied to identify such associations.

Beside bacteria, the metagenomic data also provided a unique data source to study fungi and other microbes. The fungi abundances were assessed, and associated with OCA treatment and FGF19 level using similar analysis as the bacterial taxa outlined before.

The machine learning method Random Forests (Machine Learning 45, 5, (2001)) was applied to build a predictive model for FGF19 level after OCA treatment using taxa abundance, functional and pathway information and EC numbers measured at TO. Out-of-bag samples will be used to assess the prediction performance and the most important predictors will be identified. Alternatively, Random Forests was used to predict the change of FGF19 level at T1 or T2 from T0 based on changes of abundances of taxa and pathways.

Analysis of the shotgun metagenomic dataset evaluating the effect of OCA showed a consistent increase of low abundance gram positive organisms associated with the use of OCA (day 16) that decreased to baseline after OCA was discontinued (day 37). This pattern was inversely correlated to levels of plasma C4, a bile acid precursor. For example, two Gram-positive small intestinal taxa, Streptococcus thermophilus and Lactobacillus casei-paracasei, displayed statistically-significant associations after a correction for multiple comparisons with FDRs of 1.03e-5 and 2.44e-02, respectively. By contrast, no such association was observed with any of the Gram-negative organisms. Gene representation of mobile DNA elements (i.e., transposases) that is increased in representation with OCA treatment, was found in the genomes of various Streptococci spp. The lack of expansion of Gram-negative taxa with OCA treatment is consistent with this notion since most exhibit bile acid tolerance. The characterization of the bile acid sensitive Gram-positive organisms as constituents of the normal small intestinal microbiota is consistent with their relatively low abundance based on shotgun metagenomic reads as well as a number of species, including Streptococcus thermophilus and Lactobacillus casei-paracasei, being used in food manufacturing and also as commercially-available probiotics.

### Example 5: Effects of OCA on the Growth of Bacterial Strains Found in the Human Small Intestine

The toxicity of several bile acids to various gut microbiome species was examined. Representative species were exposed to different bile acids at a range of concentrations under either aerobic or anaerobic condition, and the growth of the species at these concentrations was measured by determining the optical density of the culture.

Lactobacillus casei CP was purchased from Custom Probiotics Inc. (Glendale, Calif.) as L. casei Custom Probiotic Powder (strain confirmed by Sanger sequencing of the 16S gene). Pediococcus pentosaceus KE-99 was purchased from Probiohealth (Beverly Hills, Calif.) as KE-99 LACTO Tablet (strain confirmed by Sanger sequencing of the 16S gene). L. casei 393, Streptoccocus thermophilus LMD-9, Akkermansia muciniphila Muc, and Veillonella parvula Te3 were purchased from the American Type Culture Collection (ATCC, Manassas, Va.). Lactococcus lactis NZ9000 was purchased from BOCA Scientific (Boca Raton, Fla.). Escherichia coli Nissle was obtained from Dr. Mark Goulian (University of Pennsylvania, Philadelphia, Pa.).

L. casei, P. pentasaceus, and L. lactis were grown in de Man, Rogosa, and Sharpe (MRS) medium (Anaerobe Systems, Morgan Hill, Calif.); E. coli was grown in lysogeny broth (LB) medium (Fisher Scientific, USA); S. thermophilus and A. muciniphila were grown in brain heart infusion (BHI) medium (Fisher Scientific, USA and Anaerobe Systems, Morgan Hill, Calif.), and; V. parvula was grown in reinforced clostridial medium (Fisher Scientific, USA). Aerobic cultures were incubated at 37° C.; anaerobic cultures were incubated at 37° C. in an anaerobic glove box (Coy Laboratories, Grass Lake, Mich.).

Glycochenodeoxycholic, glycocholic, and taurocholic acids were purchased from Sigma Aldrich (St. Louis, Mo.). Obeticholic acid was provided by Intercept Pharmaceuticals, Inc. (New York, N.Y.).

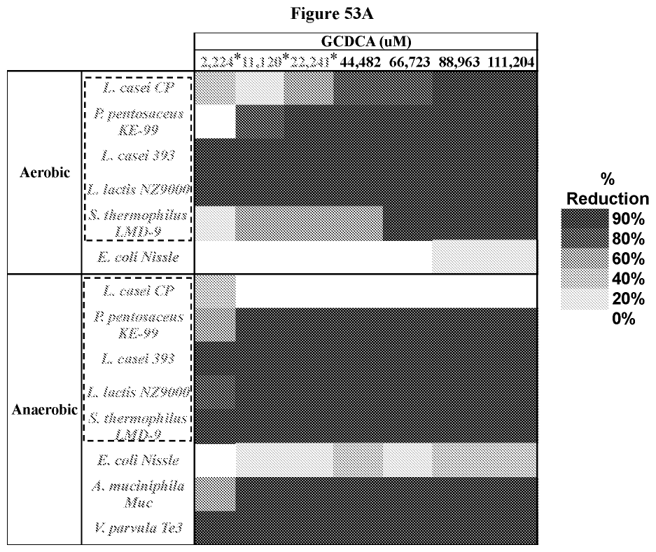

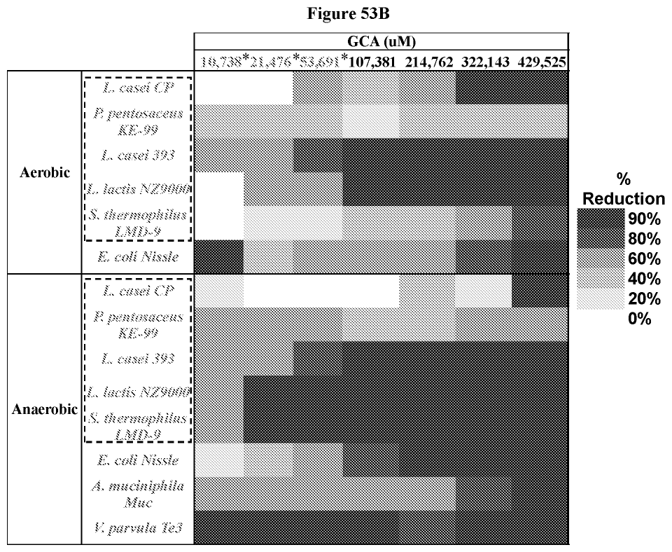

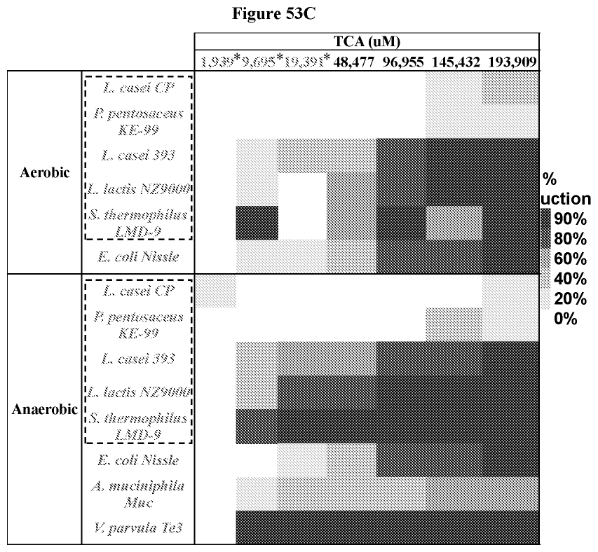

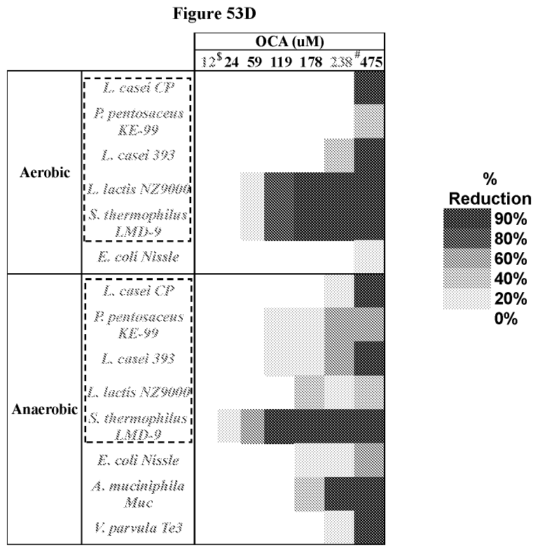

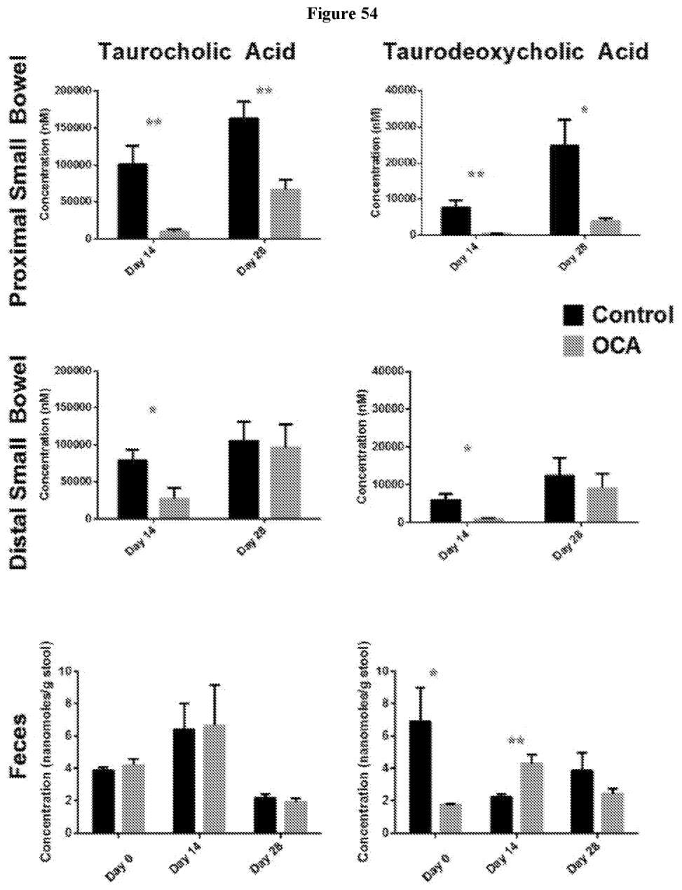

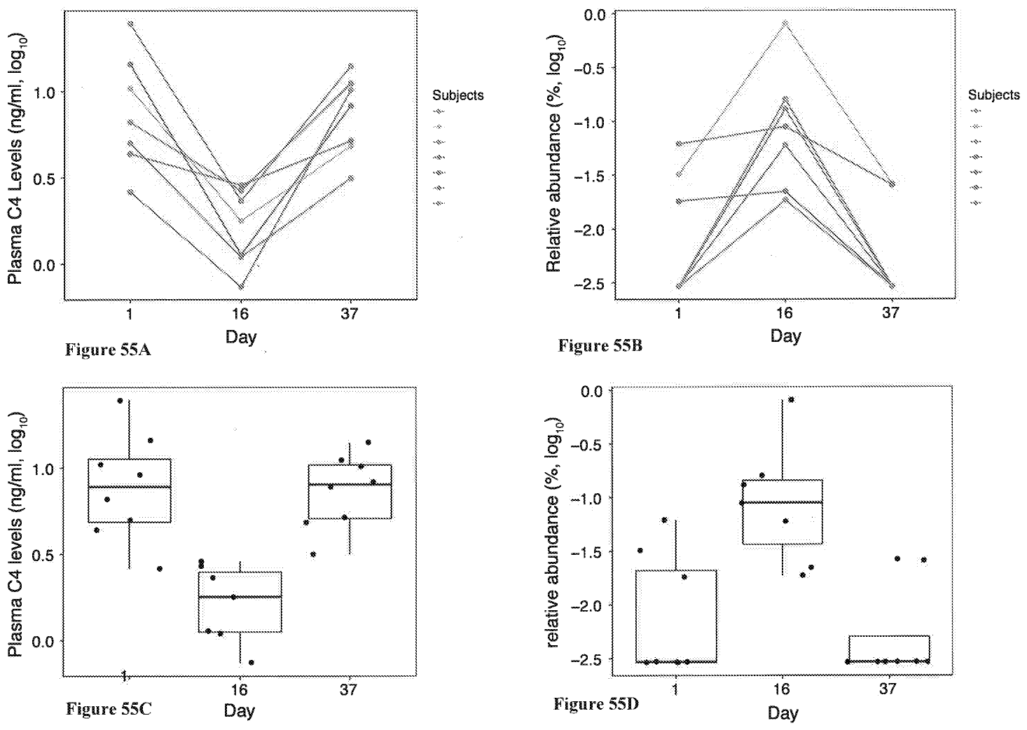

Inhibition of bacterial growth by bile acids was determined by the microbroth dilution method. Plates were prepared with 100 uL of medium containing the appropriate concentrations of bile acid. Wells were inoculated with 1 uL of an overnight culture, covered, and incubated overnight (3 days for A. muciniphila and V. parvula, which are slow growing organisms). Growth was measured via optical density at 630 nm, and measurements were zeroed against wells containing the appropriate bile acid level with no bacteria. All tests were performed in triplicate. Data is expressed as the percent (%) reduction in growth, which was calculated against controls (i.e., no bile acids). The responses of representative species to different concentrations of bile acids are illustrated by the heat maps shown in FIGS. 53A-53D.

As shown in FIGS. 53A-53C, differential growth inhibitory effects of conjugated (GCDCA and GCA) and unconjugated (TCA) bile acids on various strains of gram positivie bacteria were observed, indicating strain-specific effects. For example, the L. casei strain is relatively resistant to growth inhibition to all three endogenous bile acids, whereas the L. casei 393 strain is more sensitive. In general, the growth inhibitory effects of endogenous bile acids are more pronounced under anaerobic conditions relative to aerobic conditions. Since the redox potential of the small intestinal environment may be quite variable throughout its length, the effect of oxygen on the effects of bile acids on the growth bacteria needs to be considered. Moreover, the gram negative facultative anaerobe, E. coli, is generally more resistant to growth inhibition by endogenous bile acids under both aerobic and anaerobic conditions. These effects occurred at physiologically-relevant levels of endogenous biles in the human small intestine. Although high concentrations of OCA also has a growth inhibitory effect on several strains, no effect on any strain tested, either aerobically or anaerobically, at the calculated physiologic levels that would be encountered in the human small intestine (approximately 12 micromolar, FIG. 53D).

### Example 6: Effects of OCA on Bile Acid Levels Throughout the Length of the Intestinal Tract

The effect of OCA on the concentration of the primary and secondary taurine conjugated bile acids, taurocholic and taurodeoxycholic acids respectively, throughout the murine small intestine and in the feces is shown in FIG. 54. After 2 weeks of treatment with OCA (day 14), reduction of the levels of both bile acid was observed in both the proximal and distal small intestine, but not in the colon (feces). Two weeks after the cessation of OCA treatment (day 28), continued reduction of the levels of both bile acids was observed only in the proximal small intestine. These results indicate the regional inhibitory effects of OCA treatment on endogenous bile acid delivery into the small intestine. The specificity of these effects, limited to the small intestine but durable even after 2 weeks of OCA cessation, particularly in the proximal small intestine where the levels of these bile acids are the greatest, is consistent with the taxanomic effects of OCA on the gut microbiome, as a result of bile acid suppression specifically in the small intestine.

### Example 7: Increase in Specific Gram-Positive Facultative Anaerobic Bacterial Taxa Due to Suppression of Small Intestinal Bile Acid Levels by OCA Synthesis, Quantified by the Reduction in Plasma Levels of C4

Treatment with OCA Inhibits Synthesis of Endogenous Bile Acids and Increases the Relative Abundance of Several Low Level Gram-Positive Bacterial Taxa Detectable in Human Feces.

Activation of FXR by OCA and its subsequent effects on the small intestinal microbiota via bile acid-dependent mechanisms has revealed a range of novel opportunities, not only to improve precision medicine regarding the administration of small molecule agonists, but also to develop more reliable biomarkers and utilize currently available and future probiotics targeting the small intestine for the prevention and/or treatment of a variety of diseases.

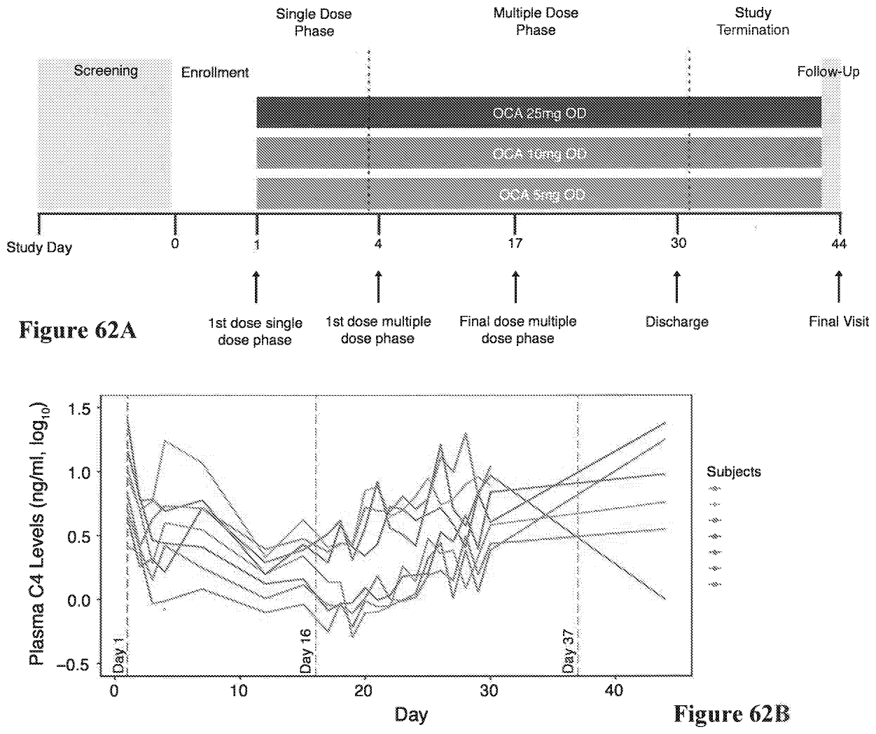

Twenty-four healthy subjects were randomly assigned to one of three dose groups (5 mg, 10 mg, or 25 mg OCA per day), with each dose group comprising eight subjects (four women and four men). A single oral OCA dose of 5 mg, 10 mg, or 25 mg tablets, depending on the treatment assignment, was administered on Day 1. In the multiple-dose phase, a single oral OCA dose of 5 mg, 10 mg, or 25 mg tablets, in accordance with the assigned treatment, was administered orally once daily for 14 days from Days 4-17. The patients remained at the study site until Day 30, and were followed up until the final visit on Day 44 (FIG. 62A). Fecal specimens were collected at baseline (Day 0), at the end of the multiple-dose phase (Day 17), and at the end of the study (Day 37). Pharmacokinetic blood samples were assessed pre-dose and on Days 1-3, 17, 35, 37, 39, and 44. Serial quantification of plasma C4, an intermediate in the synthesis of bile acids from cholesterol (Galman, et. al., Journal of Lipid Research 44, 859-866 (2003)), showed a time-dependent reduction (repeated measure ANOVA, p=4.77×10−5) in response to OCA treatment (FIG. 62B). Therefore, plasma C4 levels are a reliable dynamic indicator of the host response to FXR activation by OCA, that leads to the suppression of endogenous bile acid synthesis (Hirschfield et al., Gastroenterology 148, 751-761 e758 (2015); Mudaliar et al., Gastroenterology 145, 574-582 e571 (2013)). Features of the gut microbiome were matched to the variations in plasma levels of C4, that were maximally suppressed on Day 16 of the study relative to Day 0 (pre-OCA dose) and returned to normal by Day 37 (maximal time off OCA) (FIGS. 55A and 55C). 15 identified bacterial species showed a time-dependent correlation with C4 levels (generalized estimation equation (GEE), p<0.05; Table 15); five of these species remained statistically significant after correction for multiple comparisons (FDR<0.05). The time-dependent effect of OCA on Streptococcus thermophilus, the species showing the greatest increase, was striking, as this is a relatively low abundance taxon in the fecal microbiota, which, in most subjects, was undetectable without OCA treatment (FIGS. 55B and 55D). With two exceptions, Gram-positive bacterial genera showed increased abundances following OCA treatment. The abundances of all Gram-negative bacteria decreased as shown in Table 15.

Table 15: Correlation of bacterial species with alterations in plasma C4 change over time. 15 Species significantly (GEE, P value<0.05) correlated with plasma C4 levels were identified from subjects treated with 10 mg of OCA.

TABLE 15

| Phylum | Species | P value of C4 | FDR of C4 | OCA Response | Gram |
|---|---|---|---|---|---|
| Firmicutes | Streptococcus_thermophilus | 1.87E−07 | 2.30E−05 | Increase | pos |
| Actinobacteria | Bifidobacterium_breve | 4.46E−04 | 0.023 | Increase | pos |
| Firmicutes | Streptococcus_salivarius | 0.001 | 0.023 | Decrease | pos |
| Firmicutes | Lactobacillus_casei_paracasei | 0.001 | 0.03 | Increase | pos |
| Firmicutes | Lachnospiraceae_bacterium_5_ | 0.001 | 0.03 | Increase | pos |
| | 1_63FAA | | | | |
| Bacteroidetes | Alistipes_putredinis | 0.003 | 0.053 | Decrease | neg |
| Firmicutes | Lactococcus_lactis | 0.01 | 0.172 | Increase | pos |
| Bacteroidetes | Bacteroidales_bacterium_ph8 | 0.022 | 0.316 | Decrease | neg |
| Firmicutes | Subdoligranulum_unclassified | 0.024 | 0.316 | Equivocal | pos |
| Firmicutes | Dorea_longicatena | 0.026 | 0.316 | Increase | pos |
| Actinobacteria | Bifidobacterium_longum | 0.03 | 0.316 | Increase | pos |
| Firmicutes | Dialister_invisus | 0.031 | 0.316 | Decrease | neg |
| Bacteroidetes | Bacteroides_plebeius | 0.037 | 0.347 | Decrease | neg |
| Firmicutes | Ruminococcus_obeum | 0.045 | 0.389 | Decrease | pos |
| Bacteroidetes | Paraprevotella_unclassified | 0.049 | 0.389 | Decrease | neg |

Genomic Representation of Bacteria Induced by Treatment with OCA Identifies a Signature Dominated by Streptococcus thermophilus and Lactococcus lactis Consistent with Bacterial Proliferation.

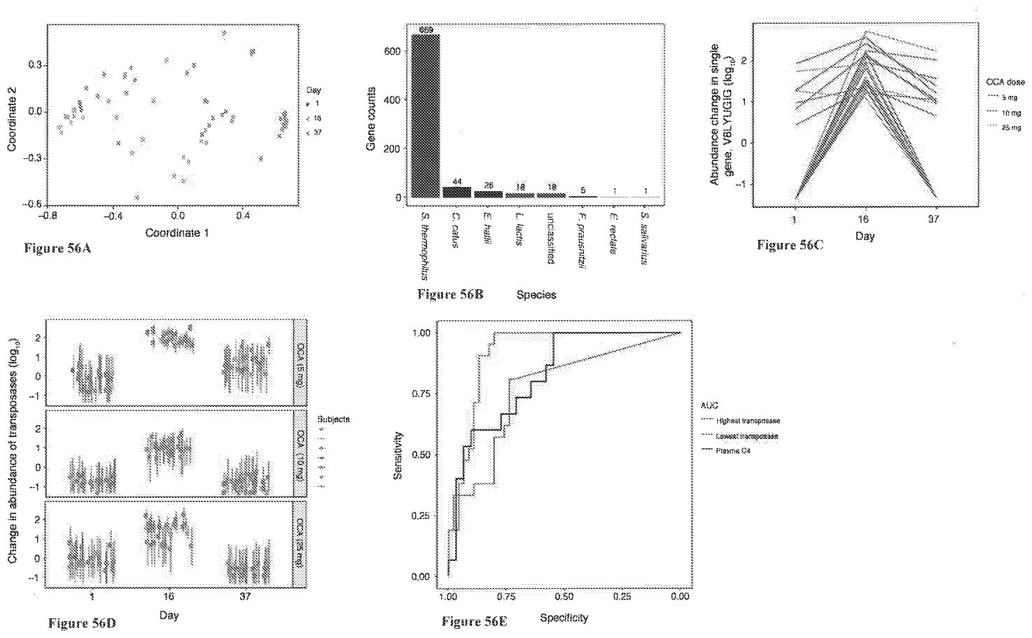

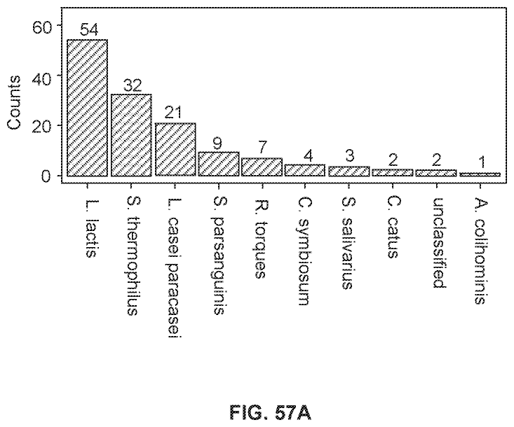

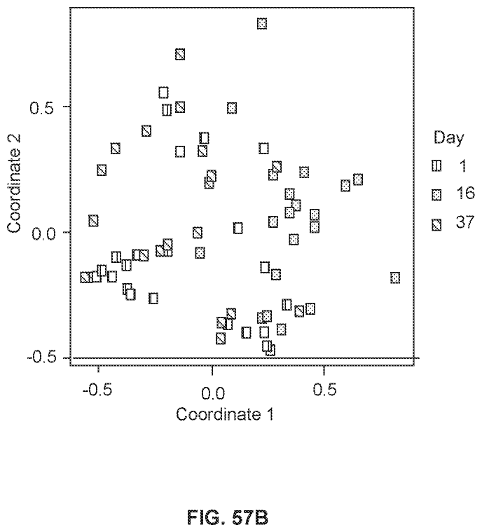

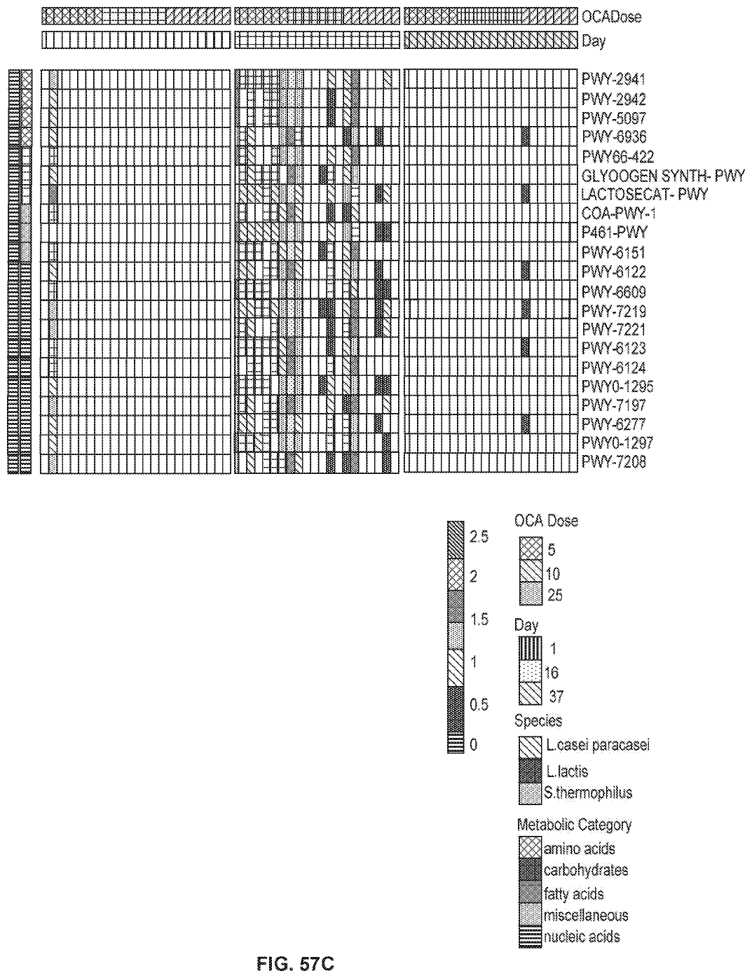

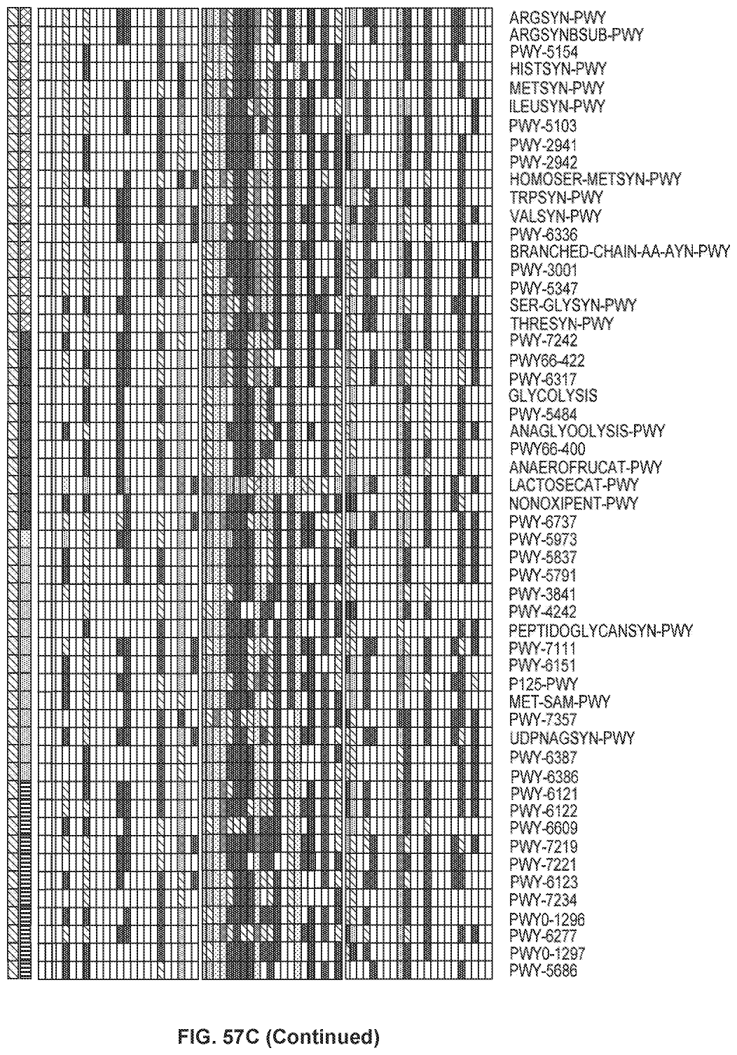

A Uniref90 high stringency genomic analysis was used to assign specific genes to the taxonomic signature of bacteria whose abundance was associated with OCA treatment and identified 782 genes assigned to eight bacterial species with a significant time-dependent effects in response to OCA treatment (FIGS. 56A and 56B). Nearly 86% of these belonged to Streptococcus thermophilus. The largest single category of genes was associated with S. thermophilus and L. lactis bacterial transposases (hypergeometric test, p=4.071×10−18) (Table 16 and FIG. 56C), enzymes that are important for the movement of mobile DNA elements throughout the genome and amongst the most abundant and ubiquitous class of genes in nature (Aziz et al., Nucleic Acids Research, 38, 4207-4217 (2010)). This family of genes showed a robust increase in representation at all three OCA doses (FIG. 56D). The abundance of transposases can be used to predict OCA treatment with higher accuracy than plasma C4 levels based on a Receiver Operating Characteristics (ROC) curve analysis (FIG. 56E). An analysis was performed to identify bacterial metabolic pathways associated with OCA treatment. A repeated measure ANOVA identified 135 MetaCyc pathways with significant time effects (FDR<0.01). The majority of the 135 pathways showing a significant association belonged to three bacterial species: Lactococcus lactis, Streptococcus thermophilus, and Lactobacillus casei/paracasei (FIG. 57A); these are Gram-positive bacteria that increased significantly with OCA treatment (Table 15). A composite view of these pathways, visualized in a multidimensional scaling plot (FIG. 57B), shows the effect of OCA and its reversibility, as the pathway abundances on Days 1 and 37 are more similar to each other compared with Day 16 (FIG. 57C). For any given species, representation of a particular gene and/or pathway is likely to simply reflect the relative abundance of that taxon in the community, so functional relevance is unclear. Association between transposases and OCA treatment is represented in FIGS. 56C-56E and Table 16).

Table 16 shows the transposases with significant (repeated measure ANOVA, FDR<0.01) time effect in response to OCA.

TABLE 16

| Gene Information | | Organism | | P Value | | |
|---|---|---|---|---|---|---|
| | | | | OCA | | |
| ProteinNames | GeneNames | Genus | Species | dose | Day | dose:Day |
| Transposase | U730_02105 | Streptococcus | Streptococcus | 0.16 | 1.4e-04 | 0.87 |
| IS1216 | | | thermophilus | | | |
| IS861, | STH8232_0423 | Streptococcus | Streptococcus | 0.47 | 1.7e-04 | 0.87 |
| transposase | | | thermophilus | | | |
| OrfB | | | | | | |
| IS861, | STH8232_1270 | Streptococcus | Streptococcus | 0.74 | 1.7e-04 | 0.87 |
| transposase | | | thermophilus | | | |
| OrfB | | | | | | |
| IS1193, | STH8232_1670 | Streptococcus | Streptococcus | 0.47 | 1.7e-04 | 0.87 |
| transposase, | | | thermophilus | | | |
| ISL3 family | | | | | | |
| Transposase | U730_306545 | Streptococcus | Streptococcus | 0.47 | 1.7e-04 | 0.87 |
| | | | thermophilus | | | |
| Transposase | STHE1630_00798 | Streptococcus | Streptococcus | 0.47 | 1.7e-04 | 0.97 |
| | | | thermophilus | | | |
| Transposase | STHE1630_01814 | Streptococcus | Streptococcus | 0.45 | 1.7e-04 | 0.88 |
| | | | thermophilus | | | |
| Transposase, | STH8232_1313 | Streptococcus | Streptococcus | 0.32 | 1.9e-04 | 0.94 |
| IS200 family | | | thermophilus | | | |
| Transposase | tnp1193 | Streptococcus | Streptococcus | 0.42 | 2.5e-04 | 0.97 |
| (IS1193) | STH8232_0914 | | thermophilus | | | |
| Transposase- | Ssal_00123 | Streptococcus | Streptococcus | 0.56 | 2.7e-04 | 0.87 |
| like protein | Ssal_01068 | | thermophilus | | | |
| | Ssal_01217 | | | | | |
| | Ssal_01551 | | | | | |
| IS1167, | STH8232_1669 | Streptococcus | Streptococcus | 0.47 | 3.5e-04 | 0.93 |
| transposase | | | thermophilus | | | |
| Transposase | LACR_C34 | Lactococcus | Lactococcus | 0.72 | 3.5e-04 | 0.91 |
| | | | lactis | | | |
| Transposase | STHE1630_00566 | Streptococcus | Streptococcus | 0.47 | 3.7e-04 | 0.87 |
| | | | thermophilus | | | |
| IS1167, | STH8232_0217 | Streptococcus | Streptococcus | 0.18 | 4.2e-04 | 0.87 |
| transposase | | | thermophilus | | | |
| Transposase for | tra905LL1204 | Streptococcus | Streptococcus | 0.19 | 4.3e-04 | 0.87 |
| insertion | L24515 | | thermophilus | | | |
| sequence | | | | | | |
| element IS905 | | | | | | |
| IS861, | STH8232_0962 | Streptococcus | Streptococcus | 0.18 | 4.5e-04 | 0.96 |
| transposase | STH8232_1450 | | thermophilus | | | |
| OrfB | | | | | | |
| IS1191, | STH8232_1033 | Streptococcus | Streptococcus | 0.33 | 5.3e-04 | 0.87 |
| transposase, | | | thermophilus | | | |
| IS256 family | | | | | | |
| IS1191, | STH8232_1031 | Streptococcus | Streptococcus | 0.16 | 6.5e-04 | 0.87 |
| transposase, | | | thermophilus | | | |
| IS256 family | | | | | | |
| Transposase | tnp-IS1193 | Streptococcus | Streptococcus | 0.47 | 6.7e-04 | 0.87 |
| | STH8232_2056 | | thermophilus | | | |
| Transposase | STHE1630_00777 | Streptococcus | Streptococcus | 0.47 | 7.0e-04 | 0.96 |
| (Fragment) | | | thermophilus | | | |
| Transposase | tnp-1 | Streptococcus | Streptococcus | 0.16 | 8.1e-04 | 0.87 |
| | STH8232_1043 | | thermophilus | | | |
| Transposase | RSSL_00033 | Streptococcus | Streptococcus | 0.16 | 9.3e-04 | 0.87 |
| | | | thermophilus | | | |
| Transposase for | tra905 LL1204 | Lactococcus | Lactococcus | 0.76 | 1.3e-03 | 0.88 |
| insertion | L24515 | | lactis | | | |
| sequence | | | | | | |
| element IS905 | | | | | | |
| IS657, | tnp657 | Streptococcus | Streptococcus | 0.47 | 1.5e-03 | 0.97 |
| transposase, | STH8232_1314 | | thermophilus | | | |
| IS200 family | | | | | | |
| Truncated | STH8232_0015 | Streptococcus | Streptococcus | 0.47 | 1.5e-03 | 0.97 |
| transposase | | | thermophilus | | | |
| Transposase | LLT7_12725 | Lactococcus | Lactococcus | 0.86 | 2.6e-03 | 0.93 |
| IS982 | | | lactis | | | |
| Transposase | #N/A | Lactococcus | Lactococcus | 0.75 | 3.2e-03 | 0.87 |
| | | | lactis | | | |
| Transposase | llh_12035 | Lactococcus | Lactococcus | 0.68 | 3.4e-03 | 0.87 |
| | | | lactis | | | |
| IS1216 | tnp | Lactococcus | Lactococcus | 0.88 | 0.01 | 0.92 |
| transposase | | | lactis | | | |
| Transposase | lilo_0827 | Lactococcus | Lactococcus | 0.78 | 0.01 | 0.92 |
| | | | lactis | | | |
| Transposase | LLT6_13245 | Lactococcus | Lactococcus | 0.71 | 0.01 | 0.89 |
| | | | lactis | | | |
| Transposase | LLT3_16435 | Lactococcus | Lactococcus | 0.81 | 0.01 | 0.95 |
| (Fragment) | | | lactis | | | |

Pathways conserved across several species in response to OCA can indicate a functional interaction. A heatmap of the statistically significant pathways for the top three bacterial taxa shows a robust time-dependent response to OCA; the associations are greatest at the lowest tested dose of OCA (FIG. 57C). Common gene pathways between these three species, as well as other taxa identified in FIG. 57A (Table 17), show that pathways associated with nucleotide and amino acid biosynthesis are enriched by OCA treatment; this is consistent with bacterial proliferation.).

Table 17 shows MetaCyc pathways identified with significant (repeated measure ANOVA, FDR<0.01) time effect in response to OCA.

TABLE 17

| | Path | dose | day | dose:day | PathID | PathName | Genus | Species |
|---|---|---|---|---|---|---|---|---|
| Amino | ARGSYN-PWY: L-arginine | 0.82 | 0.01 | 0.96 | ARGSYN- | L-arginine | Lacto- | Lactococcus_lactis |
| acids | biosynthesis I (via L- | | | | PWY | biosynthesis I | coccus | |
| | ornithine)\|g__Lactococcus.s__Lacto- | | | | | (via L- | | |
| | coccus_lactis | | | | | ornithine) | | |
| Amino | ARGSYNBSUB-PWY: | 0.85 | 0.01 | 0.95 | ARGSYN- | L-arginine | Lacto- | Lactococcus_lactis |
| acids | L-arginine biosynthesis II (acetyl | | | | BSUBPWY | biosynthesis II | coccus | |
| | cycle)\|g__Lactococcus.s__Lacto- | | | | | (acetyl cycle) | | |
| | coccus_lactis | | | | | | | |
| Amino | PWY-5154: L-arginine | 0.81 | 3.7e-03 | 0.95 | PWY- | L-arginine | Lacto- | Lactococcus_lactis |
| acids | biosynthesis III (via N-acetyl-L- | | | | 5154 | biosynthesis | coccus | |
| | citrulline)\|g__Lactococcus.s__Lacto- | | | | | III (via N- | | |
| | coccus_lactis | | | | | acetyl-L- | | |
| | | | | | | citrulline) | | |
| Amino | HISTSYN-PWY: L-histidine | 0.81 | 2.0e-03 | 0.95 | HISTSYN- | L-histidine | Lacto- | Lactococcus_lactis |
| acids | biosynthesis\|g__Lactococcus.s__Lacto- | | | | PWY | biosynthesis | coccus | |
| | coccus_lactis | | | | | | | |
| Amino | HISTSYN-PWY: L-histidine | 0.7 | 9.7e-05 | 0.98 | HISTSYN- | L-histidine | Strepto- | Streptococcus_thermo- |
| acids | biosynthesis\|g__Streptococcus.s__Strepto- | | | | PWY | biosynthesis | coccus | philus |
| | coccus_thermophilus | | | | | | | |
| Amino | HISDEG-PWY: L-histidine | 0.86 | 0.01 | 0.95 | HISDEG- | L-histidine | Strepto- | Streptococcus_par- |
| acids | degradation I\|g__Strepto- | | | | PWY | degradation I | coccus | asanguinis |
| | coccus.s__Strepto- | | | | | | | |
| | coccus_parasanguinis | | | | | | | |
| Amino | METSYN-PWY: L-homoserine | 0.81 | 3.7e-04 | 0.95 | METSYN- | L-homoserine | Lacto- | Lactococcus_lactis |
| acids | and L-methionine | | | | PWY | and L- | coccus | |
| | biosynthesis\|g__Lactococcus.s__Lacto- | | | | | methionine | | |
| | coccus_lactis | | | | | biosynthesis | | |
| Amino | ILEUSYN-PWY: L-isoleucine | 0.83 | 2.0e-03 | 0.95 | ILEUSYN- | L-isoleucine | Lacto- | Lactococcus_lactis |
| acids | biosynthesis I (from | | | | PWY | biosynthesis I | coccus | |
| | threonine)\|g__Lactococcus.s__Lacto- | | | | | (from | | |
| | coccus_lactis | | | | | threonine) | | |
| Amino | PWY-5103: L-isoleucine | 0.82 | 2.0e-03 | 0.95 | PWY- | L-isoleucine | Lacto- | Lactococcus_lactis |
| acids | biosynthesis | | | | 5103 | biosynthesis | coccus | |
| | III\|g__Lactococcus.s__Lacto- | | | | | III | | |
| | coccus_lactis | | | | | | | |
| Amino | PWY-2941: L-lysine | 0.79 | 7.1e-05 | 0.95 | PWY- | L-lysine | Lacto- | Lacto- |
| acids | biosynthesis | | | | 2941 | biosynthesis II | bacillus | bacillus_casei_para- |
| | II\|g__Lactobacillus.s__Lacto- | | | | | | | casei |
| | bacillus_casei_paracasei | | | | | | | |
| Amino | PWY-2941: L-lysine | 0.81 | 2.4e-03 | 0.96 | PWY- | L-lysine | Lacto- | Lactococcus_lactis |
| acids | biosynthesis | | | | 2941 | biosynthesis II | coccus | |
| | II\|g__Lactococcus.s__Lacto- | | | | | | | |
| | coccus_lactis | | | | | | | |
| Amino | PWY-2942: L-lysine | 0.82 | 0.01 | 0.98 | PWY- | L-lysine | Lacto- | Lacto- |
| acids | biosynthesis | | | | 2942 | biosynthesis | bacillus | bacillus_casei_para- |
| | III\|g__Lactobacillus.s__Lacto- | | | | | III | | casei |
| | bacillus_casei_paracasei | | | | | | | |
| Amino | PWY-2942: L-lysine | 0.81 | 2.9e-03 | 0.96 | PWY- | L-lysine | Lacto- | Lactococcus_lactis |
| acids | biosynthesis | | | | 2942 | biosynthesis | coccus | |
| | III\|g__Lactococcus.s__Lacto- | | | | | III | | |
| | coccus_lactis | | | | | | | |
| Amino | PWY-5097: L-lysine | 0.82 | 0.01 | 0.98 | PWY- | L-lysine | Lacto- | Lacto- |
| acids | biosynthesis | | | | 5097 | biosynthesis | bacillus | bacillus_casei_para- |
| | VI\|g__Lactobacillus.s__Lacto- | | | | | VI | | casei |
| | bacillus_casei_paracasei | | | | | | | |
| Amino | HOMOSER-METSYN-PWY: | 0.84 | 1.5e-04 | 0.95 | HOMOSER- | L-methionine | Lacto- | Lactococcus_lactis |
| acids | L-methionine biosynthesis | | | | METSYN- | biosynthesis I | coccus | |
| | I\|g__Lactococcus.s__Lacto- | | | | PWY | | | |
| | coccus_lactis | | | | | | | |
| Amino | TRPSYN-PWY: L-tryptophan | 0.82 | 0.01 | 0.95 | TRPSYN- | L-tryptophan | Lacto- | Lactococcus_lactis |
| acids | biosynthesis\|g__Lactococcus.s__Lacto- | | | | PWY | biosynthesis | coccus | |
| | coccus_lactis | | | | | | | |
| Amino | VALSYN-PWY: L-valine | 0.85 | 1.6e-03 | 0.96 | VALSYN- | L-valine | Lacto- | Lactococcus_lactis |
| acids | biosynthesis\|g__Lactococcus.s__Lacto- | | | | PWY | biosynthesis | coccus | |
| | coccus_lactis | | | | | | | |
| Amino | VALSYN-PWY: L-valine | 0.41 | 9.7e-05 | 0.95 | VALSYN- | L-valine | Strepto- | Streptococcus_thermo- |
| acids | biosynthesis\|g__Streptococcus.s__Strepto- | | | | PWY | biosynthesis | coccus | philus |
| | coccus_thermophilus | | | | | | | |
| Amino | PWY-6936: seleno-amino acid | 0.85 | 0.01 | 0.98 | PWY- | seleno-amino | Lacto- | Lacto- |
| acids | biosynthesis\|g__Lactobacillus.s__Lacto- | | | | 6936 | acid | bacillus | bacillus_casei_para- |
| | bacillus_casei_paracasei | | | | | biosynthesis | | casei |
| Amino | PWY-6936: seleno-amino acid | 0.81 | 2.4e-03 | 0.95 | PWY- | seleno-amino | Lacto- | Lactococcus_lactis |
| acids | biosynthesis\|g__Lactococcus.s__Lacto- | | | | 6936 | acid | coccus | |
| | coccus_lactis | | | | | biosynthesis | | |
| Amino | COMPLETE-ARO-PWY: | 0.85 | 0.01 | 0.95 | COMPLETE- | superpathway | Clostri- | Clostridium_symbiosum |
| acids | superpathway of aromatic amino | | | | AROPWY | of aromatic | dium | |
| | acid | | | | | amino acid | | |
| | biosynthesis\|g__Clostridium.s__Clostrid- | | | | | biosynthesis | | |
| | ium_symbiosum | | | | | | | |
| Amino | COMPLETE-ARO-PWY: | 0.41 | 1.3e-04 | 0.98 | COMPLETE- | superpathway | Strepto- | Streptococcus_thermo- |
| acids | superpathway of aromatic amino | | | | AROPWY | of aromatic | coccus | philus |
| | acid | | | | | amino acid | | |
| | biosynthesis\|g__Streptococcus.s__Strepto- | | | | | biosynthesis | | |
| | coccus_thermophilus | | | | | | | |
| Amino | BRANCHED-CHAIN-AA- | 0.82 | 2.0e-03 | 0.95 | BRANCHED- | superpathway | Lacto- | Lactococcus_lactis |
| acids | SYN-PWY: superpathway of | | | | CHAIN- | of branched | coccus | |
| | branched amino acid | | | | AA- | amino acid | | |
| | biosynthesis\|g__Lactococcus.s__Lacto- | | | | SYN- | biosynthesis | | |
| | coccus_lactis | | | | PWY | | | |
| Amino | PWY-3001: superpathway of L- | 0.79 | 0.01 | 0.95 | PWY- | superpathway | Lacto- | Lactococcus_lactis |
| acids | isoleucine biosynthesis | | | | 3001 | of L- | coccus | |
| | I\|g__Lactococcus.s__Lacto- | | | | | isoleucine | | |
| | coccus_lactis | | | | | biosynthesis I | | |
| Amino | PWY-5347: superpathway of L- | 0.81 | 3.3e-04 | 0.95 | PWY- | superpathway | Lacto- | Lactococcus_lactis |
| acids | methionine biosynthesis | | | | 5347 | of L- | coccus | |
| | (transsulfuration)\|g__Lacto- | | | | | methionine | | |
| | coccus.s__Lactococcus_lactis | | | | | biosynthesis | | |
| | | | | | | (transsulfura- | | |
| | | | | | | tion) | | |
| Amino | SER-GLYSYN-PWY: | 0.85 | 3.0e-03 | 0.96 | SER- | superpathway | Lacto- | Lactococcus_lactis |
| acids | superpathway of L-serine and | | | | GLYSYN- | of L-serine | coccus | |
| | glycine biosynthesis | | | | PWY | and glycine | | |
| | I\|g__Lactococcus.s__Lacto- | | | | | biosynthesis I | | |
| | coccus_lactis | | | | | | | |
| Amino | THRESYN-PWY: superpathway | 0.81 | 1.9e-03 | 0.95 | THRESYN- | superpathway | Lacto- | Lactococcus_lactis |
| acids | of L-threonine | | | | PWY | of L-threonine | coccus | |
| | biosynthesis\|g__Lactococcus.s__Lacto- | | | | | biosynthesis | | |
| | coccus_lactis | | | | | | | |
| nucleic | PWY-6121: 5-aminoimidazole | 0.84 | 2.9e-03 | 0.96 | PWY- | 5-amino- | Lacto- | Lactococcus_lactis |
| acid | ribonucleotide biosynthesis | | | | 6121 | imidazole | coccus | |
| | I\|g__Lactococcus.s__Lacto- | | | | | ribonucleotide | | |
| | coccus_lactis | | | | | biosynthesis I | | |
| nucleic | PWY-6121: 5-aminoimidazole | 0.7 | 1.4e-04 | 0.96 | PWY- | 5-amino- | Strepto- | Streptococcus_thermo- |
| acid | ribonucleotide biosynthesis | | | | 6121 | imidazole | coccus | philus |
| | I\|g__Streptococcus.s__Strepto- | | | | | ribonucleotide | | |
| | coccus_thermophilus | | | | | biosynthesis I | | |
| nucleic | PWY-6122: 5-aminoimidazole | 0.84 | 2.8e-04 | 0.95 | PWY- | 5-amino- | Lacto- | Lacto- |
| acid | ribonucleotide biosynthesis | | | | 6122 | imidazole | bacillus | bacillus_casei_para- |
| | II\|g__Lactobacillus.s__Lacto- | | | | | ribonucleotide | | casei |
| | bacillus_casei_paracasei | | | | | biosynthesis II | | |
| nucleic | PWY-6122: 5-aminoimidazole | 0.85 | 1.7e-03 | 0.98 | PWY- | 5-amino- | Lacto- | Lactococcus_lactis |
| acid | ribonucleotide biosynthesis | | | | 6122 | imidazole | coccus | |
| | II\|g__Lactococcus.s__Lacto- | | | | | ribonucleotide | | |
| | coccus_lactis | | | | | biosynthesis II | | |
| nucleic | PWY-6122: 5-aminoimidazole | 0.41 | 1.2e-04 | 0.95 | PWY- | 5-amino- | Strepto- | Streptococcus_thermo- |
| acid | ribonucleotide biosynthesis | | | | 6122 | imidazole | coccus | philus |
| | II\|g__Streptococcus.s__Strepto- | | | | | ribonucleotide | | |
| | coccus_thermophilus | | | | | biosynthesis II | | |
| nucleic | PWY-6609: adenine and | 0.82 | 1.2e-04 | 0.98 | PWY- | adenine and | Lacto- | Lacto- |
| acid | adenosine salvage | | | | 6609 | adenosine | bacillus | bacillus_casei_para- |
| | III\|g__Lactobacillus.s__Lacto- | | | | | salvage III | | casei |
| | bacillus_casei_paracasei | | | | | | | |
| nucleic | PWY-6609: adenine and | 0.73 | 1.6e-03 | 0.95 | PWY- | adenine and | Lacto- | Lactococcus_lactis |
| acid | adenosine salvage | | | | 6609 | adenosine | coccus | |
| | III\|g__Lactococcus.s__Lacto- | | | | | salvage III | | |
| | coccus_lactis | | | | | | | |
| nucleic | PWY-6609: adenine and | 0.9 | 0.01 | 0.96 | PWY- | adenine and | Strepto- | Streptococcus_para- |
| acid | adenosine salvage | | | | 6609 | adenosine | coccus | sanguinis |
| | III\|g__Streptococcus.s__Strepto- | | | | | salvage III | | |
| | coccus_parasanguinis | | | | | | | |
| nucleic | PWY-6609: adenine and | 0.7 | 7.1e-05 | 0.95 | PWY- | adenine and | Strepto- | Streptococcus_thermo- |
| acid | adenosine salvage | | | | 6609 | adenosine | coccus | philus |
| | III\|g__Streptococcus.s__Strepto- | | | | | salvage III | | |
| | coccus_thermophilus | | | | | | | |
| nucleic | PWY-7220: adenosine | 0.7 | 1.4e-04 | 0.95 | PWY- | adenosine | Strepto- | Streptococcus_thermo- |
| acid | deoxyribonucleotides de novo | | | | 7220 | deoxyribo- | coccus | philus |
| | biosynthesis | | | | | nucleotides | | |
| | II\|g__Streptococcus.s__Strepto- | | | | | de novo | | |
| | coccus_thermophilus | | | | | biosynthesis II | | |
| nucleic | PWY-7219: adenosine | 0.81 | 4.3e-05 | 0.95 | PWY- | adenosine | Lacto- | Lacto- |
| acid | ribonucleotides de novo | | | | 7219 | ribonucleotides | bacillus | bacillus_casei_para- |
| | biosynthesis\|g__Lactobacillus.s__Lacto- | | | | | de novo | | casei |
| | bacillus_casei_paracasei | | | | | biosynthesis | | |
| nucleic | PWY-7219: adenosine | 0.84 | 0.01 | 0.96 | PWY- | adenosine | Lacto- | Lactococcus_lactis |
| acid | ribonucleotides de novo | | | | 7219 | ribonucleotides | coccus | |
| | biosynthesis\|g__Lactococcus.s__Lacto- | | | | | de novo | | |
| | coccus_lactis | | | | | biosynthesis | | |
| nucleic | PWY-7219: adenosine | 0.88 | 3.3e-03 | 0.95 | PWY- | adenosine | Strepto- | Streptococcus_para- |
| acid | ribonucleotides de novo | | | | 7219 | ribonucleotides | coccus | sanguinis |
| | biosynthesis\|g__Streptococcus.s__Strepto- | | | | | de novo | | |
| | coccus_parasanguinis | | | | | biosynthesis | | |
| nucleic | PWY-7219: adenosine | 0.73 | 2.7e-03 | 0.95 | PWY- | adenosine | Strepto- | Streptococcus_sali- |
| acid | ribonucleotides de novo | | | | 7219 | ribonucleotides | coccus | varius |
| | biosynthesis\|g__Streptococcus.s__Strepto- | | | | | de novo | | |
| | coccus_salivarius | | | | | biosynthesis | | |
| nucleic | PWY-7219: adenosine | 0.41 | 9.9e-05 | 0.95 | PWY- | adenosine | Strepto- | Streptococcus_thermo- |
| acid | ribonucleotides de novo | | | | 7219 | ribonucleotides | coccus | philus |
| | biosynthesis\|g__Streptococcus.s__Strepto- | | | | | de novo | | |
| | coccus_thermophilus | | | | | biosynthesis | | |
| nucleic | PWY-7222: guanosine | 0.7 | 1.4e-04 | 0.95 | PWY- | guanosine | Strepto- | Streptococcus_thermo- |
| acid | deoxyribonucleotides de novo | | | | 7222 | deoxyribo- | coccus | philus |
| | biosynthesis | | | | | nucleotides | | |
| | II\|g__Streptococcus.s__Strepto- | | | | | de novo | | |
| | coccus_thermophilus | | | | | biosynthesis II | | |
| nucleic | PWY-7221: guanosine | 0.85 | 3.8e-03 | 0.95 | PWY- | guanosine | Clostrid- | Clostridium_symbiosum |
| acid | ribonucleotides de novo | | | | 7221 | ribonucleotides | ium | |
| | biosynthesis\|g__Clostridium.s__Clostrid- | | | | | de novo | | |
| | ium_symbiosum | | | | | biosynthesis | | |
| nucleic | PWY-7221: guanosine | 0.81 | 3.5e-04 | 0.96 | PWY- | guanosine | Lacto- | Lacto- |
| acid | ribonucleotides de novo | | | | 7221 | ribonucleotides | bacillus | bacillus_casei_para- |
| | biosynthesis\|g__Lactobacillus.s__Lacto- | | | | | de novo | | casei |
| | bacillus_casei_paracasei | | | | | biosynthesis | | |
| nucleic | PWY-7221: guanosine | 0.83 | 0.01 | 0.95 | PWY- | guanosine | Lacto- | Lactococcus_lactis |
| acid | ribonucleotides de novo | | | | 7221 | ribonucleotides | coccus | |
| | biosynthesis\|g__Lactococcus.s__Lacto- | | | | | de novo | | |
| | coccus_lactis | | | | | biosynthesis | | |
| nucleic | PWY-7221: guanosine | 0.41 | 1.1e-04 | 0.95 | PWY- | guanosine | Strepto- | Streptococcus_thermo- |
| acid | ribonucleotides de novo | | | | 7221 | ribonucleotides | coccus | philus |
| | biosynthesis\|g__Streptococcus.s__Strepto- | | | | | de novo | | |
| | coccus_thermophilus | | | | | biosynthesis | | |
| nucleic | PWY-6123: inosine-5′- | 0.79 | 1.3e-04 | 0.95 | PWY- | inosine-5′- | Lacto- | Lacto- |
| acid | phosphate biosynthesis | | | | 6123 | phosphate | bacillus | bacillus_casei_para- |
| | I\|g__Lactobacillus.s__Lacto- | | | | | biosynthesis I | | casei |
| | bacillus_casei_paracasei | | | | | | | |
| nucleic | PWY-6123: inosine-5′- | 0.79 | 0.01 | 0.95 | PWY- | inosine-5′- | Lacto- | Lactococcus_lactis |
| acid | phosphate biosynthesis | | | | 6123 | phosphate | coccus | |
| | I\|g__Lactococcus.s__Lacto- | | | | | biosynthesis I | | |
| | coccus_lactis | | | | | | | |
| nucleic | PWY-6123: inosine-5′- | 0.94 | 0.01 | 0.95 | PWY- | inosine-5′- | Strepto- | Streptococcus_para- |
| acid | phosphate biosynthesis | | | | 6123 | phosphate | coccus | sanguinis |
| | I\|g__Streptococcus.s__Strepto- | | | | | biosynthesis I | | |
| | coccus_parasanguinis | | | | | | | |
| nucleic | PWY-6123: inosine-5′- | 0.81 | 0.01 | 0.95 | PWY- | inosine-5′- | Strepto- | Streptococcus_sali- |
| acid | phosphate biosynthesis | | | | 6123 | phosphate | coccus | varius |
| | I\|g__Streptococcus.s__Strepto- | | | | | biosynthesis I | | |
| | coccus_salivarius | | | | | | | |
| nucleic | PWY-6123: inosine-5′- | 0.7 | 7.1e-05 | 0.98 | PWY- | inosine-5′- | Strepto- | Streptococcus_thermo- |
| acid | phosphate biosynthesis | | | | 6123 | phosphate | coccus | philus |
| | I\|g__Streptococcus.s__Strepto- | | | | | biosynthesis I | | |
| | coccus_thermophilus | | | | | | | |
| nucleic | PWY-6124: inosine-5′- | 0.82 | 4.8e-03 | 0.98 | PWY- | inosine-5′- | Lacto- | Lacto- |
| acid | phosphate biosynthesis | | | | 6124 | phosphate | bacillus | bacillus_casei_para- |
| | II\|g__Lactobacillus.s__Lacto- | | | | | biosynthesis II | | casei |
| | bacillus_casei_paracasei | | | | | | | |
| nucleic | PWY-6124: inosine-5′- | 0.94 | 0.01 | 0.95 | PWY- | inosine-5′- | Strepto- | Streptococcus_para- |
| acid | phosphate biosynthesis | | | | 6124 | phosphate | coccus | sanguinis |
| | II\|g__Streptococcus.s__Strepto- | | | | | biosynthesis II | | |
| | coccus_parasanguinis | | | | | | | |
| nucleic | PWY-6124: inosine-5′- | 0.79 | 2.6e-03 | 0.95 | PWY- | inosine-5′- | Strepto- | Streptococcus_sali- |
| acid | phosphate biosynthesis | | | | 6124 | phosphate | coccus | varius |
| | II\|g__Streptococcus.s__Strepto- | | | | | biosynthesis II | | |
| | coccus_salivarius | | | | | | | |
| nucleic | PWY-6124: inosine-5′- | 0.41 | 7.1e-05 | 0.95 | PWY- | inosine-5′- | Strepto- | Streptococcus_thermo- |
| acid | phosphate biosynthesis | | | | 6124 | phosphate | coccus | philus |
| | II\|g__Streptococcus.s__Strepto- | | | | | biosynthesis II | | |
| | coccus_thermophilus | | | | | | | |
| nucleic | PWY-7234: inosine-5′- | 0.79 | 2.0e-03 | 0.95 | PWY- | inosine-5′- | Lacto- | Lactococcus_lactis |
| acid | phosphate biosynthesis | | | | 7234 | phosphate | coccus | |
| | III\|g__Lactococcus.s__Lacto- | | | | | biosynthesis | | |
| | coccus_lactis | | | | | III | | |
| nucleic | PWY-7234: inosine-5′- | 0.94 | 0.01 | 0.95 | PWY- | inosine-5′- | Strepto- | Streptococcus_para- |
| acid | phosphate biosynthesis | | | | 7234 | phosphate | coccus | sanguinis |
| | III\|g__Streptococcus.s__Strepto- | | | | | biosynthesis | | |
| | coccus_parasanguinis | | | | | III | | |
| nucleic | PWY0-1296: purine | 0.84 | 7.1e-05 | 0.98 | PWY0- | purine | Lacto- | Lacto- |
| acid | ribonucleosides | | | | 1296 | ribonucleosides | bacillus | bacillus_casei_para- |
| | degradation\|g__Lactobacillus.s__Lacto- | | | | | degradation | | casei |
| | bacillus_casei_paracasei | | | | | | | |
| nucleic | PWY0-1296: purine | 0.78 | 1.7e-03 | 0.95 | PWY0- | purine | Lacto- | Lactococcus_lactis |
| acid | ribonucleosides | | | | 1296 | ribonucleosides | coccus | |
| | degradation\|g__Lactococcus.s__Lacto- | | | | | degradation | | |
| | coccus_lactis | | | | | | | |
| nucleic | PWY0-1296: purine | 0.7 | 7.1e-05 | 0.95 | PWY0- | purine | Strepto- | Streptococcus_thermo- |
| acid | ribonucleosides | | | | 1296 | ribonucleosides | coccus | philus |
| | degradation\|g__Streptococcus.s__Strepto- | | | | | degradation | | |
| | coccus_thermophilus | | | | | | | |
| nucleic | PWY-7199: pyrimidine | 0.83 | 1.1e-03 | 0.95 | PWY- | pyrimidine | Strepto- | Streptococcus_thermo- |
| acid | deoxyribonucleosides | | | | 7199 | deoxyribo- | coccus | philus |
| | salvage\|g__Streptococcus.s__Strepto- | | | | | nucleosides | | |
| | coccus_thermophilus | | | | | salvage | | |
| nucleic | PWY-7197: pyrimidine | 0.81 | 2.7e-04 | 0.97 | PWY- | pyrimidine | Lacto- | Lacto- |
| acid | deoxyribonucleotide | | | | 7197 | deoxyribo- | bacillus | bacillus_casei_para- |
| | phosphorylation\|g__Lacto- | | | | | nucleotide | | casei |
| | bacillus.s__Lacto- | | | | | phosphorylation | | |
| | bacillus_casei_paracasei | | | | | | | |
| nucleic | PWY-7197: pyrimidine | 0.7 | 7.1e-05 | 0.96 | PWY- | pyrimidine | Strepto- | Streptococcus_thermo- |
| acid | deoxyribonucleotide | | | | 7197 | deoxyribo- | coccus | philus |
| | phosphorylation\|g__Strepto- | | | | | nucleotide | | |
| | coccus.s__Strepto- | | | | | phosphorylation | | |
| | coccus_thermophilus | | | | | | | |
| nucleic | PWY-6277: superpathway of 5- | 0.84 | 2.8e-04 | 0.95 | PWY- | superpathway | Lacto- | Lacto- |
| acid | aminoimidazole ribonucleotide | | | | 6277 | of 5-amino- | bacillus | bacillus_casei_para- |
| | biosynthesis\|g__Lactobacillus.s__Lacto- | | | | | imidazole | | casei |
| | bacillus_casei_paracasei | | | | | ribonucleotide | | |
| | | | | | | biosynthesis | | |
| nucleic | PWY-6277: superpathway of 5- | 0.85 | 1.7e-03 | 0.98 | PWY- | superpathway | Lacto- | Lactococcus_lactis |
| acid | aminoimidazole ribonucleotide | | | | 6277 | of 5-amino- | coccus | |
| | biosynthesis\|g__Lactococcus.s__Lacto- | | | | | imidazole | | |
| | coccus_lactis | | | | | ribonucleotide | | |
| | | | | | | biosynthesis | | |
| nucleic | PWY-6277: superpathway of 5- | 0.41 | 1.2e-04 | 0.95 | PWY- | superpathway | Strepto- | Streptococcus_thermo- |
| acid | aminoimidazole ribonucleotide | | | | 6277 | of 5-amino- | coccus | philus |
| | biosynthesis\|g__Streptococcus.s__Strepto- | | | | | imidazole | | |
| | coccus_thermophilus | | | | | ribonucleotide | | |
| | | | | | | biosynthesis | | |
| nucleic | PWY-7229: superpathway of | 0.73 | 7.1e-05 | 0.96 | PWY- | superpathway | Strepto- | Streptococcus_thermo- |
| acid | adenosine nucleotides de novo | | | | 7229 | of adenosine | coccus | philus |
| | biosynthesis | | | | | nucleotides de | | |
| | I\|g__Streptococcus.s__Strepto- | | | | | novo | | |
| | coccus_thermophilus | | | | | biosynthesis I | | |
| nucleic | PWY-6126: superpathway of | 0.7 | 1.4e-04 | 0.95 | PWY- | superpathway | Strepto- | Streptococcus_thermo- |
| acid | adenosine nucleotides de novo | | | | 6126 | of adenosine | coccus | philus |
| | biosynthesis | | | | | nucleotides de | | |
| | II\|g__Streptococcus.s__Strepto- | | | | | novo | | |
| | coccus_thermophilus | | | | | biosynthesis II | | |
| nucleic | PWY-7228: superpathway of | 0.7 | 9.7e-05 | 0.95 | PWY- | superpathway | Strepto- | Streptococcus_thermo- |
| acid | guanosine nucleotides de novo | | | | 7228 | of guanosine | coccus | philus |
| | biosynthesis | | | | | nucleotides de | | |
| | I\|g__Streptococcus.s__Strepto- | | | | | novo | | |
| | coccus_thermophilus | | | | | biosynthesis I | | |
| nucleic | PWY-6125: superpathway of | 0.7 | 1.4e-04 | 0.95 | PWY- | superpathway | Strepto- | Streptococcus_thermo- |
| acid | guanosine nucleotides de novo | | | | 6125 | of guanosine | coccus | philus |
| | biosynthesis | | | | | nucleotides de | | |
| | II\|g__Streptococcus.s__Strepto- | | | | | novo | | |
| | coccus_thermophilus | | | | | biosynthesis II | | |
| nucleic | PWY0-1297: superpathway of | 0.82 | 2.8e-04 | 0.97 | PWY0- | superpathway | Lacto- | Lacto- |
| acid | purine deoxyribonucleosides | | | | 1297 | of purine | bacillus | bacillus_casei_para- |
| | degradation\|g__Lactobacillus.s__Lacto- | | | | | deoxyribo- | | casei |
| | bacillus_casei_paracasei | | | | | nucleosides | | |
| | | | | | | degradation | | |
| nucleic | PWY0-1297: superpathway of | 0.7 | 0.01 | 0.95 | PWY0- | superpathway | Lacto- | Lactococcus_lactis |
| acid | purine deoxyribonucleosides | | | | 1297 | of purine | coccus | |
| | degradation\|g__Lactococcus.s__Lacto- | | | | | deoxyribo- | | |
| | coccus_lactis | | | | | nucleosides | | |
| | | | | | | degradation | | |
| nucleic | PWY66-409: superpathway of | 0.41 | 2.1e-04 | 0.95 | PWY66- | superpathway | Strepto- | Streptococcus_thermo- |
| acid | purine nucleotide | | | | 409 | of purine | coccus | philus |
| | salvage\|g__Streptococcus.s__Strepto- | | | | | nucleotide | | |
| | coccus_thermophilus | | | | | salvage | | |
| nucleic | PWY-7208: superpathway of | 0.82 | 3.1e-03 | 0.98 | PWY- | superpathway | Lacto- | Lacto- |
| acid | pyrimidine nucleobases | | | | 7208 | of pyrimidine | bacillus | bacillus_casei_para- |
| | salvage\|g__Lactobacillus.s__Lacto- | | | | | nucleobases | | casei |
| | bacillus_casei_paracasei | | | | | salvage | | |
| nucleic | PWY-7208: superpathway of | 0.82 | 0.01 | 0.95 | PWY- | superpathway | Blautia | Ruminococcus_torques |
| acid | pyrimidine nucleobases | | | | 7208 | of pyrimidine | | |
| | salvage\|g__Blautia.s__Rumino- | | | | | nucleobases | | |
| | coccus_torques | | | | | salvage | | |
| nucleic | PWY-7208: superpathway of | 0.7 | 9.7e-05 | 0.96 | PWY- | superpathway | Strepto- | Streptococcus_thermo- |
| acid | pyrimidine nucleobases | | | | 7208 | of pyrimidine | coccus | philus |
| | salvage\|g__Streptococcus.s__Strepto- | | | | | nucleobases | | |
| | coccus_thermophilus | | | | | salvage | | |
| nucleic | PWY-5686: UMP | 0.81 | 5.8e-04 | 0.95 | PWY- | UMP | Lacto- | Lactococcus_lactis |
| acid | biosynthesis\|g__Lactococcus.s__Lacto- | | | | 5686 | biosynthesis | coccus | |
| | coccus_lactis | | | | | | | |
| nucleic | PWY-7242: D-fructuronate | 0.89 | 5.2e-04 | 0.95 | PWY- | D-fructuronate | Lacto- | Lactococcus_lactis |
| acid | degradation\|g__Lactococcus.s__Lacto- | | | | 7242 | degradation | coccus | |
| | coccus_lactis | | | | | | | |
| nucleic | PWY66-422: D-galactose | 0.85 | 2.9e-03 | 0.98 | PWY66- | D-galactose | Lacto- | Lacto- |
| acid | degradation V (Leloir | | | | 422 | degradation V | bacillus | bacillus_casei_para- |
| | pathway)\|g__Lactobacillus.s__Lacto- | | | | | (Leloir | | casei |
| | bacillus_casei_paracasei | | | | | pathway) | | |
| nucleic | PWY66-422: D-galactose | 0.84 | 0.01 | 0.96 | PWY66- | D-galactose | Lacto- | Lactococcus_lactis |
| acid | degradation V (Leloir | | | | 422 | degradation V | coccus | |
| | pathway)\|g__Lactococcus.s__Lacto- | | | | | (Leloir | | |
| | coccus_lactis | | | | | pathway) | | |
| nucleic | PWY66-422: D-galactose | 0.41 | 3.8e-04 | 0.98 | PWY66- | D-galactose | Strepto- | Streptococcus_thermo- |
| acid | degradation V (Leloir | | | | 422 | degradation V | coccus | philus |
| | pathway)\|g__Streptococcus.s__Strepto- | | | | | (Leloir | | |
| | coccus_thermophilus | | | | | pathway) | | |
| nucleic | PWY66-422: D-galactose | 0.81 | 2.9e-03 | 0.95 | PWY66- | D-galactose | unclassi- | unclassified |
| acid | degradation V (Leloir | | | | 422 | degradation V | fied | |
| | pathway)\|unclassified | | | | | (Leloir | | |
| | | | | | | pathway) | | |
| nucleic | PWY-6317: galactose | 0.85 | 1.7e-03 | 0.96 | PWY- | galactose | Lacto- | Lactococcus_lactis |
| acid | degradation I (Leloir | | | | 6317 | degradation I | coccus | |
| | pathway)\|g__Lactococcus.s__Lacto- | | | | | (Leloir | | |
| | coccus_lactis | | | | | pathway) | | |
| nucleic | PWY-6317: galactose | 0.41 | 3.8e-04 | 0.98 | PWY- | galactose | Strepto- | Streptococcus_thermo- |
| acid | degradation I (Leloir | | | | 6317 | degradation I | coccus | philus |
| | pathway)\|g__Streptococcus.s__Strepto- | | | | | (Leloir | | |
| | coccus_thermophilus | | | | | pathway) | | |
| nucleic | GLYCOGENSYNTH-PWY: | 0.73 | 9.5e-05 | 0.95 | GLYCOGEN- | glycogen | Lacto- | Lacto- |
| acid | glycogen biosynthesis I (from | | | | SYNTHPWY | biosynthesis I | bacillus | bacillus_casei_para- |
| | ADP-D- | | | | | (from ADP-D- | | casei |
| | Glucose)\|g__Lactobacillus.s__Lacto- | | | | | Glucose) | | |
| | bacillus_casei_paracasei | | | | | | | |
| nucleic | GLYCOGENSYNTH-PWY: | 0.81 | 2.4e-03 | 0.96 | GLYCOGEN- | glycogen | unclassi- | unclassified |
| acid | glycogen biosynthesis I (from | | | | SYNTHPWY | biosynthesis I | fied | |
| | ADP-D-Glucose)\|unclassified | | | | | (from ADP-D- | | |
| | | | | | | Glucose) | | |
| nucleic | GLYCOLYSIS: glycolysis I | 0.91 | 0.01 | 0.95 | GLYCOLY- | glycolysis I | Lacto- | Lactococcus_lactis |
| acid | (from glucose 6- | | | | SIS | (from glucose | coccus | |
| | phosphate)\|g__Lactococcus.s__Lacto- | | | | | 6-phosphate) | | |
| | coccus_lactis | | | | | | | |
| nucleic | PWY-5484: glycolysis II (from | 0.92 | 0.01 | 0.95 | PWY- | glycolysis II | Lacto- | Lactococcus_lactis |
| acid | fructose 6- | | | | 5484 | (from fructose | coccus | |
| | phosphate)\|g__Lactococcus.s__Lacto- | | | | | 6-phosphate) | | |
| | coccus_lactis | | | | | | | |
| nucleic | ANAGLYCOLYSIS-PWY: | 0.85 | 1.6e-03 | 0.95 | ANAGLY- | glycolysis III | Lacto- | Lactococcus_lactis |
| acid | glycolysis III (from | | | | COLY- | (from glucose) | coccus | |
| | glucose)\|g__Lactococcus.s__Lacto- | | | | SISPWY | | | |
| | coccus_lactis | | | | | | | |
| nucleic | PWY66-400: glycolysis VI | 0.9 | 0.01 | 0.95 | PWY66- | glycolysis VI | Lacto- | Lactococcus_lactis |
| acid | (metazoan)\|g__Lactococcus.s__Lacto- | | | | 400 | (metazoan) | coccus | |
| | coccus_lactis | | | | | | | |
| nucleic | ANAEROFRUCAT-PWY: | 0.91 | 0.01 | 0.95 | ANAERO- | homolactic | Lacto- | Lactococcus_lactis |
| acid | homolactic | | | | FRUCAT- | fermentation | coccus | |
| | fermentation\|g__Lactococcus.s__Lacto- | | | | PWY | | | |
| | coccus_lactis | | | | | | | |
| nucleic | RHAMCAT-PWY: L-rhamnose | 0.82 | 0.01 | 0.95 | RHAMCAT- | L-rhamnose | Blautia | Ruminococcus_torques |
| acid | degradation | | | | PWY | degradation I | | |
| | I\|g__Blautia.s__Ruminococcus_torques | | | | | | | |
| nucleic | LACTOSECAT-PWY: lactose | 0.81 | 9.5e-05 | 0.95 | LACTOSE- | lactose and | Lacto- | Lacto- |
| acid | and galactose degradation | | | | CATPWY | galactose | bacillus | bacillus_casei_para- |
| | I\|g__Lactobacillus.s__Lacto- | | | | | degradation I | | casei |
| | bacillus_casei_paracasei | | | | | | | |
| nucleic | LACTOSECAT-PWY: lactose | 0.85 | 2.4e-03 | 0.96 | LACTOSE- | lactose and | Lacto- | Lactococcus_lactis |
| acid | and galactose degradation | | | | CATPWY | galactose | coccus | |
| | I\|g__Lactococcus.s__Lacto- | | | | | degradation I | | |
| | coccus_lactis | | | | | | | |
| nucleic | NONOXIPENT-PWY: pentose | 0.81 | 0.01 | 0.95 | NONOXI- | pentose | Lacto- | Lactococcus_lactis |
| acid | phosphate pathway (non- | | | | PENTPWY | phosphate | coccus | |
| | oxidative | | | | | pathway (non- | | |
| | branch)\|g__Lactococcus.s__Lacto- | | | | | oxidative | | |
| | coccus_lactis | | | | | branch) | | |
| nucleic | PWY-6737: starch degradation | 0.91 | 1.6e-03 | 0.96 | PWY- | starch | Lacto- | Lactococcus_lactis |
| acid | V\|g__Lactococcus.s__Lacto- | | | | 6737 | degradation V | coccus | |
| | coccus_lactis | | | | | | | |
| nucleic | PWY-621: sucrose degradation | 0.83 | 0.01 | 0.97 | PWY- | sucrose | Strepto- | Streptococcus_para- |
| acid | III (sucrose | | | | 621 | degradation | coccus | sanguinis |
| | invertase)\|g__Streptococcus.s__Strepto- | | | | | III (sucrose | | |
| | coccus_parasanguinis | | | | | invertase) | | |
| fatty | PWY-5973: cis-vaccenate | 0.87 | 3.2e-03 | 0.96 | PWY- | cis-vaccenate | Lacto- | Lactococcus_lactis |
| acids | biosynthesis\|g__Lactococcus.s__Lacto- | | | | 5973 | biosynthesis | coccus | |
| | coccus_lactis | | | | | | | |
| fatty | PWY-5973: cis-vaccenate | 0.83 | 2.9e-03 | 0.95 | PWY- | cis-vaccenate | Blautia | Ruminococcus_torques |
| acids | biosynthesis\|g__Blautia.s__Rumino- | | | | 5973 | biosynthesis | | |
| | coccus_torques | | | | | | | |
| fatty | PWY-5367: petroselinate | 0.82 | 2.9e-03 | 0.95 | PWY- | petroselinate | Blautia | Ruminococcus_torques |
| acids | biosynthesis\|g__Blautia.s__Rumino- | | | | 5367 | biosynthesis | | |
| | coccus_torques | | | | | | | |
| miscel | PWY-5837: 1,4-dihydroxy-2- | 0.81 | 0.01 | 0.95 | PWY- | 1,4- | Lacto- | Lactococcus_lactis |
| laneous | naphthoate biosynthesis | | | | 5837 | dihydroxy-2- | coccus | |
| | I\|g__Lactococcus.s__Lacto- | | | | | naphthoate | | |
| | coccus_lactis | | | | | biosynthesis I | | |
| miscel- | PWY-5791: 1,4-dihydroxy-2- | 0.81 | 0.01 | 0.95 | PWY- | 1,4- | Lacto- | Lactococcus_lactis |
| laneous | naphthoate biosynthesis II | | | | 5791 | dihydroxy-2- | coccus | |
| | (plants)\|g__Lactococcus.s__Lacto- | | | | | naphthoate | | |
| | coccus_lactis | | | | | biosynthesis II | | |
| | | | | | | (plants) | | |
| miscel- | PWY-6163: chorismate | 0.45 | 7.1e-05 | 0.98 | PWY- | chorismate | Strepto- | Streptococcus_thermo- |
| laneous | biosynthesis from 3- | | | | 6163 | biosynthesis | coccus | philus |
| | dehydroquinate\|g__Strepto- | | | | | from 3- | | |
| | coccus.s__Streptococcus_thermophilus | | | | | dehydroquinate | | |
| miscel- | ARO-PWY: chorismate | 0.85 | 0.01 | 0.95 | ARO- | chorismate | Clostrid- | Clostridium_symbio- |
| laneous | biosynthesis | | | | PWY | biosynthesis I | ium | sum |
| | I\|g__Clostridium.s__Clostridium_symbio- | | | | | | | |
| | sum | | | | | | | |
| miscel- | ARO-PWY: chorismate | 0.41 | 1.3e-04 | 0.98 | ARO- | chorismate | Strepto- | Streptococcus_thermo- |
| laneous | biosynthesis | | | | PWY | biosynthesis I | coccus | philus |
| | I\|g__Streptococcus.s__Strepto- | | | | | | | |
| | coccus_thermophilus | | | | | | | |
| miscel- | COA-PWY-1: coenzyme A | 0.81 | 2.0e-03 | 0.96 | COA- | coenzyme A | Lacto- | Lacto- |
| laneous | biosynthesis II | | | | PWY-1 | biosynthesis II | bacillus | bacillus_casei_para- |
| | (mammalian)\|g__Lactobacillus.s__Lacto- | | | | | (mammalian) | | casei |
| | bacillus_casei_paracasei | | | | | | | |
| miscel- | COA-PWY-1: coenzyme A | 0.91 | 0.01 | 0.96 | COA- | coenzyme A | Strepto- | Streptococcus_para- |
| laneous | biosynthesis II | | | | PWY-1 | biosynthesis II | coccus | sanguinis |
| | (mammalian)\|g__Streptococcus.s__Strepto- | | | | | (mammalian) | | |
| | coccus_parasanguinis | | | | | | | |
| miscel- | PWY-3841: folate | 0.82 | 0.01 | 0.95 | PWY- | folate | Lacto- | Lactococcus_lactis |
| laneous | transformations | | | | 3841 | transformations | coccus | |
| | II\|g__Lactococcus.s__Lacto- | | | | | II | | |
| | coccus_lactis | | | | | | | |
| miscel- | PWY-3841: folate | 0.41 | 1.1e-04 | 0.96 | PWY- | folate | Strepto- | Streptococcus_thermo- |
| laneous | transformations | | | | 3841 | transformations | coccus | philus |
| | II\|g__Streptococcus.s__Strepto- | | | | | II | | |
| | coccus_thermophilus | | | | | | | |
| miscel- | PWY-5177: glutaryl-CoA | 0.81 | 0.01 | 0.95 | PWY- | glutaryl-CoA | Copro- | Coprococcus_catus |
| laneous | degradation\|g__Coprococcus.s__Copro- | | | | 5177 | degradation | coccus | |
| | coccus_catus | | | | | | | |
| miscel- | P461-PWY: hexitol | 0.81 | 3.5e-06 | 0.95 | P461- | hexitol | Lacto- | Lacto- |
| laneous | fermentation to lactate, formate, | | | | PWY | fermentation | bacillus | bacillus_casei_para- |
| | ethanol and | | | | | to lactate, | | casei |
| | acetate\|g__Lactobacillus.s__Lacto- | | | | | formate, | | |
| | bacillus_casei_paracasei | | | | | ethanol and | | |
| | | | | | | acetate | | |
| miscel- | PWY-7237: myo-, chiro- and | 0.85 | 0.01 | 0.95 | PWY- | myo-, chiro- | Clostrid- | Clostridium_symbio- |
| laneous | scillo-inositol | | | | 7237 | and scillo- | ium | sum |
| | degradation\|g__Clostridium.s__Clostrid- | | | | | inositol | | |
| | ium_symbiosum | | | | | degradation | | |
| miscel- | PWY-4242: pantothenate and | 0.83 | 0.01 | 0.95 | PWY- | pantothenate | Lacto- | Lactococcus_lactis |
| laneous | coenzyme A biosynthesis | | | | 4242 | and coenzyme | coccus | |
| | III\|g__Lactococcus.s__Lacto- | | | | | A biosynthesis | | |
| | coccus_lactis | | | | | III | | |
| miscel- | PEPTIDOGLYCANSYN-PWY: | 0.81 | 8.7e-04 | 0.95 | PEPTIDO- | peptidoglycan | Lacto- | Lactococcus_lactis |
| laneous | peptidoglycan biosynthesis I | | | | GLYCAN- | biosynthesis I | coccus | |
| | (meso-diaminopimelate | | | | SYNPWY | (meso- | | |
| | containing)\|g__Lactococcus.s__Lacto- | | | | | diamino- | | |
| | coccus_lactis | | | | | pimelate | | |
| | | | | | | containing) | | |
| miscel- | PWY-6385: peptidoglycan | 0.83 | 0.01 | 0.95 | PWY- | peptidoglycan | Blautia | Ruminococcus_torques |
| laneous | biosynthesis III | | | | 6385 | biosynthesis | | |
| | (mycobacteria)\|g__Blautia.s__Rumino- | | | | | III | | |
| | coccus_torques | | | | | (mycobacteria) | | |
| miscel- | PWY-5100: pyruvate | 0.7 | 1.1e-04 | 0.98 | PWY- | pyruvate | Strepto- | Streptococcus_thermo |
| laneous | fermentation to acetate and | | | | 5100 | fermentation | coccus | philus |
| | lactate | | | | | to acetate and | | |
| | II\|g__Streptococcus.s__Strepto- | | | | | lactate II | | |
| | coccus_thermophilus | | | | | | | |
| miscel- | PWY-7111: pyruvate | 0.82 | 0.01 | 0.95 | PWY- | pyruvate | Copro- | Coprococcus_catus |
| laneous | fermentation to isobutanol | | | | 7111 | fermentation | coccus | |
| | (engineered)\|g__Coprococcus.s__Copro- | | | | | to isobutanol | | |
| | coccus_catus | | | | | (engineered) | | |
| miscel- | PWY-7111: pyruvate | 0.85 | 4.6e-03 | 0.96 | PWY- | pyruvate | Lacto- | Lactococcus_lactis |
| laneous | fermentation to isobutanol | | | | 7111 | fermentation | coccus | |
| | (engineered)\|g__Lactococcus.s__Lacto- | | | | | to isobutanol | | |
| | coccus_lactis | | | | | (engineered) | | |
| miscel- | PWY-7111: pyruvate | 0.88 | 0.01 | 0.96 | PWY- | pyruvate | Strepto- | Streptococcus_para- |
| laneous | fermentation to isobutanol | | | | 7111 | fermentation | coccus | sanguinis |
| | (engineered)\|g__Streptococcus.s__Strepto- | | | | | to isobutanol | | |
| | coccus_parasanguinis | | | | | (engineered) | | |
| miscel- | PWY-7111: pyruvate | 0.41 | 2.8e-04 | 0.95 | PWY- | pyruvate | Strepto- | Streptococcus_thermo- |
| aneous | fermentation to isobutanol | | | | 7111 | fermentation | coccus | philus |
| | (engineered)\|g__Streptococcus.s__Strepto- | | | | | to isobutanol | | |
| | coccus_thermophilus | | | | | (engineered) | | |
| miscel- | PWY-6151: S-adenosyl-L- | 0.7 | 4.0e-03 | 0.95 | PWY- | S-adenosyl-L- | Anaero- | Anaerotruncus_coliho- |
| laneous | methionine cycle | | | | 6151 | methionine | truncus | minis |
| | I\|g__Anaerotruncus.s__Anaero- | | | | | cycle I | | |
| | truncus_colihominis | | | | | | | |
| miscel- | PWY-6151: S-adenosyl-L- | 0.81 | 1.9e-04 | 0.96 | PWY- | S-adenosyl-L- | Lacto- | Lacto- |
| laneous | methionine cycle | | | | 6151 | methionine | coccus | bacillus_casei_para- |
| | I\|g__Lactobacillus.s__Lacto- | | | | | cycle I | | casei |
| | bacillus_casei_paracasei | | | | | | | |
| miscel- | PWY-6151: S-adenosyl-L- | 0.81 | 0.01 | 0.95 | PWY- | S-adenosyl-L- | Lacto- | Lactococcus_lactis |
| laneous | methionine cycle | | | | 6151 | methionine | coccus | |
| | I\|g__Lactococcus.s__Lacto- | | | | | cycle I | | |
| | coccus_lactis | | | | | | | |
| miscel- | PWY-6151: S-adenosyl-L- | 0.82 | 0.01 | 0.95 | PWY- | S-adenosyl-L- | Blautia | Ruminococcus_torques |
| laneous | methionine cycle | | | | 6151 | methionine | | |
| | I\|g__Blautia.s__Rumino- | | | | | cycle I | | |
| | coccus_torques | | | | | | | |
| miscel- | PWY-6151: S-adenosyl-L- | 0.41 | 4.3e-04 | 0.98 | PWY- | S-adenosyl-L- | Strepto- | Streptococcus_thermo- |
| laneous | methionine cycle | | | | 6151 | methionine | coccus | philus |
| | I\|g__Streptococcus.s__Strepto- | | | | | cycle I | | |
| | coccus_thermophilus | | | | | | | |
| miscel- | P125-PWY: superpathway of | 0.85 | 0.01 | 0.98 | P125- | superpathway | Lacto- | Lactococcus_lactis |
| laneous | (R,R)-butanediol | | | | PWY | of (R,R)- | coccus | |
| | biosynthesis\|g__Lactococcus.s__Lacto- | | | | | butanediol | | |
| | coccus_lactis | | | | | biosynthesis | | |
| miscel- | MET-SAM-PWY: superpathway | 0.81 | 2.0e-03 | 0.95 | MET- | superpathway | Lacto- | Lactococcus_lactis |
| laneous | of S-adenosyl-L-methionine | | | | SAM- | of 5-adenosyl- | coccus | |
| | biosynthesis\|g__Lactococcus.s__Lacto- | | | | PWY | L-methionine | | |
| | coccus_lactis | | | | | biosynthesis | | |
| miscel- | THISYNARA-PWY: | 0.83 | 0.01 | 0.95 | THISYNARA- | superpathway | Blautia | Ruminococcus_torques |
| laneous | superpathway of thiamin | | | | PWY | of thiamin | | |
| | diphosphate biosynthesis III | | | | | diphosphate | | |
| | (eukaryotes)\|g__Blautia.s__Rumino- | | | | | biosynthesis | | |
| | coccus_torques | | | | | III | | |
| | | | | | | (eukaryotes) | | |
| miscel- | PWY-7357: thiamin formation | 0.87 | 2.9e-03 | 0.95 | PWY- | thiamin | Lacto- | Lactococcus_lactis |
| laneous | from pyrithiamine and | | | | 7357 | formation | coccus | |
| | oxythiamine | | | | | from | | |
| | (yeast)\|g__Lactococcus.s__Lacto- | | | | | pyrithiamine | | |
| | coccus_lactis | | | | | and | | |
| | | | | | | oxythiamine | | |
| | | | | | | (yeast) | | |
| miscel- | UDPNAGSYN-PWY: UDP-N- | 0.85 | 4.2e-03 | 0.95 | UDPNAG- | UDP-N- | Lacto- | Lactococcus_lactis |
| laneous | acetyl-D-glucosamine | | | | SYNPWY | acetyl-D- | coccus | |
| | biosynthesis | | | | | glucosamine | | |
| | I\|g__Lactococcus.s__Lacto- | | | | | biosynthesis I | | |
| | coccus_lactis | | | | | | | |
| miscel- | UDPNAGSYN-PWY: UDP-N- | 0.41 | 7.1e-05 | 0.95 | UDPNAG- | UDP-N- | Strepto- | Streptococcus_thermo- |
| laneous | acetyl-D-glucosamine | | | | SYNPWY | acetyl-D- | coccus | philus |
| | biosynthesis | | | | | glucosamine | | |
| | I\|g__Streptococcus.s__Strepto- | | | | | biosynthesis I | | |
| | coccus_thermophilus | | | | | | | |
| miscel- | PWY-6387: UDP-N- | 0.82 | 8.7e-04 | 0.95 | PWY- | UDP-N- | Lacto- | Lactococcus_lactis |
| laneous | acetylmuramoyl-pentapeptide | | | | 6387 | acetylmuramoyl- | coccus | |
| | biosynthesis I (meso- | | | | | pentapeptide | | |
| | diaminopimelate | | | | | biosynthesis I | | |
| | containing)\|g__Lactococcus.s__Lacto- | | | | | (meso- | | |
| | coccus_lactis | | | | | diamino- | | |
| | | | | | | pimelate | | |
| | | | | | | containing) | | |
| miscel- | PWY-6386: UDP-N- | 0.82 | 9.6e-04 | 0.95 | PWY- | UDP-N-acetyl- | Lacto- | Lactococcus_lactis |
| laneous | acetylmuramoyl-pentapeptide | | | | 6386 | muramoyl- | coccus | |
| | biosynthesis II (lysine- | | | | | pentapeptide | | |
| | containing)\|g__Lactococcus.s__Lacto- | | | | | biosynthesis II | | |
| | coccus_lactis | | | | | (lysine- | | |
| | | | | | | containing) | | |
| miscel- | PWY-6386: UDP-N- | 0.41 | 3.7e-04 | 0.95 | PWY- | UDP-N- | Strepto- | Streptococcus_thermo- |
| laneous | acetylmuramoyl-pentapeptide | | | | 6386 | acetylmuramoyl- | coccus | philus |
| | biosynthesis II (lysine- | | | | | pentapeptide | | |
| | containing)\|g__Streptococcus.s__Strepto- | | | | | biosynthesis II | | |
| | coccus_thermophilus | | | | | (lysine- | | |
| | | | | | | containing) | | |

### Physiologically-Relevant Levels of Endogenous Intestinal Bile Acids Lead to Significant Inhibition of Bacterial Growth Under Both Aerobic and Anaerobic Conditions.

Gram-Positive Bacteria are Generally More Sensitive to Growth Inhibition by Bile Acids than Gram-Negative Bacteri Anaerostipes caccae

in combination

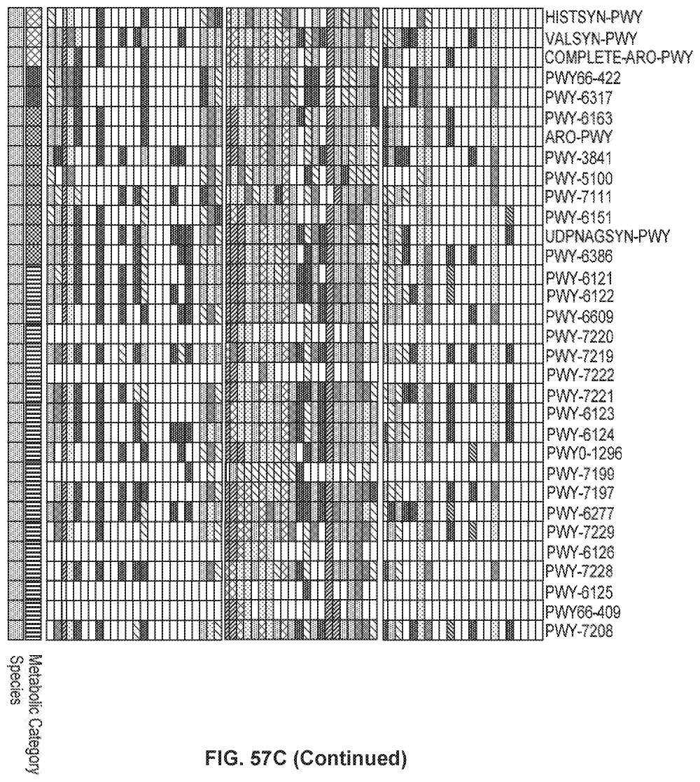

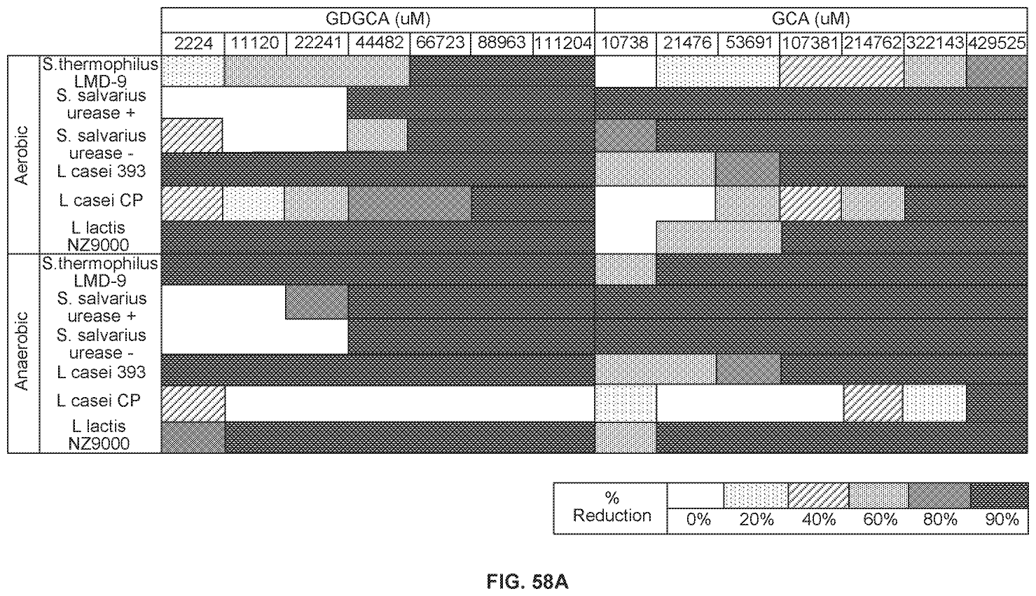

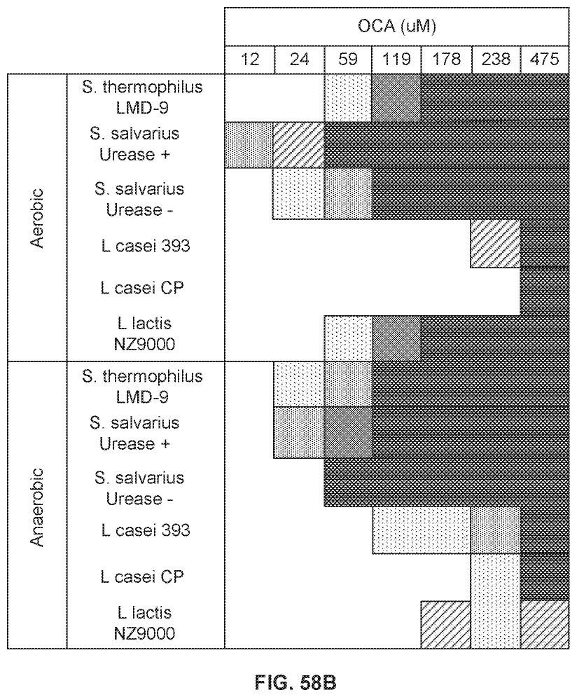

a (Begley et al., FEMS Microbiol Rev. 29, 625-651 (2005)), and conducted genomic analysis is consistent with the notion that specific Gram-positive taxa become proportionally more abundant during OCA administration due to enhanced proliferation. FXR-dependent inhibition of endogenous bile acid synthesis by OCA may reduce the growth inhibitory effects on Gram-positive bacterial species that are normally sensitive to bile acids. To determine if the three bacterial species with the greatest representation of pathways associated with OCA (FIG. 57A) are sensitive to bile acids, the species-specific minimal inhibitory concentrations (MIC) of the two most predominant bile acids in the human small intestine, glycochenodeoxycholic acid (GCDCA) and glycocholic acid (GCA) were determined (FIG. 58A). Since the environment of the small intestinal lumen may transition from an aerobic to anaerobic state along its length (He et al., Proceedings of the National Academy of Sciences of the United States of America, 96, 4586-4591 (1999)), MICs were determined under both aerobic and anaerobic conditions. Although each species showed variable growth inhibition to the two bile acids under both aerobic and anaerobic conditions, the growth of all three species was significantly inhibited at the physiologically relevant concentrations (millimolar range) of bile acids normally found in the small intestine (Northfield and McColl, Gut, 14, 513-518 (1973)). Bile acid deconjugating enzymes have been shown to enhance bile acid tolerance (Begley et al., FEMS Microbiol Rev. 29, 625-651 (2005)). Consistent with this notion, it has been shown that bile salt hydrolases (BSH), enzymes that are unique to bacteria resident in the mammalian gut where they can enhance bile acid tolerance (Begley et al., FEMS Microbiol Rev. 29, 625-651 (2005)), are missing from the genome of S. thermophilus, which is induced by OCA. It can be supported by showing that these species are sensitive to growth inhibition in vitro when exposed to physiologically-relevant concentrations of conjugated bile acids found in the small intestine. By contrast, growth inhibition was not observed upon exposure to OCA, suggesting that FXR-dependent inhibition of endogenous bile acid secretion is responsible for the observed change in the gut microbiota and is not a direct effect of OCA. As shown in FIG. 58A, bile acid deconjugating enzymes were absent from all available reference genomes of S. thermophilus, a finding consistent with the bile acid sensitivity observed in vitro. Since OCA is a bile acid analogue possibly capable of inhibiting bacterial growth, the MICs of OCA for the same bacterial species were determined (FIG. 58B). Although OCA was also able to inhibit growth of all three bacterial species, there was minimal to no inhibition of growth at OCA concentrations calculated to be reached in the human small intestine (˜40 mM at a 10 mg/day dose) (Zhang et al., Pharmacol Res Perspect, 5 (2017)). These findings support the notion that OCA treatment leads to the increased growth of bile acid sensitive bacterial taxa by suppressing endogenous bile acid synthesis.

### OCA Treatment in Mice Inhibits Endogenous Luminal Bile Acid Levels and Leads to Increased Gram-Positive Bacteria, Specifically in the Small Intestine.

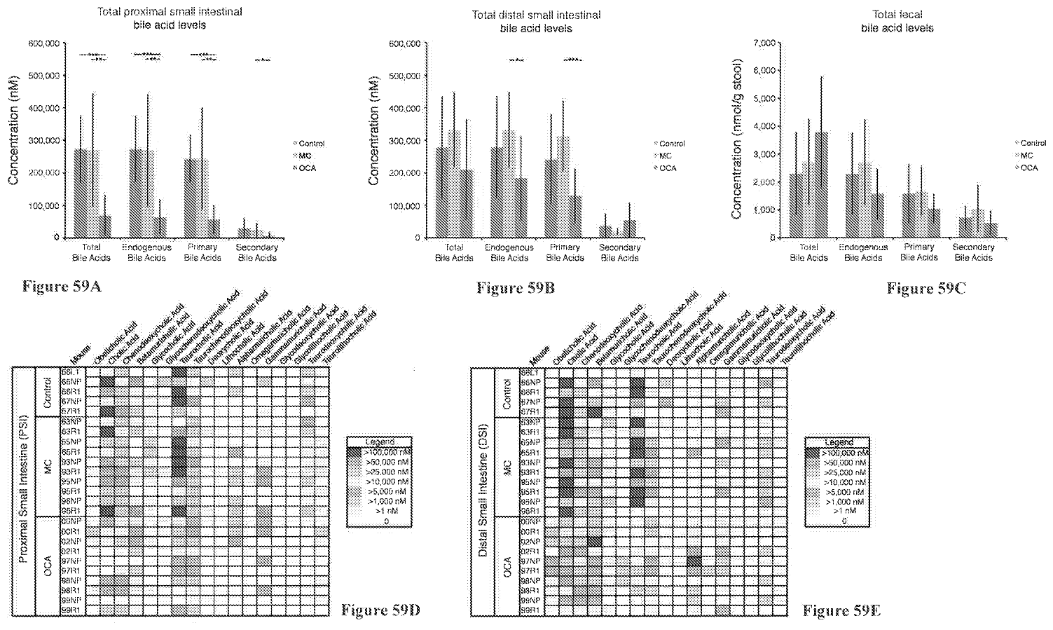

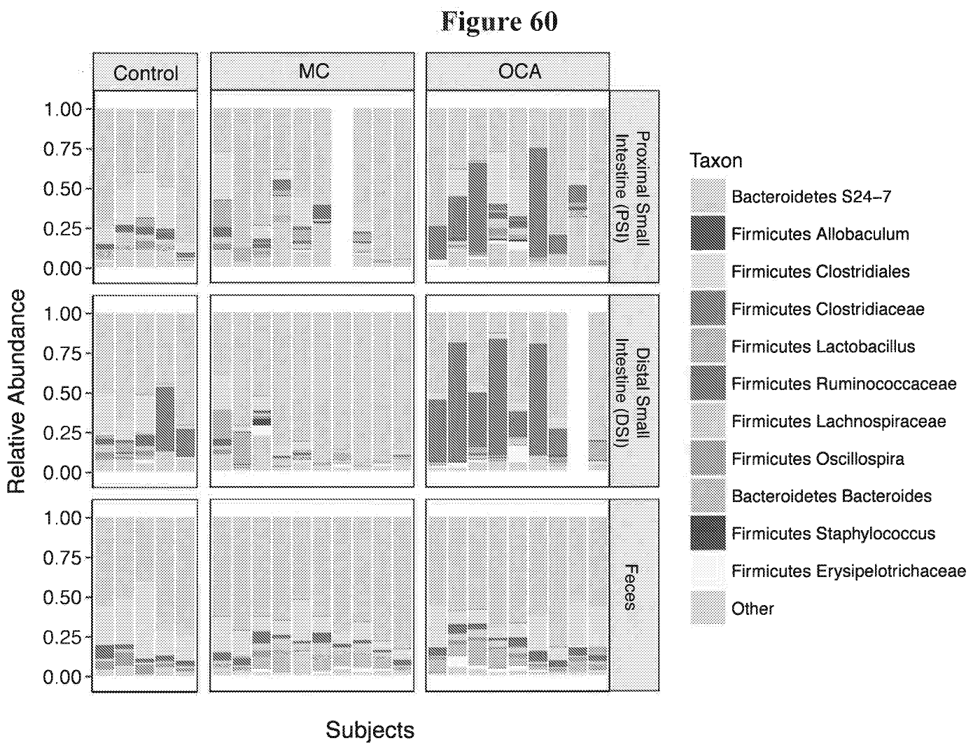

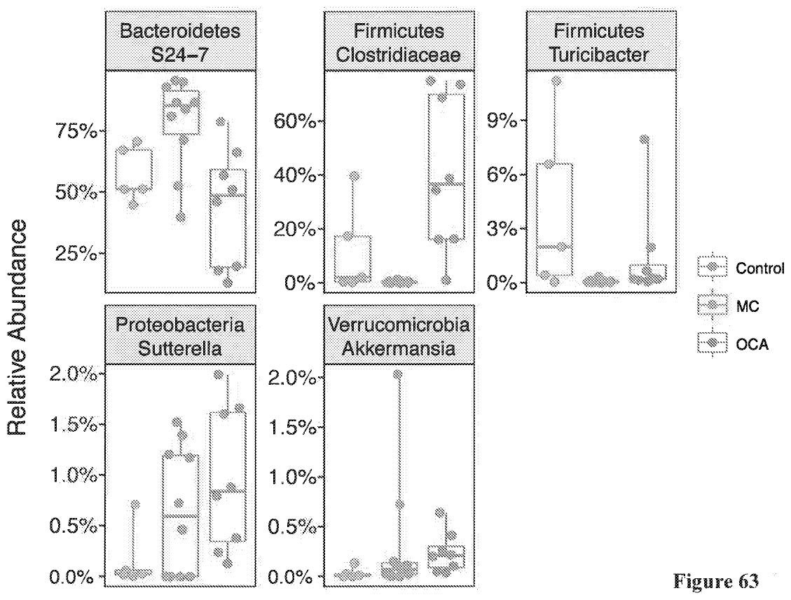

The bacterial species that increase most prominently upon OCA treatment have been reported to represent a significant proportion of the small intestinal microbiota, but are very minor constituents in stool. For example, Streptococci represent as much as 20% of the human small intestinal microbiota by 16S tagged sequencing (Dlugosz et al., Sci Rep-Uk, 5 (2015)); El Aidy et al., Curr Opin Biotechnol., 32, 14-20 (2015)). Some of these bacteria are environmental organisms used in the manufacturing of food introduced into the small intestine with diet, including Streptococcus thermophilus, Bifidobacterium breve, Lactobacillus casei, and Lactococcus lactis (Brigidi et al., International Journal of Food Microbiology, 81, 203-209 (2003); Derrien and van Hylckama Vlieg, Trends Microbiol., 23, 354-366 (2015); Stiles and Holzapfel, International Journal of Food Microbiology, 36, 1-29 (1997)). To determine whether OCA treatment can alter the gut microbiota composition specifically in the small intestine, mice were treated with OCA for 14 days, the microbiota composition in the proximal and distal small intestine, as well as the stool, and quantified bile acids were characterized. Since OCA was prepared in methylcellulose, an additional methylcellulose control group was included due to its previously described effect on fecal bile acid levels (Cox et al., FASEB J 27, 692-702 (2013)). Quantification of luminal bile acid concentrations revealed a significant reduction of endogenous primary bile acids that was greatest in the proximal small intestine but was also observed in the distal small intestine, with no effect in the feces (FIGS. 59A-59C). The largest consistent decreases were observed in the primary bile acids cholic and taurocholic acids, with smaller and less consistent decreases in several of the less abundant bile acids (FIGS. 59D and 59E). OCA was detectable in the small intestinal lumen of treated mice, where it contributed minimally to the total concentration of bile acids (FIGS. 59A-59E). Also notable was the quantitative difference in the absolute levels of luminal bile acids in the small intestine (approximately 100-fold greater than in the feces, FIGS. 59A-59C); this is consistent with enterohepatic reabsorption of over 95% of bile acids in the terminal ileum of the small intestine (de Aguiar Vallim et al., Cell Metabolism, 17, 657-669 (2013)). The overall composition of the small intestinal microbiota in the OCA group, analyzed by 16S tagged sequencing, was different from both control groups. This was due to the increased abundance in the Clostriciaceae family of Firmicutes in both the proximal and distal small intestine of OCA-treated mice, but not in the feces (FIGS. 60 and 63 (microbiota composition in the proximal small intestine (PSI), distal small intestine (DSI), and feces of mice following 14 days of gavage with either water (control), 0.5% methylcellulose (MC), or 0.5% methylcellulose with 10 mg/kg obeticholic acid (OCA)). Collectively, these results demonstrate that treatment with OCA in mice leads to a significant reduction in luminal bile acid concentrations specifically in the small intestine, where there is a concurrent increase in Gram-positive bacteria. Neither of these alterations was observed in fecal samples, showing that the activation of FXR by OCA and the resulting inhibition of endogenous bile acid synthesis impacts the small intestinal microbiota. These results, combined with the effects of OCA treatment on the abundance of bacterial taxa known to predominate in the small intestine, provide compelling evidence for the role of FXR in the dynamics of the human small intestinal microbiota.

### A Robust Taxonomic Signature for FXR Activation in the Human Gut Microbiome.

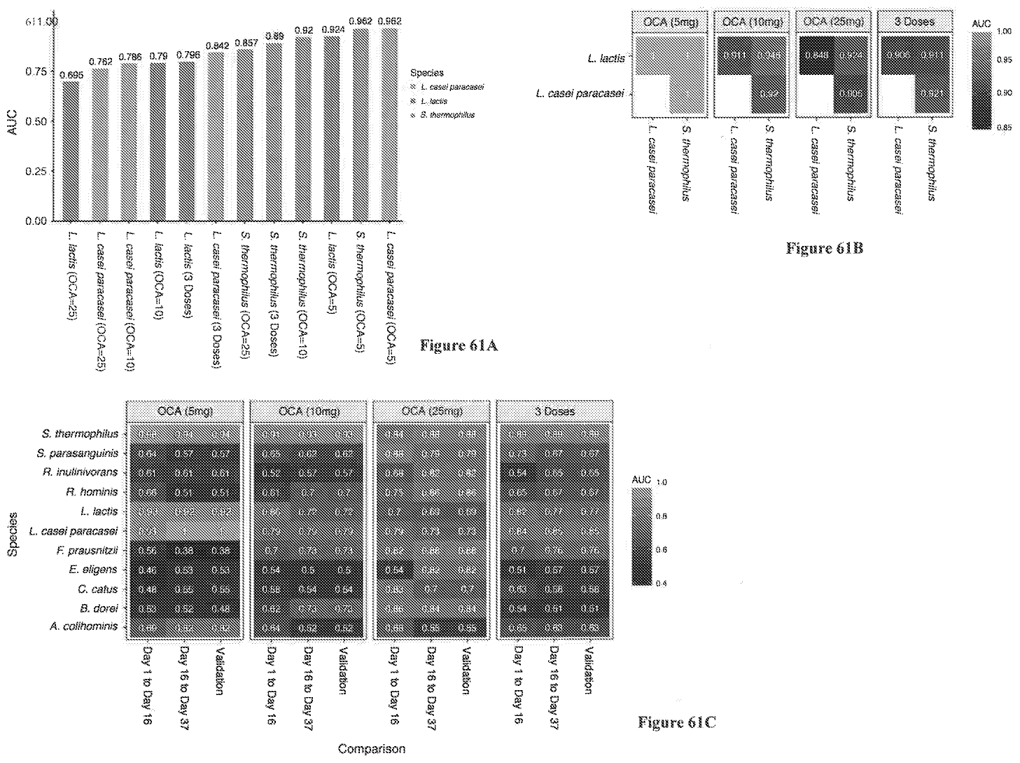

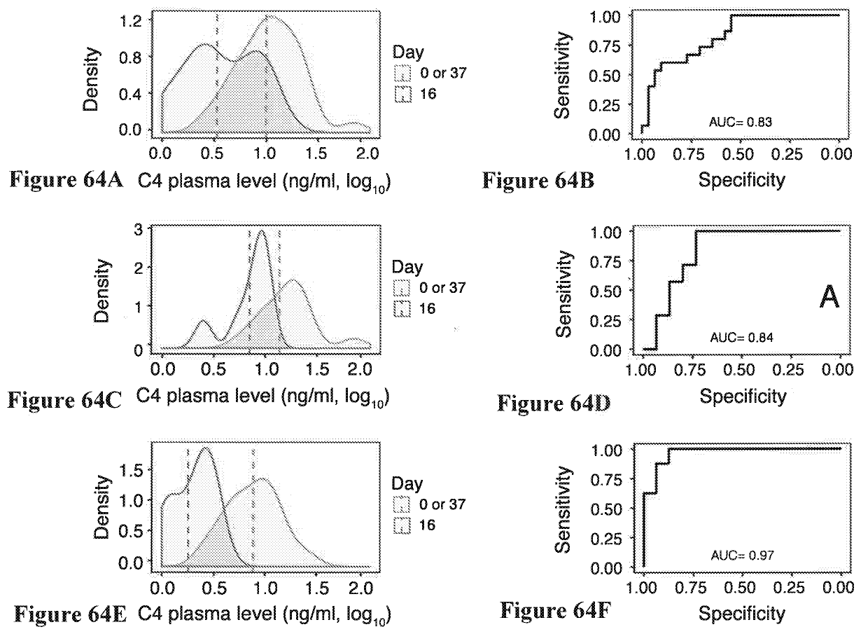

Growing evidence suggests that the composition of the gut microbiome might have value as a biomarker for drug metabolism (Klaassen and Cui, Drug Metabolism and Disposition: the Biological Fate of Chemicals, 43, 1505-1521 (2015)) and diet (Zeevi et al., Cell, 163, 1079-1094 (2015)) for personalized medicine, and discriminatory indices have been developed to categorize specific disease processes involving infections (Buffie et al., Nature, 517, 205-208 (2015)), liver disease (Loomba et al., Cell Metabolism, 25, 1054-1062 e1055 (2017); Qin et al., Nature, 513, 59-64 (2014)), and inflammatory bowel disease (Barber et al., Am. J. Gastroenterol., 111, 1816-1822 (2016)). The robust and reversible response of bacterial taxa to OCA treatment suggests that specific bacterial species, alone or in combination, might predict FXR-dependent inhibition of bile acid synthesis. ROC curves were generated and the area under the ROC curve (AUC) from logistic regression was calculated for each species characterized in our shotgun metagenomic dataset. The taxa that most accurately predicted treatment with the three doses of OCA, individually or combined, were L. casei-paracasei, L. lactis, and S. thermophilus (FIG. 61A). Species with high AUC values (AUC>0.8) from at least one model are shown. The pseudo-validation AUCs were obtained by applying the logistic model derived from the Day 1 vs. Day 16 dataset (i.e., training set) to the Day 16 vs. Day 37 dataset (i.e., validation set). For each of these species, the highest AUCs were observed at the OCA 5 mg dose. A combination of any two of these three species results in an AUC close to “1” for the 5 mg OCA dose (FIG. 61B). This taxonomic signature exceeds the performance of C4 as a predictor of OCA administration at the 5 mg dose (FIG. 64). ROC analysis was performed for these three species, as well as a number of additional taxa, to determine the AUC at 5, 10, 25 mg doses, both independently and combined. To provide an assessment of the predictability of the models, data from Day 0 to 17 were used a training set, and data from Day 17 to 37 as a pseudovalidation set (FIG. 61C). In general, the AUCs between these two intervals were very similar, which demonstrates the robust and reproducible nature of these associations. The data was measured over three time points from the same set of individuals were used in the ROC analyses. Although the three species described in FIG. 61A all had the highest AUCs at the 5 mg dose of OCA, two species showed a dose-dependent increase in AUC. Faecalibacterium prausnitzii and Bacteroides dorei both had AUCs of approximately 0.5 at the 5 mg dose, 0.7 at the 10 mg dose, and 0.85 at the 25 mg dose. Ultimately, taxonomic features that are maximally sensitive to the lowest dose of OCA, combined with others that show a more dynamic dosedependent association, may prove useful in predicting clinical responses induced by OCA and likely other FXR agonists.

Examples 1-7 are provided to illustrate certain embodiments of the present disclosure (Gary D. Wu, Modulation of Gut Microbiota by the Bile Acid Derivative Obeticholic Acid, abstract and presentation at Keystone Symposia, Mar. 3-7, 2017, Monterey, Calif., USA; FXR-Dependent Modification Of The Human Small Intestinal Microbiome (2922417), has been selected for a lecture presentation during Digestive Disease Week® (DDW) at the Walter E. Washington Convention Center in Washington, D.C., Jun. 2-5, 2018; Friedman et al., FXR-Dependent Modulation of the Human Small Intestinal Microbiome by the Bile Acid Derivative Obeticholic Acid.; manuscript is being submitted for publication).

### EQUIVALENTS

Those skilled in the art will recognize, or be able to ascertain using no more than routine experimentation, many equivalents to the specific embodiments and methods described herein. Such equivalents are intended to be encompassed by the scope of the present application. All patents, patent applications, and literature references cited herein are hereby expressly incorporated by reference.

1. A pharmaceutical composition comprising a compound of formula I: 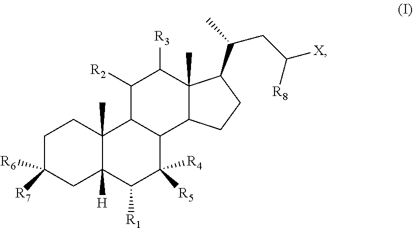
or a pharmaceutically acceptable salt or amino acid conjugate thereof, wherein:
R₁ is unsubstituted C₁-C₆ alkyl;
R₂ is H or hydroxyl;
R₃ is H or hydroxyl;
R₄, R₅, R₆, and R₇ are each independently H or hydroxyl;
R₈ is H or unsubstituted C₁-C₆ alkyl;
X is C(O)OH, C(O)NH(CH₂)ₘSO₃H, C(O)NH(CH₂)ₙCO₂H, or OSO₃H;
m is 1, 2, or 3; and
n is 1, 2, or 3,
and one or more gut microbiome species, and a pharmaceutically acceptable carrier.

2-17. (canceled)

18. The pharmaceutical composition of claim 1, wherein the compound is of formula Ib-1 or Ib-2 or Ic: 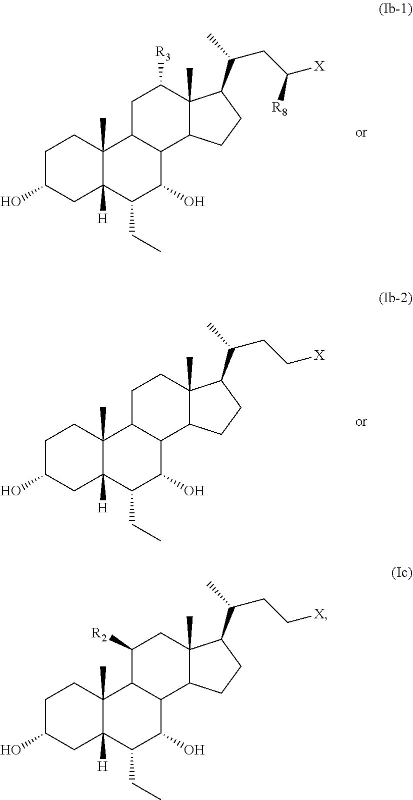
or a pharmaceutically acceptable salt or amino acid conjugate thereof.

19. (canceled)

20. The pharmaceutical composition of claim 1, wherein the compound is: 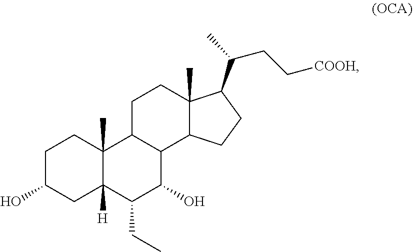 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]
or a pharmaceutically acceptable salt or amino acid conjugate thereof.

21. The pharmaceutical composition of claim 1, wherein the compound is: 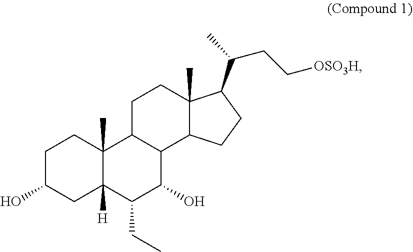
or a pharmaceutically acceptable salt or amino acid conjugate thereof.

22. The pharmaceutical composition of claim 1, wherein the compound is: 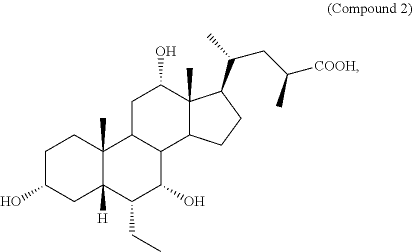 [SMILES: CC[C@H]1[C@@H](O)C2C3CC[C@H]([C@H](C)C[C@H](C)C(=O)O)[C@@]3(C)[C@@H](O)CC2[C@@]2(C)CC[C@@H](O)C[C@@H]12]
or a pharmaceutically acceptable salt or amino acid conjugate thereof.

23. The pharmaceutical composition of claim 1, wherein the compound is: 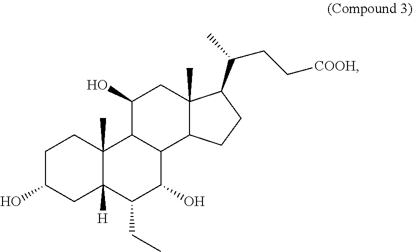 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]
or a pharmaceutically acceptable salt or amino acid conjugate thereof.

24. The pharmaceutical composition of claim 1, wherein the one or more gut microbiome species is a member in a family selected from: Actinomycetaceae, Bogoriellaceae, Brevibacteriaceae, Cellulomonadaceae, Acholeplasmataceae, Acidithiobacillaceae, Alcanivoracaceae, Alteromonadaceae, Blattabacteriaceae, Cardiobacteriaceae, Chlamydiaceae, Chromatiaceae, Clostridiales Family XIII. Incertae Sedis, Cyclobacteriaceae, Dehalococcoidaceae, Desulfobacteraceae, Desulfobulbaceae, Ectothiorhodospiraceae, Elusimicrobiaceae, Entomoplasmataceae, Erythrobacteraceae, Gallionellaceae, Halanaerobiaceae, Jonesiaceae, Kofleriaceae, Leptospiraceae, Methanobacteriaceae, Methylococcaceae, Methylophilaceae, Myxococcaceae, Nitrosomonadaceae, Nitrospiraceae, Oceanospirillaceae, Oscillospiraceae, Piscirickettsiaceae, Propionibacteriaceae, Pseudoalteromonadaceae, Puniceicoccaceae, Rickettsiaceae, Rubrobacteraceae, Shewanellaceae, Spirochaetaceae, Spiroplasmataceae, Sutterellaceae, Syntrophomonadaceae, Thermaceae, Corynebacteriaceae, Dermabacteraceae, Dietziaceae, Geodermatophilaceae, Gordoniaceae, Intrasporangiaceae, Microbacteriaceae, Micrococcaceae, Micromonosporaceae, Mycobacteriaceae, Nocardiaceae, Promicromonosporaceae, Propionibacterineae, Streptomycetaceae, Micrococcineae, Bifidobacteriaceae, Coriobacteriaceae, Deinococcaceae, Halobacteroidaceae, Alicyclobacillaceae, Bacillaceae, Bacillales Incertae Sedis XI, Listeriaceae, Paenibacillaceae, Planococcaceae, Staphylococcaceae, Aerococcaceae, Carnobacteriaceae, Enterococcaceae, Lactobacillaceae, Leuconostocaceae, Streptococcaceae, Christensenellaceae, Clostridiaceae, Ruminococcaceae, Family XIII Incertae Sedis, Peptostreptococcaceae, Family XI Incertae Sedis, Lachnospiraceae, Eubacteriaceae, Erysipelotrichaceae, Erysipelotrichaceae XVI, Erysipelotrichaceae XVII, Erysipelotrichaceae XVIII, Acidiaminococcaceae, Peptococcaceae, Veillonellaceae, Bacteroidaceae, Porphyromonadaceae, Prevotellaceae, Rikenellaceae, Cytophagaceae, Flavobacteriaceae, Chitinophagaceae, Sphingobacteriaceae, Fusobacteriaceae, Leptotrichiaceae, Victivallaceae, Planctomycetaceae, Caulobacteraceae, Aurantimonadaceae, Bradyrhizobiaceae, Brucellaceae, Hyphomicrobiaceae, Methylobacteriaceae, Phyllobacteriaceae, Rhizobiaceae, Xanthobacteraceae, Rhodobacteraceae, Acetobacteraceae, Rhodospirillaceae, Sphingomonadaceae, Alcaligenaceae, Burkholderiaceae, Comamonadaceae, Oxalobacteraceae, Suterellaceae, Neisseriaceae, Rhodocyclaceae, Desulfovibrionaceae, Campylobacteraceae, Helicobacteraceae, Aeromonadaceae, Succinivibrionaceae, Enterobacteriaceae, Pasteurellaceae, Moraxellaceae, Pseudomonadaceae, Vibrionaceae, Sinobacteraceae, Xanthomonadaceae, Brachyspiraceae, Synergistaceae, Mycoplasmataceae, and Verrucomicrobiaceae.

25. The pharmaceutical composition of claim 24, wherein the one or more gut microbiome species is gram positive.

26. The pharmaceutical composition of claim 25, wherein the one or more gut microbiome species is selected from Bifidobacterium breve, Bifidobacterium longum, Lactobacillus casei, Lactobacillus paracasei, Pediococcus pentosaceus, Lactococcus lactis, Streptococcus parasanguinis, Streptococcus salivarius, Streptococcus thermophilus, Ruminococcus bromii, Ruminococcus torques, Anaerotruncus unclassified, Subdoligranulum unclassified, Clostridium difficile, Blautia (Ruminococcus) obeum, Dorea longicatena, Eubacterium ramulus, Ruminococcus gnavus, Ruminococcus torques, Lachnospiracea bacterium 5_1_63FAA, Lachnospiraceae bacterium 3_1_57FAA_CT1, and Lachnospiraceae bacterium 8_1_57FAA, Coprobacillus unclassified, Clostridium spiroforme, Clostridium symbiosum, Veillonella parvula, and Veillonella unclassified.

27. The pharmaceutical composition of claim 24, wherein the one or more gut microbiome species is gram negative.

28. The pharmaceutical composition of claim 27, wherein the one or more gut microbiome species is selected from Bacteroides ovatus, Bacteroides plebeius, Bacteroides uniformis, Bacteroidales ph8, Odoribacter splanchnicus, Paraprevotella clara, Paraprevotella unclassified, Alistipes putredinis, Alistipes shahii, Escherichia coli, and Akkermansia muciniphila.

29. The pharmaceutical composition of claim 1, wherein the one or more gut microbiome species is sensitive to growth inhibition by an endogenous bile acid.

30. The pharmaceutical composition of claim 1, wherein the one or more gut microbiome species is a human gut microbiome species.

31. The pharmaceutical composition of claim 1, wherein the compound or a pharmaceutically acceptable amino acid conjugate or salt thereof is present in the amount of 5-25 mg in the pharmaceutical composition.

32. The pharmaceutical composition of claim 1, wherein the pharmaceutical composition comprises the one or more gut microbiome species in the amount of 100-10¹² colony forming unit.

33. The pharmaceutical composition of claim 1, wherein the compound or a pharmaceutically acceptable amino acid conjugate or salt thereof is formulated for oral, parenteral, or topical administration.

34. The pharmaceutical composition of claim 33, wherein the compound or a pharmaceutically acceptable amino acid conjugate or salt thereof is formulated for oral administration.

35. The pharmaceutical composition of claim 1, wherein the one or more gut microbiome species is formulated for oral administration.

36. A method of treating or preventing an FXR mediated disease or condition or a disease or condition in which an abnormal composition of the gut microbiome is involved, comprising administering to a subject in need thereof a pharmaceutical composition of claim 1.

37-39. (canceled)

40. A method of enhancing the efficacy of an FXR ligand in treating or preventing a disease or condition, comprising administering to a subject in need thereof one or more gut microbiome species.

41-43. (canceled)
